(19) United States
(12) Patent Application Publication
(10) Pub. No.: US 2026/0060961 A1
(43) Pub. Date: Mar. 5, 2026

(54) SUBSTITUTED HETEROCYCLES AS RAS INHIBITORS

(71) Applicant: Jazz Pharmaceuticals Ireland Limited, Dublin (IE)

(72) Inventors: Clifford D. JONES, Macclesfield (GB); Andrew BELFIELD, Macclesfield (GB); Cinzia BORDONI, Cheshire (GB)

(21) Appl. No.: 19/106,853

(22) PCT Filed: Aug. 30, 2023
(86) PCT No.: PCT/EP2023/073850

§ 371 Date: Feb. 26, 2025

(30) Foreign Application Priority Data
Aug. 31, 2022 (GB) 2212641.1

(51) Int. Cl. A61K 31/4178; A61K 31/423; A61K 31/427; A61K 31/4439; A61K 31/5377; C07D 401/14; C07D 403/04; C07D 403/14; C07D 409/14; C07D 413/14; C07D 417/14

(52) U.S. Cl. CPC A61K 31/4178; A61K 31/423; A61K 31/427; A61K 31/4439; A61K 31/5377; C07D 401/14; C07D 403/04; C07D 403/14; C07D 409/14; C07D 413/14; C07D 417/14

(57) ABSTRACT

Provided herein are compounds identified as inhibitors of KRAS protein activity that can be used to treat various diseases and disorders, such as cancer.

### CROSS REFERENCE TO RELATED APPLICATIONS

This application claims the benefit of and priority to GB Application No. 2212641.1, filed Aug. 31, 2022, which is herein incorporated by reference in its entirety.

### FIELD OF INVENTION

The present disclosure relates to compounds and their methods of use. In particular, the disclosed compounds may be useful for inhibiting RAS proteins. More specifically, this disclosure relates to compounds for inhibiting KRAS^{G12C} proteins. The compounds of the disclosure may therefore be used in treating conditions mediated by RAS proteins. For example, the compounds may be used in treating cancer.

### BACKGROUND OF THE DISCLOSURE

RAS is the most commonly mutated oncogene in cancer (˜30%), with KRAS the most commonly mutated isoform accounting for ˜85% of RAS mutations. RAS proteins are a group of closely related monomeric globular proteins comprising 189 amino acids. These proteins are associated with the plasma membrane.

RAS proteins are small GTPases, known as G-proteins, that bind guanine nucleotides and hydrolyse GTP to GDP. They function as molecular switches, being ‘on’ when bound to GTP and ‘off’ when bound to GDP. When bound to GTP, the RAS protein can interact with other proteins. In order for the RAS proteins to be switched ‘off’ i.e. to hydrolyze GPT back to GDP, extrinsic proteins are required. These extrinsic proteins are called GTPase-activating proteins (GAPs) and increase the rate of conversion of GTP to GDP. In response to signals from receptor tyrosine kinases, guanine nucleotide exchange factors (GEFs) such as SOS1 and SOS2 facilitate nucleotide exchange converting RAS from GDP- to GTP-bound state. When GTP-bound, the intrinsic GTPase activity of RAS is stimulated by (GAPs) such as NF1, catalysing the conversion of RAS from GTP- to GDP-bound state. Thus, the intrinsic GTPase activity allows RAS to auto-inactivate downstream signalling. Mutations in RAS that reduce its intrinsic GTPase activity result in an accumulation of GTP-bound RAS leading to upregulation of effector pathways, causing increased cell proliferation. The most common oncogenic KRAS mutations are at amino acid position G12, G13 and Q61, with KRAS G12C being the most common activating mutation in lung cancer. KRAS mutations are also found in multiple other cancers including cervical cancer, multiple myeloma, stomach cancer, bladder cancer and uterine cancer. The role of KRAS^{G12C} in multiple tumour types makes it a particularly attractive target for developing small molecule inhibitors against.

For these reasons, there have been a number of recent patent applications concerned with compounds which are capable of modulating G12C mutant KRAS. For example, see WO 2018/218069, WO 2018/218070, WO 2018/218071, WO 2017/100546, WO 2018/064510, WO 2016/168540, WO 2017/015562, WO 2016/164675, WO 2015/054572, WO 2019/099524, WO 2017/201161, WO 2018/119183, WO 2019/051291, WO 2018/217651, WO 2019/110751, WO 2018/206539, US20190248767, and WO 2018/143315.

Still, there is a need in the art to provide alternative or improved compounds for inhibiting RAS proteins, specifically alternative or improved compounds for inhibiting KRAS proteins.

### SUMMARY OF THE DISCLOSURE

Accordingly, in various aspects, the present disclosure provides a compound of Formula (I-1) or Formula (I-2):

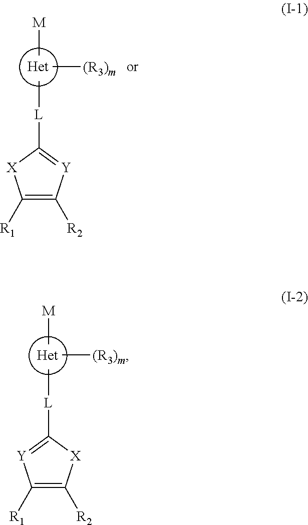

or a pharmaceutically acceptable salt, stereoisomer, or atropisomer thereof,
wherein:
X is N—R₄, O, or S;
Y is N or CH;

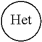

is a nitrogen-containing heterocyclyl;
L is a bond, alkylene, alkenylene, alkynylene, —C(O)—, or —S(O)₂—;
R₁ and R₂ are each independently aryl or heteroaryl;
R₃ is hydrogen, halogen, alkyl, hydroxy, alkoxy, —CN, —C(O)OR₅, or —S(O)₂NR₅R₆; or two R₃ groups attached to the same carbon atom form an oxo, cycloalkyl, or heterocyclyl; or two R₃ groups taken together with the carbon atoms to which they are attached form a cycloalkyl or heterocyclyl;
R₄ is alkyl, cycloalkyl, alkylenecycloalkyl, heterocyclyl, or alkyleneheterocyclyl;
R₅ and R₆ are each independently alkyl, cycloalkyl, heterocyclyl, aryl, heteroaryl, alkylenecycloalkyl, alkyleneheterocyclyl, alkylenearyl, or alkyleneheteroaryl;
M is

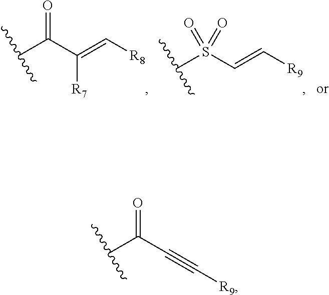

wherein
   R₇ is hydrogen, halogen, C₁-C₃alkyl, —C(O)—C₁-C₃alkyl, or —CN;
   R₈ is H, alkyl, —CF₃, —CHF₂, —CH₂F, —CH₂—Oalkyl, or —CH₂N(alkyl)₂;
   R₉ is hydrogen or alkyl; and
   m is 0, 1, or 2.

In some embodiments, the present disclosure provides a compound having the structure of Formula (I-1a1):

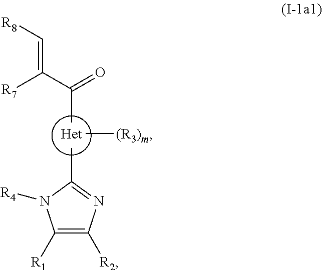

or a pharmaceutically acceptable salt, stereoisomer, or atropisomer thereof, wherein

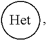

R₁, R₂, R₃, R₄, R₇, R₈, and m are as defined herein.

In some embodiments,

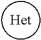

is:

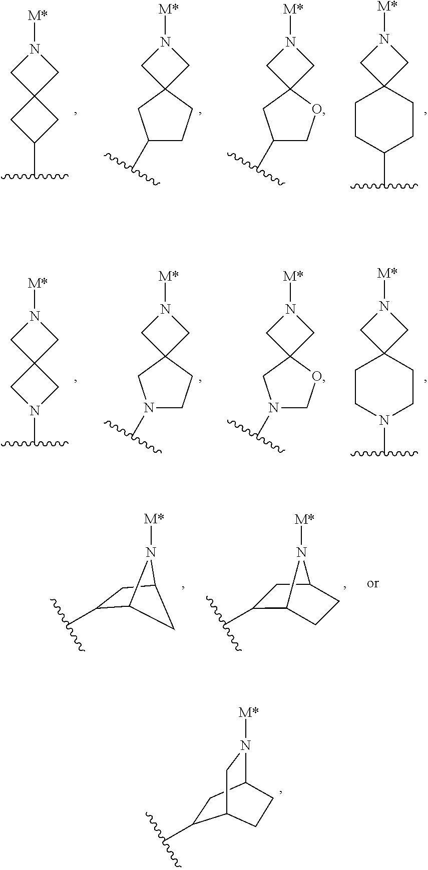

wherein M* represents the point of attachment to M and [symbol] represents the point of attachment to L.

In some embodiments, m is 0. In some embodiments, m is 1 or 2.

In some embodiments, the present disclosure provides a compound having the structure of Formula (I-1b2):

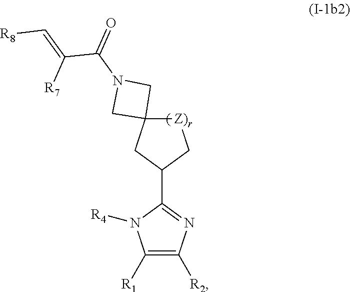

or a pharmaceutically acceptable salt, stereoisomer, or atropisomer thereof,
wherein:
R₁, R₂, R₄, R₇, and R₈ are as defined herein;
Z is CH₂ or O; and
r is 0 or 1.

In some embodiments, Z is CH₂.

In some embodiments, r is 0. In some embodiments, r is 1.

In some embodiments, the present disclosure provides a compound having the structure of Formula (I-1c1):

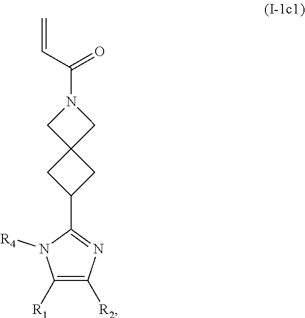

or a pharmaceutically acceptable salt, stereoisomer, or atropisomer thereof, wherein R₁, R₂, and R₄ are as defined herein.

In some embodiments, R₁ is:

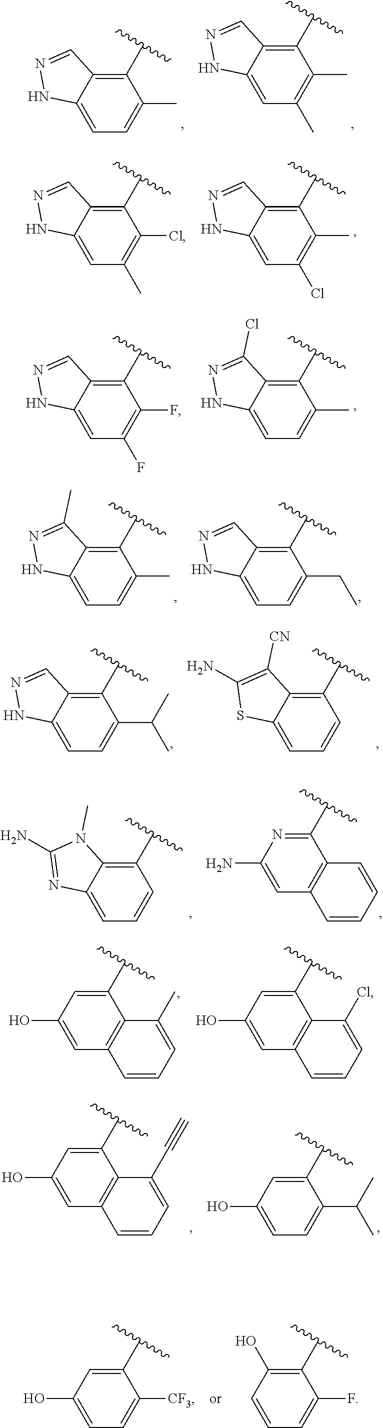

In some embodiments, R₂ is:

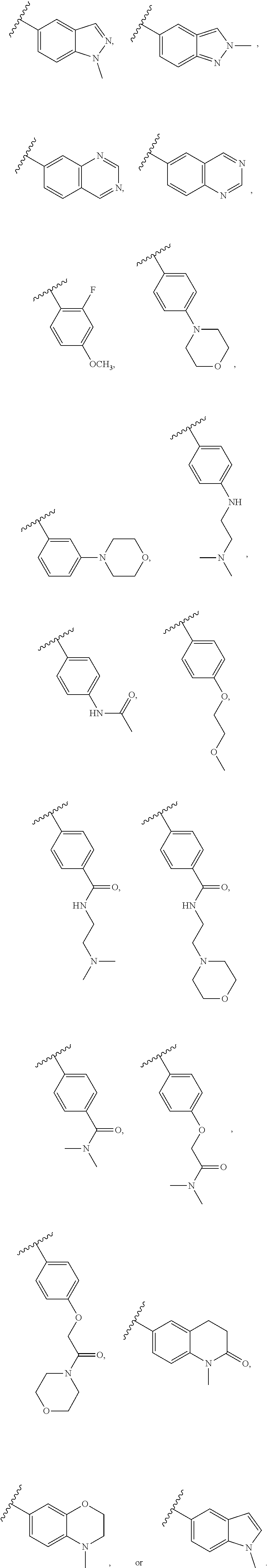

In some embodiments, R₄ is C₁₋₅alkyl. In some embodiments, R₄ is methyl.

In some embodiments, R₇ and R₈ are hydrogen.

In some embodiments, the present disclosure provides a pharmaceutical composition comprising a compound disclosed herein (e.g., a compound of Formula (I-1), Formula (I-2), Formula (I-1a), Formula (I-1a1), Formula (I-1b), Formula (I-1b1), Formula (I-1b2), Formula (I-1c), Formula (I-1c1), or Table A) and a pharmaceutically acceptable carrier or excipient.

In some embodiments, the present disclosure provides a method for treating a condition modulated by RAS proteins in a subject in need thereof, the method comprising, administering to the subject a therapeutically effect amount of a compound disclosed herein (e.g., a compound of Formula (I-1), Formula (I-2), Formula (I-1a), Formula (I-1a1), Formula (I-1b), Formula (I-1b1), Formula (I-1b2), Formula (I-1c), Formula (I-1c1), or Table A), or a pharmaceutical composition thereof.

In some embodiments, the condition modulated by RAS proteins is cancer. In some embodiments, the cancer is selected from the group consisting of sarcoma, melanoma, skin cancer, haematological tumors, lymphoma, carcinoma, and leukemia.

In some embodiments, the compound of the present disclosure is an inhibitor of KRAS proteins. In some embodiments, the compounds have comparable activity to existing treatments. In some embodiments, the compounds have improved activity compared to existing treatments. In some embodiments, the compounds have improved solubility compared to known inhibitors of KRAS proteins and existing therapies.

In some embodiments, the present disclosure provides compounds that exhibit reduced cytotoxicity in normal cells relative to prior art compounds and existing therapies.

In some embodiments, the present disclosure provides compounds having a therapeutically effective pharmacokinetic profile and a suitable duration of action following administration. In some embodiments, the metabolised fragment or fragments of the disclosed compound after absorption are classified as Generally Regarded As Safe (GRAS).

### Definitions

Certain terms employed in the specification, examples and claims are provided here. Unless defined otherwise, all technical and scientific terms used in this disclosure have the same meanings as commonly understood by one of ordinary skill in the art to which this disclosure belongs.

The term “alkyl” refers to a linear or branched hydrocarbon chain. For example, the term “C₁₋₆ alkyl” or “C₁₋₄-alkyl” refers to a linear or branched hydrocarbon chain containing 1, 2, 3, 4, 5, or 6 carbon atoms, for example methyl, ethyl, n-propyl, iso-propyl, n-butyl, sec-butyl, tert-butyl, n-pentyl and n-hexyl. Where an alkyl group is indicated as being C₀₋₄alkyl, then it should be appreciated that this represents the possibility for the alkyl unit to be absent or 1, 2, 3, or 4 carbon atoms in length. Unless stated otherwise specifically in the specification, the alkyl groups may be optionally substituted by one or more substituents. Non-limiting examples of suitable substituents are described below. In some embodiments, substituents for the alkyl group include halogen, e.g. fluorine, chlorine, bromine and iodine, OH, C₁₋₆ alkoxy, and amino.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

The term “alkoxy” refers to an alkyl group which is attached to a molecule via oxygen. For example, the term “C₁₋₆ alkoxy” refers to an alkyl group which is attached to a molecule via oxygen. This includes moieties where the alkyl part may be linear or branched and may contain 1, 2, 3, 4, 5, or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy. Unless stated otherwise specifically in the specification, the alkyl moiety of the alkoxy group may be optionally substituted by one or more substituents. Non-limiting examples of suitable substituents are described below. In some embodiments, substituents for the alkyl group include halogen, e.g. fluorine, chlorine, bromine and iodine, OH, C₁₋₆ alkoxy, and amino.

The term “haloalkyl” refers to a hydrocarbon chain substituted with at least one halogen atom independently chosen at each occurrence, for example fluorine, chlorine, bromine and iodine. For example, the term “C₁₋₆ haloalkyl” refers to a linear or branched hydrocarbon chain containing 1, 2, 3, 4, 5 or 6 carbon atoms substituted with at least one halogen. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. Unless stated otherwise specifically in the specification, a haloalkyl chain can be optionally substituted.

The term “alkenyl” refers to a branched or linear hydrocarbon chain containing at least one double bond. For example, the term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl. Unless stated otherwise specifically in the specification, an alkenyl can be optionally substituted.

The term “alkynyl” refers to a branched or linear hydrocarbon chain containing at least one triple bond. For example, the term “C₂₋₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Unless stated otherwise specifically in the specification, alkynyl can be optionally substituted.

The term “heteroalkyl” refers to a branched or linear hydrocarbon chain containing at least one heteroatom selected from N, O and S positioned between any carbon in the chain or at an end of the chain. For example, the term “C₁₋₆ heteroalkyl” refers to a branched or linear hydrocarbon chain containing 1, 2, 3, 4, 5, or 6 carbon atoms and at least one heteroatom selected from N, O and S positioned between any carbon in the chain or at an end of the chain. For example, the hydrocarbon chain may contain one or two heteroatoms. The C₁₋₆ heteroalkyl may be bonded to the rest of the molecule through a carbon or a heteroatom. For example, the “C₁₋₆ heteroalkyl” may be C₁₋₆ N-alkyl, C₁₋₆ N,N-alkyl, or C₁₋₆ O-alkyl. Unless stated otherwise specifically in the specification, a heteroalkyl can be optionally substituted.

The term “carbocyclic” or “carbocyclic ring” refers to a saturated or unsaturated carbon containing ring system. A “carbocyclic” system may be monocyclic or a fused polycyclic ring system, for example, bicyclic or tricyclic. A “carbocyclic” moiety may contain from 3 to 14 carbon atoms, for example, 3 to 8 carbon atoms in a monocyclic system and 7 to 14 carbon atoms in a polycyclic system. “Carbocyclic” encompasses cycloalkyl moieties, cycloalkenyl moieties, aryl ring systems and fused ring systems including an aromatic portion. Unless stated otherwise specifically in the specification, a carbocyclic ring can be optionally substituted.

The term “heterocyclic” or “heterocyclic ring” refers to a saturated or unsaturated ring system containing at least one heteroatom selected from N, O and S. A “heterocyclic” system may contain 1, 2, 3 or 4 heteroatoms, for example 1 or 2 heteroatoms. A “heterocyclic” system may be monocyclic or a fused polycyclic ring system, for example, bicyclic or tricyclic. A “heterocyclic” moiety may contain from 3 to 14 atoms, for example, 3 to 8 atoms in a monocyclic system and 7 to 14 atoms in a polycyclic system. “Heterocyclic” encompasses heterocycloalkyl moieties, heterocycloalkenyl moieties and heteroaryl moieties. For example, the heterocyclic group may be: oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran. Unless stated otherwise specifically in the specification, a heterocyclic ring can be optionally substituted.

The term “heterocycloalkyl” refers to a saturated hydrocarbon ring system containing carbon atoms and at least one heteroatom within the ring selected from N, O and S. For example, there may be 1, 2 or 3 heteroatoms, optionally 1 or 2 heteroatoms. The “heterocycloalkyl” may be bonded to the rest of the molecule through any carbon atom or heteroatom. The “heterocycloalkyl” may have one or more, e.g. one or two, bonds to the rest of the molecule: these bonds may be through any of the atoms in the ring. For example, the “heterocycloalkyl” may be a “3- to 8-membered heterocycloalkyl”. The term “3- to 8-membered heterocycloalkyl” refers to a saturated hydrocarbon ring system containing 3, 4, 5, 6, 7 or 8 atoms, including at least one heteroatom within the ring selected from N, O and S. For example, there may be 1, 2 or 3 heteroatoms, optionally 1 or 2. The “3- to 8-membered heterocycloalkyl” may be bonded to the rest of the molecule through any carbon atom or heteroatom. The “3- to 8-membered heterocycloalkyl” may have one or more, e.g. one or two, bonds to the rest of the molecule: these bonds may be through any of the atoms in the ring. For example, the “3- to 8-membered heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “heterocycloalkenyl” refers to an unsaturated hydrocarbon ring system that is not aromatic, containing carbon atoms and at least one heteroatom within the ring selected from N, O and S. For example, there may be 1, 2 or 3 heteroatoms, optionally 1 or 2 heteroatoms. The “heterocycloalkenyl” may be bonded to the rest of the molecule through any carbon atom or heteroatom. The “heterocycloalkenyl” may have one or more, e.g. one or two, bonds to the rest of the molecule: these bonds may be through any of the atoms in the ring. For example, the “heterocycloalkenyl” may be a “3- to 8-membered heterocycloalkenyl”. The term “3- to 8-membered heterocycloalkenyl” refers to a saturated hydrocarbon ring system containing 3, 4, 5, 6, 7 or 8 atoms, wherein at least one of the atoms is a heteroatom within the ring selected from N, O and S. The “heterocycloalkenyl” may be tetrahydropyridine, dihydropyran, dihydrofuran, pyrroline.

The term “aromatic” when applied to a substituent as a whole means a single ring or polycyclic ring system with 4n+2 electrons in a conjugated π system within the ring or ring system where all atoms contributing to the conjugated π system are in the same plane.

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl or naphthyl. Unless stated otherwise specifically in the specification, the “aryl” can be optionally substituted.

The term “heteroaryl” refers to an aromatic hydrocarbon ring system with at least one heteroatom within a single ring or within a fused ring system, selected from O, N and S. The ring or ring system has 4n+2 electrons in a conjugated π system where all atoms contributing to the conjugated π system are in the same plane. For example, the “heteroaryl” may be imidazole, thiene, furane, thianthrene, pyrrole, benzimidazole, pyrazole, pyrazine, pyridine, pyrimidine and indole. Unless stated otherwise specifically in the specification, the “heteroaryl” can be optionally substituted.

The term “halo” or “halide”, as used herein, refers to F, Cl, Br and I. In some embodiments, halo refers to fluoride or chloride.

By “acyl” is meant an organic radical derived from, for example, an organic acid by the removal of the hydroxyl group, e.g. A radical having the formula r-c(o)-, where r may be selected from h, c₁₋₆ alkyl, c₃₋₈ cycloalkyl, phenyl, benzyl or phenethyl group, e.g. r is h or c₁₋₃ alkyl. In one embodiment acyl is alkyl-carbonyl. Examples of acyl groups include, but are not limited to, formyl, acetyl, propionyl and butyryl. A particular acyl group is acetyl.

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different. The substituent(s) may be selected from: OH, NHR^{6d}, —N(R^{6d})₂, amidino, guanidino, hydroxyguanidino, formamidino, isothioureido, ureido, mercapto, C(O)H, acyl, acyloxy, carboxy, sulfo, sulfamoyl, carbamoyl, cyano, azo, nitro, halo, C₁₋₆ alkyl, C₁₋₆ alkoxy, C₁₋₆ haloalkyl, C₃₋₈ cycloalkyl, C₂₋₆ alkenyl, C₂₋₆ alkynyl, aryl, heteroaryl or alkaryl. Where the group to be substituted is an alkyl group the substituent may be ═O. Where the moiety is substituted with two or more substituents and two of the substituents are adjacent the adjacent substituents may form a C₄₋₈ ring along with the atoms of the moiety on which the substituents are substituted, wherein the C₄₋₈ ring is a saturated or unsaturated hydrocarbon ring with 4, 5, 6, 7, or 8 carbon atoms or a saturated or unsaturated hydrocarbon ring with 4, 5, 6, 7, or 8 carbon atoms and 1, 2 or 3 heteroatoms.

Substituents are only present at positions where they are chemically possible, the person skilled in the art being able to decide (either experimentally or theoretically) without inappropriate effort which substitutions are chemically possible, and which are not.

Throughout the description and claims of this specification, the words “comprise” and “contain” and variations thereof mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. Theis disclosure is not restricted to the details of any foregoing embodiments. This disclosure extends to any novel one, or any novel combination, of the features disclosed in this specification (including any accompanying claims, abstract and drawings), or to any novel one, or any novel combination, of the steps of any method or process so disclosed.

### DETAILED DESCRIPTION

### Compounds of the Disclosure

In one aspect, the present disclosure provides a compound of Formula (I-1) or Formula (I-2):

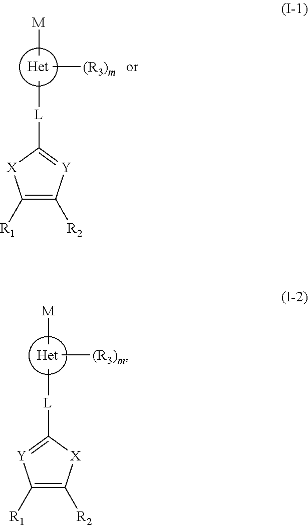

or a pharmaceutically acceptable salt, stereoisomer, or atropisomer thereof,
wherein:
X is N—R₄, O, or S;
Y is N or CH;

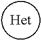

is a nitrogen-containing heterocyclyl;
L is a bond, alkylene, alkenylene, alkynylene, —C(O)—, or —S(O)₂—;
R₁ and R₂ are each independently aryl or heteroaryl;
R₃ is hydrogen, halogen, alkyl, hydroxy, alkoxy, —CN, —C(O)OR₅, or —S(O)₂NR₅R₆; or two R₃ groups attached to the same carbon atom form an oxo, cycloalkyl, or heterocyclyl; or two R₃ groups taken together with the carbon atoms to which they are attached form a cycloalkyl or heterocyclyl;
R₄ is alkyl, cycloalkyl, alkylenecycloalkyl, heterocyclyl, or alkyleneheterocyclyl;
R₅ and R₆ are each independently alkyl, cycloalkyl, heterocyclyl, aryl, heteroaryl, alkylenecycloalkyl, alkyleneheterocyclyl, alkylenearyl, or alkyleneheteroaryl;
M is

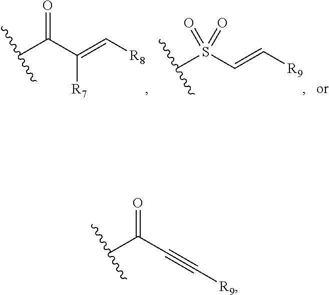

wherein
   R₇ is hydrogen, halogen, C₁-C₃alkyl, —C(O)—C₁-C₃alkyl, or —CN;
   R₈ is H, alkyl, —CF₃, —CHF₂, —CH₂F, —CH₂—Oalkyl, or —CH₂N(alkyl)₂;
   R₉ is hydrogen or alkyl; and
   m is 0, 1, or 2.

In some embodiments, the present disclosure provides a compound of Formula (I-1):

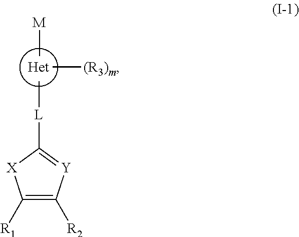

or a pharmaceutically acceptable salt, stereoisomer, or atropisomer thereof,
wherein:

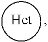

R₁, R₂, R₃, L, M, X, Y, and m are as defined herein.

In some embodiments,

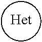

is a 4-12 membered nitrogen-containing heterocyclic ring. In some embodiments,

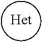

is a 5-10-membered nitrogen-containing heterocyclic ring. In some embodiments, the nitrogen-containing heterocyclic ring comprises 1-3 heteroatoms selected from the group consisting of N, O, and S. In some embodiments, the nitrogen-containing heterocyclic ring comprises 2 heteroatoms selected from the group consisting of N, O, and S. In some embodiments, the nitrogen-containing heterocyclic ring comprises 1 N atom. In some embodiments,

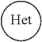

is a spirocyclic nitrogen-containing heterocyclic ring. In some embodiments,

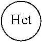

is a spirocyclic nitrogen-containing heterocyclyl comprising 1-3 heteroatoms. In some embodiments,

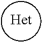

is a spirocyclic nitrogen-containing heterocyclyl comprising 1 or 2 heteroatoms. In some embodiments,

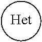

is a spirocyclic nitrogen-containing heterocyclyl comprising 1 heteroatom. In some embodiments,

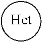

is optionally substituted. In some embodiments,

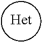

is optionally substituted with one or more R³ groups as defined herein.

In some embodiments,

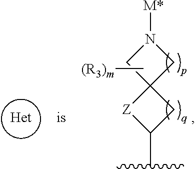

wherein M* represents the point of attachment to M; Z is O or CH₂; p and q are each independently 1-3; and R₃ and m are as defined herein.

In some embodiments,

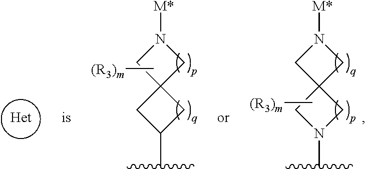

wherein M* represents the point of attachment to M; p and q are each independently 1-3; and R₃ and m are as defined herein.

In some embodiments,

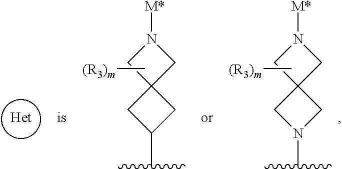

wherein M* represents the point of attachment to M; and R₃ and m are as defined herein.

In some embodiments,

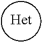

is:

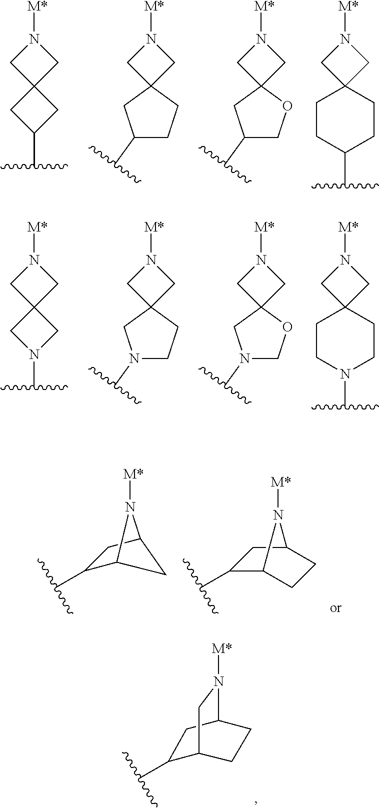

wherein M* represents the point of attachment to M and [symbol] represents the point of attachment to L.

In some embodiments,

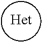

is

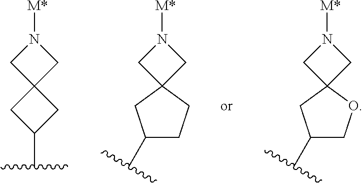

wherein M* represents the point of attachment to M and [symbol] represents the point of attachment to L.

In some embodiments,

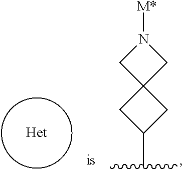

wherein M* represents the point of attachment to M and [symbol] represents the point of attachment to L.

In some embodiments,

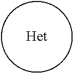

is optionally substituted with one or more R₃ substituents as defined herein. In some embodiments,

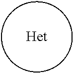

is optionally substituted with one or more R₃ substituents selected from the group consisting of hydrogen, halogen, alkyl, hydroxy, alkoxy, —CN, —C(O)OR₅, or —S(O)₂NR₅R₆. In some embodiments, two R₃ groups attached to the same carbon atom form an oxo, cycloalkyl, or heterocyclyl. In some embodiments, two R₃ groups taken together with the carbon atoms to which they are attached form a cycloalkyl or heterocyclyl. In some embodiments, the cycloalkyl is a C₃₋₆cycloalkyl. In some embodiments, the heterocyclyl is a 3- to 8-membered heterocyclyl. In some embodiments, the heterocyclyl is a 3- to 8-membered heterocyclyl comprising 1 or 2 heteroatoms selected from the group consisting of N, O, and S.

In some embodiments, R₁ is a phenol, napthol, or heteroaryl. In some embodiments, R₁ is a phenol, napthol, or heteroaryl comprising 1, 2, or 3 heteroatoms selected from the group consisting of N, O, and S. In some embodiments, the heteroaryl is a monocyclic or bicyclic heteroaryl. In some embodiments, the bicyclic heteroaryl is a fused bicyclic heteroaryl. In some embodiments, the bicyclic heteroaryl comprises a 5,6- or 6,6-fused bicyclic ring system.

In some embodiments, R₁ is:

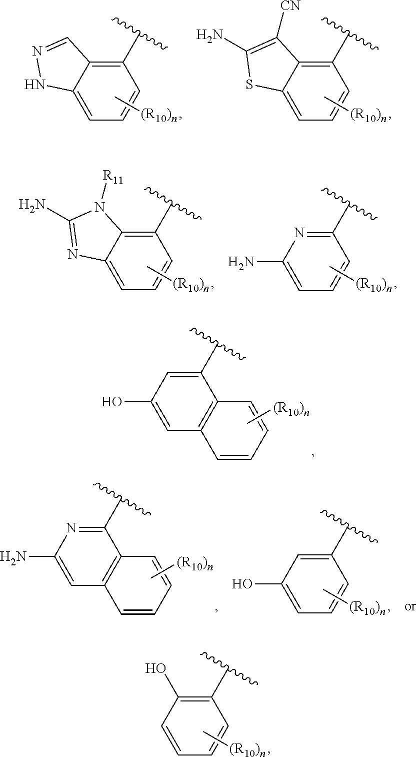

wherein:
R₁₀ is each independently halogen, alkyl, alkenyl, alkynyl, alkoxy, —CN, or cycloalkyl;
R₁₁ is H, alkyl, or cycloalkyl; and
n is 0, 1, 2, or 3.

In some embodiments, R₁ is:

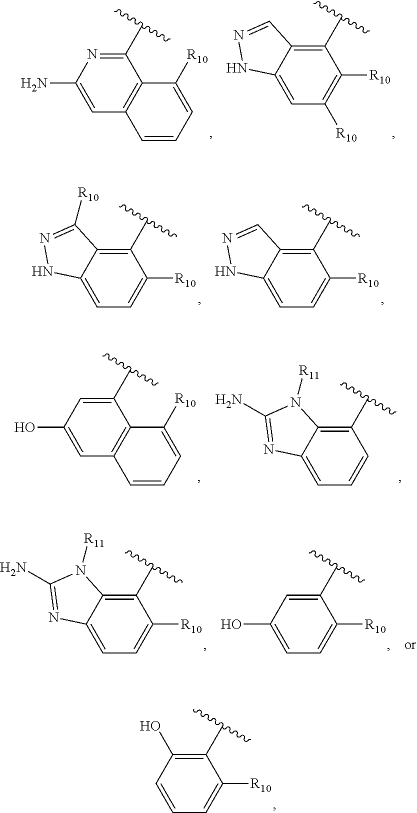

wherein:
R₁₀ is each independently halogen, alkyl, alkenyl, alkynyl, alkoxy, —CN, or cycloalkyl;
R₁₁ is H, alkyl, or cycloalkyl; and
n is 0, 1, 2, or 3.

In some embodiments, R₁₀ is each independently halogen, alkyl, alkenyl, alkynyl, or alkoxy. In some embodiments, R₁₀ is each independently halogen, alkyl, cycloalkyl, or alkynyl. In some embodiments, R₁₀ is each independently halogen, alkyl, or alkynyl. In some embodiments, R₁₀ is each independently halogen or alkyl. In some embodiments, the halogen is F or Cl. In some embodiments, the alkyl is a C₁₋₅alkyl. In some embodiments, alkenyl is a C₁₋₅alkenyl. In some embodiments, the alkynyl is a C₁₋₅alkynyl. In some embodiments, the cycloalkyl is a C₃₋₆-cycloalkyl. In some embodiments, the alkoxy is a C₁₋₅alkoxy. In some embodiments, R₁₀ is each independently halogen, C₁₋₅alkyl, C₁₋₅alkenyl, C₁₋₅alkynyl, C₁₋₅alkoxy, —CN, or C₃₋₆-cycloalkyl. In some embodiments, R₁₀ is each independently halogen, C₁₋₅alkyl, C₁₋₅alkenyl, C₁₋₅alkynyl, C₁₋₅alkoxy, —CN, or C₃₋₆-cycloalkyl. In some embodiments, R₁₀ is each independently halogen, C₁₋₅alkyl, C₁₋₅alkynyl, or C₃₋₆-cycloalkyl. In some embodiments, R₁₀ is each independently halogen, C₁₋₅alkyl, or C₁₋₅alkynyl.

In some embodiments, R₁₁ is H, alkyl, cycloalkyl, alkylenecycloalkyl, or alkyleneheterocyclyl. In some embodiments, R₁₁ is H, C₁₋₅alkyl, C₃₋₆-cycloalkyl, —CH₂—(C₃₋₅cycloalkyl), or —CH₂-heterocycyl. In some embodiments, R₁₁ is H, C₁₋₅alkyl, C₃₋₆-cycloalkyl, or —CH₂—(C₃₋₆-cycloalkyl). In some embodiments, R₁₁ is H, C₁₋₅alkyl, or C₃₋₆-cycloalkyl. In some embodiments, R₁₁ is C₁₋₅alkyl or C₃₋₆-cycloalkyl. In some embodiments, R₁₁ is C₁₋₅alkyl. In some embodiments, R₁₁ is methyl or isopropyl. In some embodiments, R₁₁ is methyl.

In some embodiments, n is 0, 1, or 2. In some embodiments, n is 0 or 1. In some embodiments, n is 1, 2, or 3. In some embodiments, n is 1 or 2. In some embodiments, n is 2 or 3.

In some embodiments, R₁ is:

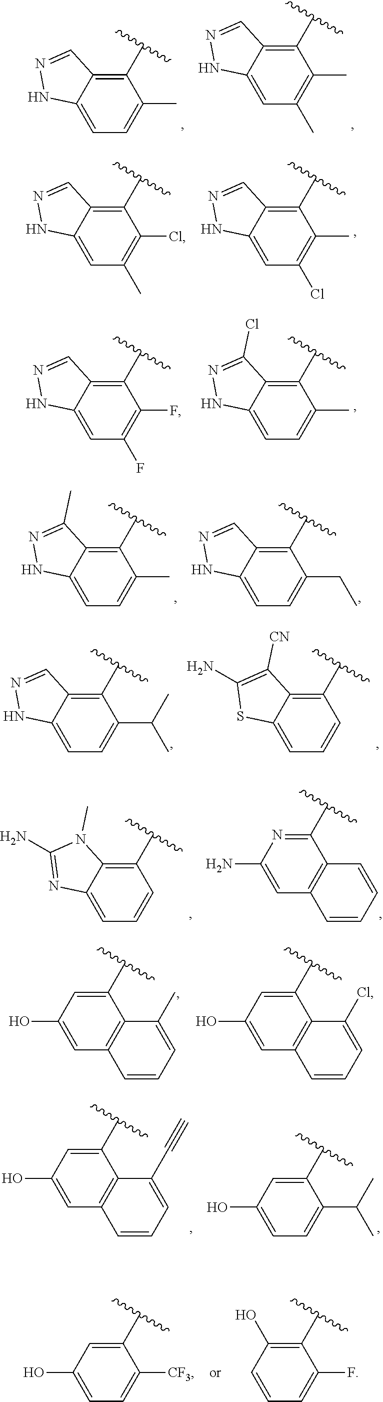

In some embodiments, R₂ is a substituted phenyl or nitrogen-containing heteroaryl. In some embodiments, the nitrogen-containing heteroaryl is a 5- to 14-membered heteroaryl. In some embodiments, the nitrogen-containing heteroaryl is a fused bicyclic heteroaryl ring. In some embodiments, the fused bicyclic heteroaryl ring comprises 1, 2, or 3 heteroatoms selected from the group consisting of N, O, and S. In some embodiments, the fused bicyclic heteroaryl ring comprises 1-3 nitrogen atoms. In some embodiments, the fused bicyclic heteroaryl comprises a 5,6- or 6,6-fused ring system.

In some embodiments, R₂ is:

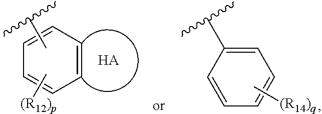

wherein:

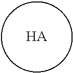

is a 5- or 6-membered nitrogen-containing heteroaryl ring;
R₁₂ is each independently halogen or alkyl; and
R₁₄ is halogen, alkyl, alkoxy, —O—(C₂₋₄alkylene)-O-alkyl, —O—(C₁₋₃alkylene)-C(O)NR₁₅R₁₆, —CO₂alkyl, —C(O)NR₁₅R₁₆, —C(O)NH—(C₂₋₄alkylene)-NR₁₅R₁₆, —N(H)C(O)alkyl, —NH—(C₂₋₄alkylene)-NR₁₅R₁₆, cycloalkyl, heterocyclyl, aryl, or heteroaryl, or two R₁₄ groups taken together with the carbon atoms to which they are attached form a heterocyclyl;
R₁₅ and R₁₆ are each independently H or alkyl, or an R₁₅ and R₁₆ taken together with the nitrogen atom to which they are attached form a heterocyclyl;
p is 0, 1, or 2; and
q is 0, 1 or 2.

In some embodiments, R₂ is:

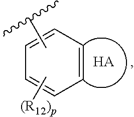

wherein

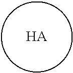

is a 5- or 6-membered nitrogen-containing heteroaryl ring;
R₁₂ is each independently halogen or alkyl; and
p is 0, 1, or 2.

In some embodiments,

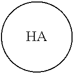

is a pyridine, pyrimidine, pyridazine, pyrazine, pyrrazole, imidazole, or pyrrole ring.

In some embodiments, R₂ is:

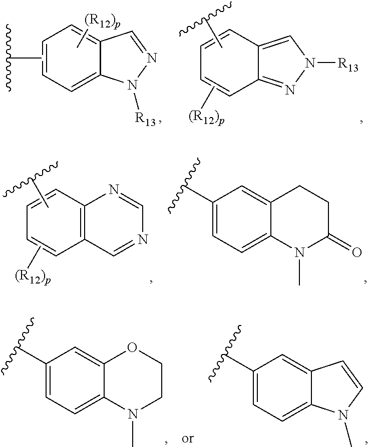

wherein:
R₁₂ is each independently halogen, alkyl, or cycloalkyl;
R₁₃ is alkyl or cycloalkyl; and
p is 0, 1, or 2.

In some embodiments, R₁₂ is H, alkyl, or haloalkyl. In some embodiments, R₁₂ is H or alkyl. In some embodiments, R₁₂ is each independently halogen, C₁₋₅alkyl, C₁₋₅haloalkyl, or C₃₋₆cycloalkyl. In some embodiments, R₁₂ is each independently halogen, C₁₋₅alkyl, or C₃₋₆cycloalkyl. In some embodiments, R₁₂ is halogen, C₁₋₅alkyl, or C₁₋₅haloalkyl. In some embodiments, the halogen is F, Cl, or Br. In some embodiments, R₁₂ is F, Cl, or C₁₋₅alkyl. In some embodiments, R₁₂ is F, Cl, —CH₃, —CH₂CH₃, —CH(CH₃)₂, —CH₂CH(CH₃)₂, —C(CH₃)₃, or —CF₃.

In some embodiments, R₁₃ is H, alkyl, cycloalkyl, or —CH₂-cycloalkyl. In some embodiments, R₁₃ is alkyl, cycloalkyl, or —CH₂-cycloalkyl. In some embodiments, R₁₃ is C₁₋₅alkyl, C₃₋₆cycloalkyl, or —CH₂—(C₃₋₆cycloalkyl). In some embodiments, R₁₃ is alkyl. In some embodiments, R₁₃ is C₁₋₅alkyl or C₃₋₆cycloalkyl. In some embodiments, R₁₃ is C₁₋₅alkyl. In some embodiments, R₁₃ is —CH₃, —CH₂CH₃, or —CH(CH₃)₂.

In some embodiments, p is 0 or 1. In some embodiments, p is 1 or 2. In some embodiments, p is 0. In some embodiments, p is 1. In some embodiments, p is 2.

In some embodiments, R₂ is:

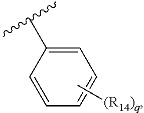

wherein:
R₁₄ is halogen, alkyl, alkoxy, —O—(C₂₋₄alkylene)-O-alkyl, —O—(C₁₋₃alkylene)-C(O)NR₁₅R₁₆, —CO₂alkyl, —C(O)NR₁₅R₁₆, —C(O)NH—(C₂₋₄alkylene)-NR₁₅R₁₆, —N(H)C(O)alkyl, —NH—(C₂₋₄alkylene)-NR₁₅R₁₆, cycloalkyl, heterocyclyl, —CH₂— heterocyclyl, aryl, or heteroaryl, or two R₁₄ groups taken together with the carbon atoms to which they are attached form a heterocyclyl or heteroaryl; R₁₅ and R₁₆ are each independently H or alkyl, or an R₁₅ and R₁₆ taken together with the nitrogen atom to which they are attached form a heterocyclyl; and q is 0, 1 or 2.

In some embodiments, R₁₄ is halogen, C₁₋₅alkyl, C₁₋₅alkoxy, —O—(C₂₋₄alkylene)-O—C₁₋₅alkyl, —O—(C₁₋₃alkylene)-C(O)NR₁₅R₁₆, —CO₂C₁₋₅alkyl, —C(O)NR₁₅R₁₆, —C(O)NH—(C₂₋₄alkylene)-NR₁₅R₁₆, —N(H)C(O)C₁₋₅alkyl, —NH—(C₂₋₄alkylene)-NR₁₅R₁₆, C₃₋₆cycloalkyl, 3- to 8-membered heterocyclyl, —CH₂-heterocyclyl, phenyl, naphthyl, or 5- to 6-membered heteroaryl (e.g., pyridinyl, pyrimidinyl, pyrazinyl, pyridazinyl, oxazolyl, thiazolyl, oxadiazolyl, thiadiazolyl, imidazolyl, or triazolyl). In some embodiments, the heterocyclyl is morpholino, thiomorpholino, or N-alkylpiperazinyl (e.g., N-methylpiperazinyl, N-ethylpiperazinyl, and N-isopropylpiperazinyl. In some embodiments, R₁₄ is heterocyclyl. In some embodiments, R₁₄ is 6-membered heterocyclyl. In some embodiments, R₁₄ is 6-membered heterocyclyl. In some embodiments, R₁₄ is 6-membered heterocyclyl having 1 or 2-heteroatoms selected from the group consisting of N, O, and S. In some embodiments, R₁₄ is morpholino, thiomorpholino, or N-methylpiperazinyl. In some embodiments, R₁₄ is morpholino or thiomorpholino. In some embodiments, R₁₄ is morpholino. In some embodiments, R₁₄ is

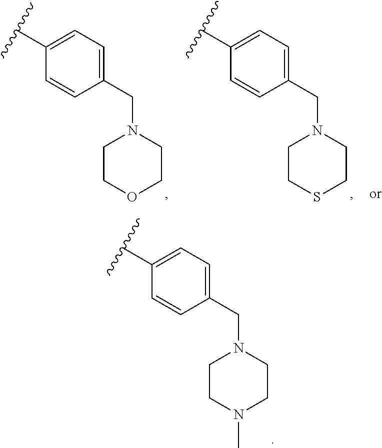 [SMILES: *c1ccc(CN2CCOCC2)cc1 ; *c1ccc(CN2CCSCC2)cc1 ; *c1ccc(CN2CCN(C)CC2)cc1]

In some embodiments, R₁₄ is heteroaryl. In some embodiments, R₁₄ is a 5-membered heteroaryl. In some embodiments, R₁₄ is a 5-membered heteroaryl having 1-3 heteroatoms selected from the group consisting of N, O, and S. In some embodiments, R₁₄ is a 5-membered heteroaryl having 1 or 2 N atoms. In some embodiments, two R₁₄ groups taken together with the carbon atoms to which they are attached form a heteroaryl. In some embodiments, the heteroaryl is a 5-membered heteroaryl. In some embodiments, the 5-membered heteroaryl has 1-3 heteroatoms selected from the group consisting of N, O, and S. In some embodiments, the 5-membered heteroaryl has 1 or 2 N atoms. In some embodiments, the 5-membered heteroaryl is an optionally substituted imidazole or pyrazole.

In some embodiments, q is 0 or 1. In some embodiments, q is 1 or 2. In some embodiments, q is 0. In some embodiments, q is 1. In some embodiments, q is 2.

In some embodiments, R₂ is:

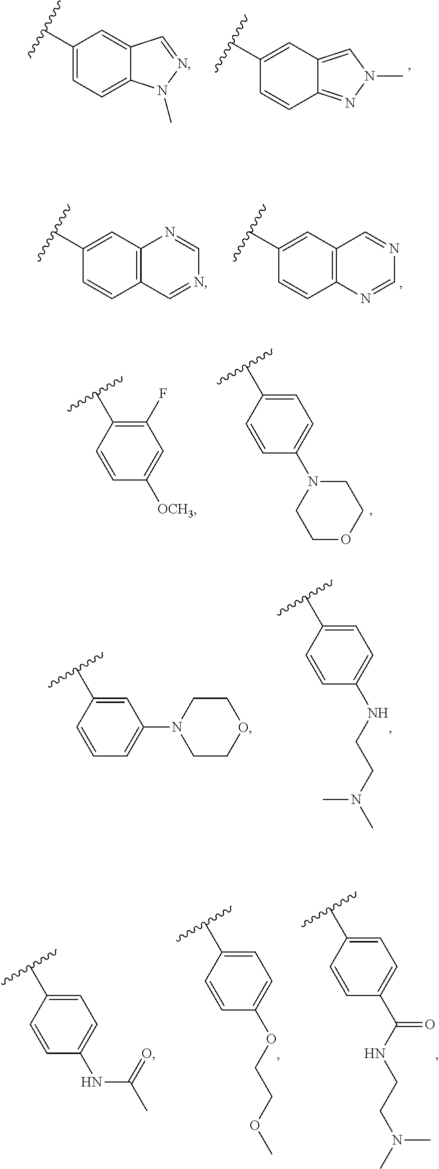

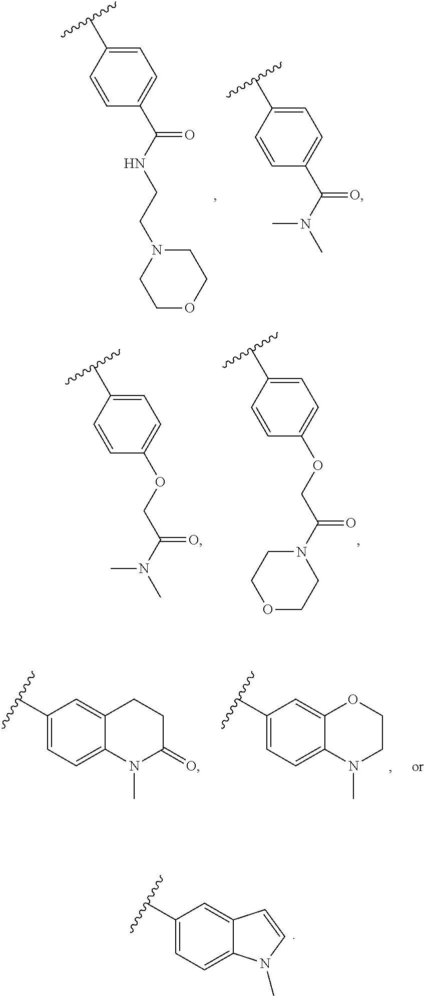

In some embodiments, R₃ is selected from the group consisting of hydrogen, halogen, alkyl, hydroxy, alkoxy, —CN, —C(O)OR₅, or —S(O)₂NR₅R₆. In some embodiments, two R₃ groups attached to the same carbon atom form an oxo, cycloalkyl, or heterocyclyl. In some embodiments, two R₃ groups attached to the same carbon atom form an oxo. In some embodiments, two R₃ groups taken together with the carbon atoms to which they are attached form a cycloalkyl or heterocyclyl. In some embodiments, the cycloalkyl is a C₃₋₆cycloalkyl. In some embodiments, the heterocyclyl is a 3- to 8-membered heterocyclyl. In some embodiments, the heterocyclyl is a 3- to 8-membered heterocyclyl comprising 1 or 2 heteroatoms selected from the group consisting of N, O, and S. In some embodiments, R₃ is oxo, hydroxy, —CN, C₁₋₅alkyl, or C₁₋₅alkoxy.

In some embodiments, R₅ and R₆ are each independently C₁₋₅alkyl, C₃₋₆cycloalkyl, 3- to 8-membered heterocyclyl, phenyl, 5- or 6-membered heteroaryl, —CH₂—(C₃₋₆cycloalkyl), —CH₂-heterocyclyl, —CH₂-aryl, or —CH₂-heteroaryl. In some embodiments, R₅ and R₆ are each independently C₁₋₅alkyl, C₃₋₆cycloalkyl, —CH₂—(C₃₋₆cycloalkyl). In some embodiments, R₅ and R₆ are each independently C₁₋₅alkyl or C₃₋₆cycloalkyl.

In some embodiments, m is 0 or 1. In some embodiments, m is 0. In some embodiments, m is 1. In some embodiments, m is 2.

In some embodiments, X is N—R₄.

In some embodiments, Y is N. In some embodiments, Y is CH.

In some embodiments, X is N—R₄ and Y is N or CH. In some embodiments, X is N—R₄ and Y is N.

In some embodiments, R₄ is alkyl, haloalkyl, alkylenecycloalkyl, or cycloalkyl. In some embodiments, R₄ is alkyl, alkylenecycloalkyl, or cycloalkyl. In some embodiments, R₄ is alkyl or alkylenecycloalkyl. In some embodiments, the alkyl is a C₁₋₅alkyl. In some embodiments, the cycloalkyl is a C₃₋₆cycloalkyl. In some embodiments, the alkylene is a C₁₋₃alkylene. In some embodiments, the alkylene is a methylene. In some embodiments, R₄ is alkyl or cycloalkyl. In some embodiments, R₄ is C₁₋₅alkyl, C₁₋₅haloalkyl, —CH₂—(C₃₋₆cycloalkyl), or C₃₋₆cycloalkyl. In some embodiments, R₄ is C₁₋₅alkyl, —CH₂—(C₃₋₆cycloalkyl), or C₃₋₆cycloalkyl. In some embodiments, R₄ is C₁₋₅alkyl or C₃₋₆cycloalkyl. In some embodiments, R₄ is C₁₋₅alkyl. In some embodiments, R₄ is methyl, ethyl, isopropyl, cyclopropyl, —CH₂CF₃, or —CH₂CF₂. In some embodiments, R₄ is methyl.

In some embodiments, M is:

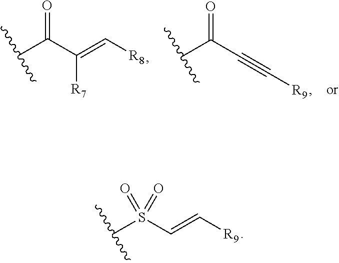

In some embodiments, M is

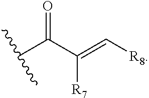

In some embodiments, M is

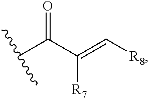

wherein R⁷ and R⁸ are H. Accordingly, in some embodiments, M is

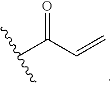 [SMILES: *C(=O)C=C]

In some embodiments, M is

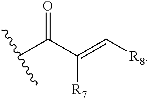

wherein R⁷ is an electron withdrawing group (e.g., halogen, haloalkyl, ester, and the like) and R⁸ is H. In some embodiments, M is

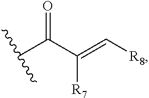

wherein R⁷ is F and R⁸ is H.

In some embodiments, R₇ is hydrogen, F, methyl, —C(O)CH₃, or —CN. In some embodiments, R₇ is H or F. In some embodiments, R₇ is H.

In some embodiments, R₈ is H, C₁₋₅alkyl, —CF₃, —CHF₂, —CH₂F, —CH₂—OC₁₋₅alkyl, or —CH₂N(CH₃)₂. In some embodiments, R₈ is H.

In some embodiments, R₉ is hydrogen or C₁₋₅alkyl. In some embodiments, R₉ is hydrogen or methyl.

In some embodiments, R₇ and R₈ are hydrogen.

In some embodiments, L is selected from the group consisting of a bond, alkylene, —C(O)—, —S(O)₂—, and —O—. In some embodiments, L is selected from the group consisting of a bond, alkylene, —C(O)—, or —S(O)₂—. In some embodiments, L is a bond, alkylene, or —C(O)—. In some embodiments, L is a bond or alkylene. In some embodiments, the alkylene is a C₁₋₃alkylene. In some embodiments, the alkylene is a methylene (i.e., —CH₂—) In some embodiments, L is a bond.

In some embodiments, the present disclosure provides a compound having the structure of Formula (I-1a):

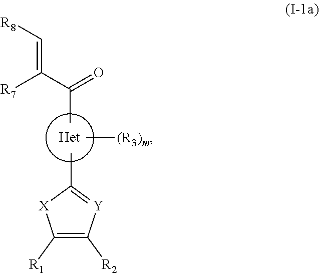

or a pharmaceutically acceptable salt, stereoisomer, or atropisomer thereof, wherein

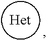

X, Y, R₁, R₂, R₃, R₄, R₇, R₈, and m are as defined herein.

In some embodiments, the present disclosure provides a compound having the structure of Formula (I-1a1):

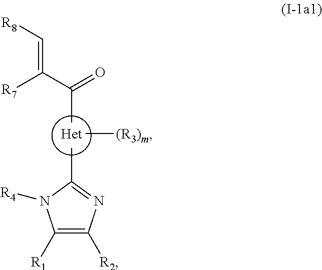

or a pharmaceutically acceptable salt, stereoisomer, or atropisomer thereof, wherein

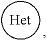

R₁, R₂, R₃, R₄, R₇, R₈, and m are as defined herein.

In some embodiments, the present disclosure provides a compound having the structure of Formula (I-1b):

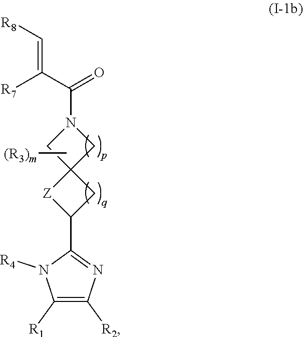

or a pharmaceutically acceptable salt, stereoisomer, or atropisomer thereof,
wherein:
R₁, R₂, R₃, R₄, R₇, R₈, and m are as defined herein;
Z is CH₂ or O; and
p and q are each independently 1-3.

In some embodiments, the present disclosure provides a compound having the structure of Formula (I-1b1):

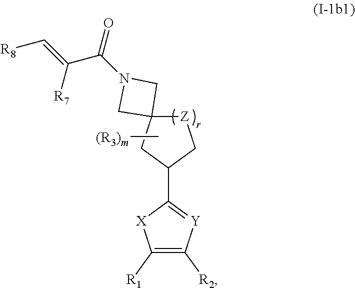

or a pharmaceutically acceptable salt, stereoisomer, or atropisomer thereof,
wherein:
X, Y, R₁, R₂, R₃, R₇, R₈, and m are as defined herein;
Z is CH₂ or O; and
r is 0 or 1.

In some embodiments, the present disclosure provides a compound having the structure of Formula (I-1b2):

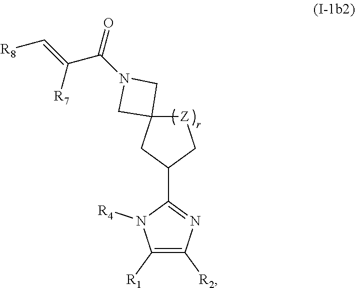

or a pharmaceutically acceptable salt, stereoisomer, or atropisomer thereof,
wherein:
R₁, R₂, R₄, R₇, and Rₐ are as defined herein;
Z is CH₂ or O; and
r is 0 or 1.

In some embodiments, Z is CH₂.

In some embodiments, r is 0. In some embodiments, r is 1.

In some embodiments, the present disclosure provides a compound having the structure of Formula (I-1c):

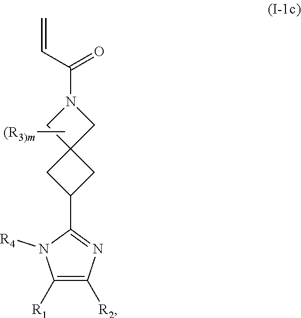

or a pharmaceutically acceptable salt, stereoisomer, or atropisomer thereof, wherein R₁, R₂, R₃, R₄, and m are as defined herein.

In some embodiments, the present disclosure provides a compound having the structure of Formula (I-1c1):

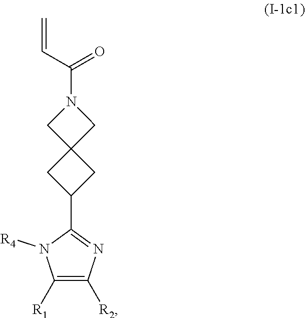

or a pharmaceutically acceptable salt, stereoisomer, or atropisomer thereof, wherein R₁, R₂, and R₄ are as defined herein.

In some embodiments, the compound of the present disclosure is a compound of Table A or a pharmaceutically acceptable salt thereof.

TABLE A

| | |
|---|---|
| 1 | 1-[6-[1-methyl-4-(1-methylindazol-5-yl)-5-(5-methyl- |
| | 1H-indazol-4-yl)imidazol-2-yl]-2- |
| | azaspiro[3.3]heptan-2-yl]prop-2-en-1-one |
| | 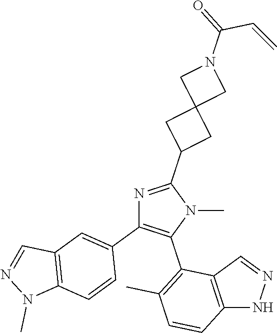 [SMILES: C=CC(=O)N1CC2(CC(c3nc(-c4ccc5c(cnn5C)c4)c(-c4c(C)ccc5[nH]ncc45)n3C)C2)C1] |
| 2 | 1-[6-[1-methyl-5-(5-methyl-1H-indazol-4-yl)-4- |
| | phenyl-imidazol-2-yl]-2-azaspiro[3.3]heptan-2- |
| | yl]prop-2-en-1-one |
| | 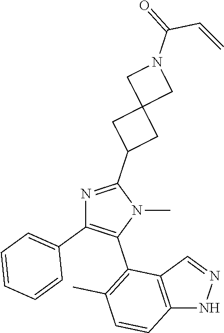 [SMILES: C=CC(=O)N1CC2(CC(c3nc(-c4ccccc4)c(-c4c(C)ccc5[nH]ncc45)n3C)C2)C1] |
| 3 | 1-[6-[1-methyl-5-(5-methyl-1H-indazol-4-yl)-4-(3- |
| | pyridyl)imidazol-2-yl]-2-azaspiro[3.3]heptan-2- |
| | yl]prop-2-en-1-one |
| | 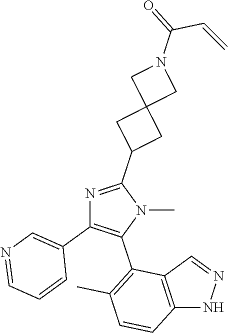 [SMILES: C=CC(=O)N1CC2(CC(c3nc(-c4cccnc4)c(-c4c(C)ccc5[nH]ncc45)n3C)C2)C1] |
| 4 | 1-[6-[4-(2-fluoro-4-methoxy-phenyl)-1-methyl-5-(5- |
| | methyl-1H-indazol-4-yl)imidazol-2-yl]-2- |
| | azaspiro[3.3]heptan-2-yl]prop-2-en-1-one |
| | 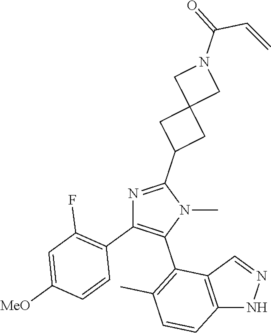 [SMILES: C=CC(=O)N1CC2(CC(c3nc(-c4ccc(OC)cc4F)c(-c4c(C)ccc5[nH]ncc45)n3C)C2)C1] |
| 5 | 1-[6-[1-methyl-5-(5-methyl-1H-indazol-4-yl)-4-(4- |
| | morpholinophenyl)imidazol-2-yl]-2- |
| | azaspiro[3.3]heptan-2-yl]prop-2-en-1-one |
| | 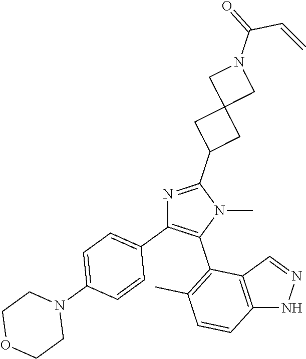 [SMILES: C=CC(=O)N1CC2(CC(c3nc(-c4ccc(N5CCOCC5)cc4)c(-c4c(C)ccc5[nH]ncc45)n3C)C2)C1] |
| 6 | 1-[6-[1-methyl-5-(5-methyl-1H-indazol-4-yl)-4-(3- |
| | morpholinophenyl)imidazol-2-yl]-2- |
| | azaspiro[3.3]heptan-2-yl]prop-2-en-1-one |
| | 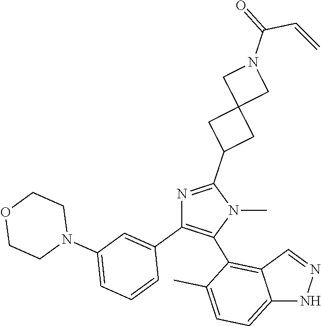 [SMILES: C=CC(=O)N1CC2(CC(c3nc(-c4cccc(N5CCOCC5)c4)c(-c4c(C)ccc5[nH]ncc45)n3C)C2)C1] |
| 7 | 1-[6-[1-methyl-4-(1-methylindazol-6-yl)-5-(5-methyl- |
| | 1H-indazol-4-yl)imidazol-2-yl]-2- |
| | azaspiro[3.3]heptan-2-yl]prop-2-en-1-one |
| | 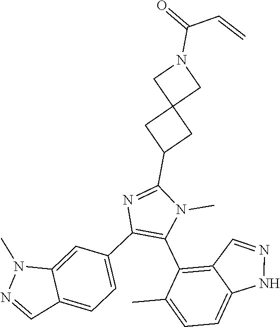 [SMILES: C=CC(=O)N1CC2(CC(c3nc(-c4ccc5cnn(C)c5c4)c(-c4c(C)ccc5[nH]ncc45)n3C)C2)C1] |
| 8 | 1-[6-[4-[4-(4-cyclopropylimidazol-1-yl)phenyl]-1- |
| | methyl-5-(5-methyl-1H-indazol-4-yl)imidazol-2-yl]-2- |
| | azaspiro[3.3]heptan-2-yl]prop-2-en-1-one |
| | 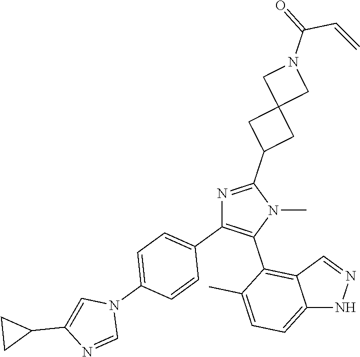 [SMILES: C=CC(=O)N1CC2(CC(c3nc(-c4ccc(-n5cnc(C6CC6)c5)cc4)c(-c4c(C)ccc5[nH]ncc45)n3C)C2)C1] |
| 9 | 1-[6-[4-(1,2-dimethylbenzimidazol-5-yl)-1-methyl-5- |
| | (5-methyl-1H-indazol-4-yl)imidazol-2-yl]-2- |
| | azaspiro[3.3]heptan-2-yl]prop-2-en-1-one |
| | 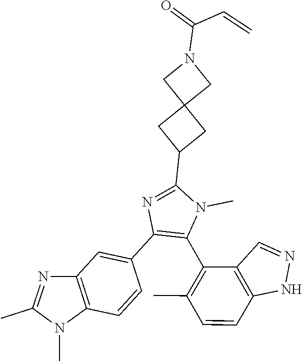 [SMILES: C=CC(=O)N1CC2(CC(c3nc(-c4ccc5c(c4)nc(C)n5C)c(-c4c(C)ccc5[nH]ncc45)n3C)C2)C1] |
| 10 | 1-[6-[1-methyl-5-(5-methyl-1H-indazol-4-yl)-4-[1-(2- |
| | morpholinoethyl)pyrazol-4-yl]imidazol-2-yl]-2- |
| | azaspiro[3.3]heptan-2-yl]prop-2-en-1-one |
| | 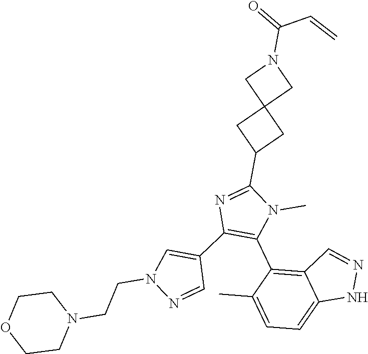 [SMILES: C=CC(=O)N1CC2(CC(c3nc(-c4cnn(CCN5CCOCC5)c4)c(-c4c(C)ccc5[nH]ncc45)n3C)C2)C1] |
| 11 | 1-[6-[4-(1,4-dimethylindazol-5-yl)-1-methyl-5-(5- |
| | methyl-1H-indazol-4-yl)imidazol-2-yl]-2- |
| | azaspiro[3.3]heptan-2-yl]prop-2-en-1-one |
| | 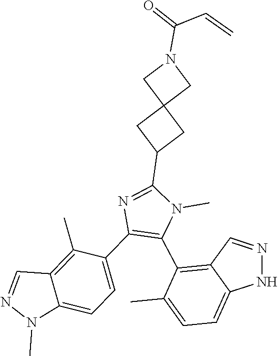 [SMILES: C=CC(=O)N1CC2(CC(c3nc(-c4ccc5c(cnn5C)c4C)c(-c4c(C)ccc5[nH]ncc45)n3C)C2)C1] |
| 12 | 1-[6-[4-(1,3-dimethylpyrazol-4-yl)-1-methyl-5-(5- |
| | methyl-1H-indazol-4-yl)imidazol-2-yl]-2- |
| | azaspiro[3.3]heptan-2-yl]prop-2-en-1-one |
| | 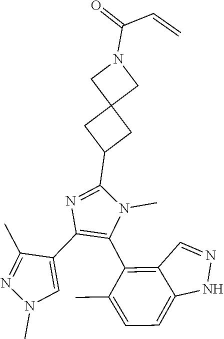 [SMILES: C=CC(=O)N1CC2(CC(c3nc(-c4cn(C)nc4C)c(-c4c(C)ccc5[nH]ncc45)n3C)C2)C1] |
| 13 | 1-[6-[4-(1-ethylpyrazol-4-yl)-1-methyl-5-(5-methyl- |
| | 1H-indazol-4-yl)imidazol-2-yl]-2- |
| | azaspiro[3.3]heptan-2-yl]prop-2-en-1-one |
| | 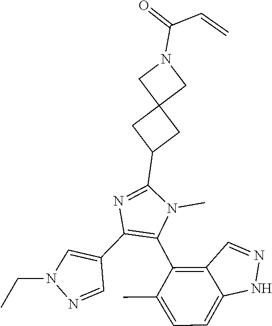 [SMILES: C=CC(=O)N1CC2(CC(c3nc(-c4cnn(CC)c4)c(-c4c(C)ccc5[nH]ncc45)n3C)C2)C1] |
| 14 | 1-[6-[1-methyl-4-(2-methylindazol-5-yl)-5-(5-methyl- |
| | 1H-indazol-4-yl)imidazol-2-yl]-2- |
| | azaspiro[3.3]heptan-2-yl]prop-2-en-1-one |
| | 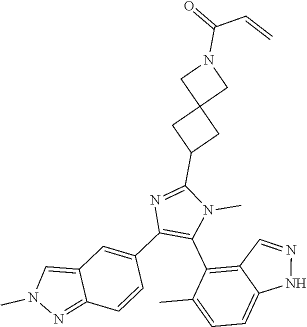 [SMILES: C=CC(=O)N1CC2(CC(c3nc(-c4ccc5nn(C)cc5c4)c(-c4c(C)ccc5[nH]ncc45)n3C)C2)C1] |
| 15 | 1-[6-[4-[1-(2-methoxyethyl)pyrazol-4-yl]-1-methyl-5- |
| | (5-methyl-1H-indazol-4-yl)imidazol-2-yl]-2- |
| | azaspiro[3.3]heptan-2-yl]prop-2-en-1-one |
| | 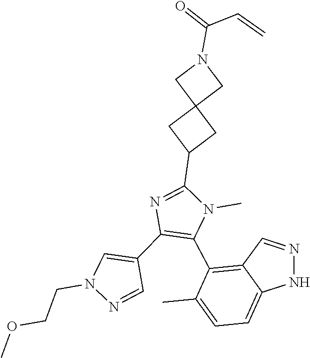 [SMILES: C=CC(=O)N1CC2(CC(c3nc(-c4cnn(CCOC)c4)c(-c4c(C)ccc5[nH]ncc45)n3C)C2)C1] |
| 16 | 1-[6-[4-(1,3-dimethylindazol-5-yl)-1-methyl-5-(5- |
| | methyl-1H-indazol-4-yl)imidazol-2-yl]-2- |
| | azaspiro[3.3]heptan-2-yl]prop-2-en-1-one |
| | 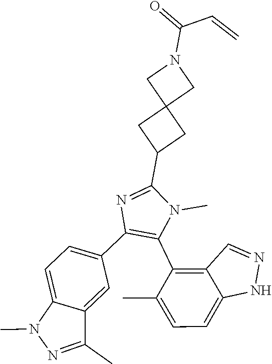 [SMILES: C=CC(=O)N1CC2(CC(c3nc(-c4ccc5c(c4)c(C)nn5C)c(-c4c(C)ccc5[nH]ncc45)n3C)C2)C1] |
| 17 | 1-[6-[4-(1,3-benzoxazol-5-yl)-1-methyl-5-(5-methyl- |
| | 1H-indazol-4-yl)imidazol-2-yl]-2- |
| | azaspiro[3.3]heptan-2-yl]prop-2-en-1-one |
| | 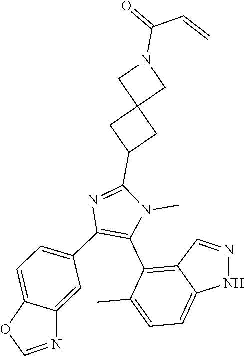 [SMILES: C=CC(=O)N1CC2(CC(c3nc(-c4ccc5ocnc5c4)c(-c4c(C)ccc5[nH]ncc45)n3C)C2)C1] |
| 18 | 1-[6-[1-methyl-4-(4-methyl-2,3-dihydro-1,4- |
| | benzoxazin-7-yl)-5-(5-methyl-1H-indazol-4- |
| | yl)imidazol-2-yl]-2-azaspiro[3.3]heptan-2-yl]prop-2- |
| | en-1-one |
| | 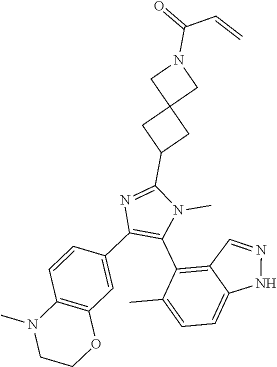 [SMILES: C=CC(=O)N1CC2(CC(c3nc(-c4ccc5c(c4)OCCN5C)c(-c4c(C)ccc5[nH]ncc45)n3C)C2)C1] |
| 19 | N,N-dimethyl-2-[4-[1-methyl-5-(5-methyl-1H-indazol- |
| | 4-yl)-2-(2-prop-2-enoyl-2-azaspiro[3.3]heptan-6- |
| | yl)imidazol-4-yl]phenoxy]acetamide |
| | 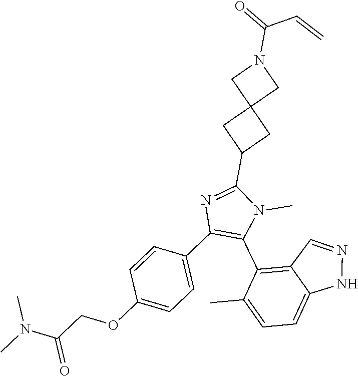 [SMILES: C=CC(=O)N1CC2(CC(c3nc(-c4ccc(OCC(=O)N(C)C)cc4)c(-c4c(C)ccc5[nH]ncc45)n3C)C2)C1] |
| 20 | 1-[6-[1-methyl-5-(5-methyl-1H-indazol-4-yl)-4-[4-(2- |
| | morpholino-2-oxo-ethoxy)phenyl]imidazol-2-yl]-2- |
| | azaspiro[3.3]heptan-2-yl]prop-2-en-1-one |
| | 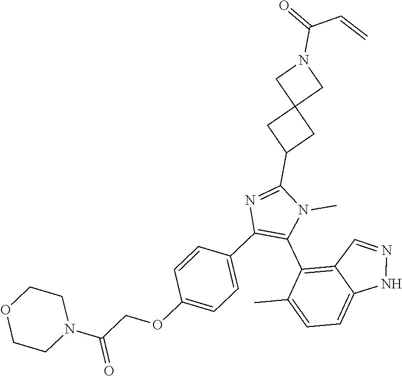 [SMILES: C=CC(=O)N1CC2(CC(c3nc(-c4ccc(OCC(=O)N5CCOCC5)cc4)c(-c4c(C)ccc5[nH]ncc45)n3C)C2)C1] |
| 21 | 1-[6-[1-methyl-5-(1-methylindazol-5-yl)-4-(5-methyl- |
| | 1H-indazol-4-yl)imidazol-2-yl]-2- |
| | azaspiro[3.3]heptan-2-yl]prop-2-en-1-one |
| | 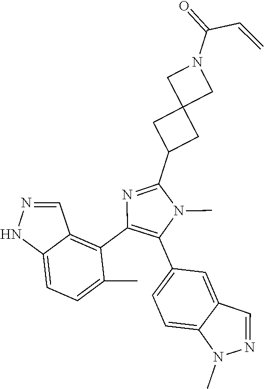 [SMILES: C=CC(=O)N1CC2(CC(c3nc(-c4c(C)ccc5[nH]ncc45)c(-c4ccc5c(cnn5C)c4)n3C)C2)C1] |
| 22 | 1-[6-[5-(3-hydroxy-1-naphthyl)-1-methyl-4-(1- |
| | methylindazol-5-yl)imidazol-2-yl]-2- |
| | azaspiro[3.3]heptan-2-yl]prop-2-en-1-one |
| | 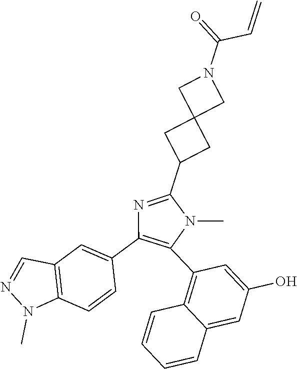 [SMILES: C=CC(=O)N1CC2(CC(c3nc(-c4ccc5c(cnn5C)c4)c(-c4cc(O)cc5ccccc45)n3C)C2)C1] |
| 23 | 1-[6-[5-(3-hydroxy-1-naphthyl)-1-methyl-4-(4- |
| | morpholinophenyl)imidazol-2-yl]-2- |
| | azaspiro[3.3]heptan-2-yl]prop-2-en-1-one |
| | 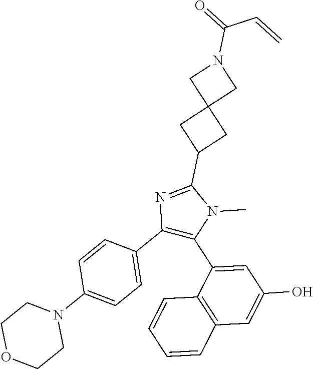 [SMILES: C=CC(=O)N1CC2(CC(c3nc(-c4ccc(N5CCOCC5)cc4)c(-c4cc(O)cc5ccccc45)n3C)C2)C1] |
| 24 | 1-[6-[5-(3-hydroxy-1-naphthyl)-1-methyl-4-(2- |
| | methylindazol-5-yl)imidazol-2-yl]-2- |
| | azaspiro[3.3]heptan-2-yl]prop-2-en-1-one |
| | 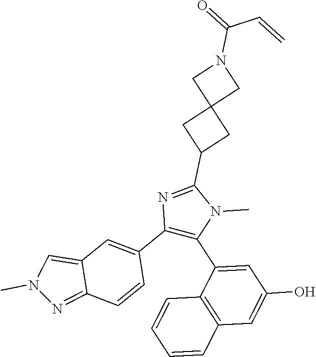 [SMILES: C=CC(=O)N1CC2(CC(c3nc(-c4ccc5nn(C)cc5c4)c(-c4cc(O)cc5ccccc45)n3C)C2)C1] |
| 25 | 1-[6-[5-(3-hydroxy-1-naphthyl)-1-methyl-4-[4-(2- |
| | morpholino-2-oxo-ethoxy)phenyl]imidazol-2-yl]-2- |
| | azaspiro[3.3]heptan-2-yl]prop-2-en-1-one |
| | 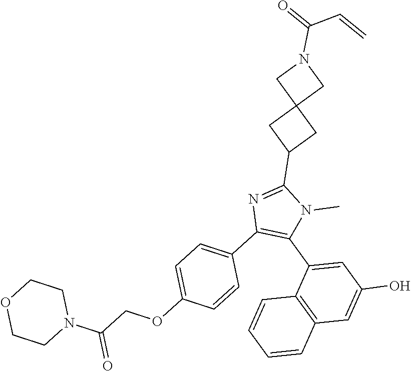 [SMILES: C=CC(=O)N1CC2(CC(c3nc(-c4ccc(OCC(=O)N5CCOCC5)cc4)c(-c4cc(O)cc5ccccc45)n3C)C2)C1] |

In some embodiments, the compound of the present disclosure is one of compounds 1-33 provided in Table 6, Table 9, or the Examples.

As described herein, the compounds of the present disclosure include pharmaceutically acceptable salts, solvates and/or stereoisomers thereof. Where a compound has a stereocentre, both (R) and (S) stereoisomers are contemplated by the invention, equally mixtures of stereoisomers or a racemic mixture are completed by the present application. Where a compound of the disclosure has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diastereomeric mixtures. Where the mixture is a mixture of enantiomers the enantiomeric excess may be any of those disclosed above. Where the compound is a single stereoisomer the compounds may still contain other diastereoisomers or enantiomers as impurities. Hence a single stereoisomer does not necessarily have an enantiomeric excess (e.e.) or diastereomeric excess (d.e.) of 100%, but could have an e.e. or d.e. of about at least 85%, at least 60% or less. For example, the e.e. or d.e. may be 95% or more, 90% or more, 80% or more, 70% or more, 60% or more, 50% or more, 40% or more, 30% or more, 20% or more, or 10% or more.

The present disclosure also contemplates pharmaceutically acceptable salts of the compounds described herein. These may include the acid addition and base salts of the compounds. These may be acid addition and base salts of the compounds. In addition, the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds disclosed herein (e.g., the compounds of formula (I)) may be prepared by one or more of three methods:
(i) by reacting the compound of the disclosure with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the disclosure may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the disclosure are complexes such as clathrates, drug-host inclusion complexes wherein, in contrast to solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of any formula include references to salts, solvates and complexes thereof and to solvates and complexes of salts thereof.

The compounds of the present disclosure include compounds of a number of formulae as herein defined, including all polymorphs and crystal habits thereof, prodrugs and isomers thereof (including optical, geometric and tautomeric isomers) as hereinafter defined and isotopically-labelled compounds of the invention.

The present disclosure also includes all pharmaceutically acceptable isotopically-labelled compounds of the invention wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Before purification, the compounds of the present disclosure may exist as a mixture of enantiomers depending on the synthetic procedure used. The enantiomers can be separated by conventional techniques known in the art. Thus, the present disclosure covers individual enantiomers as well as mixtures thereof.

For some of the steps of the process of preparation of the compounds of the disclosure, it may be necessary to protect potential reactive functions that are not wished to react, and to cleave said protecting groups in consequence. In such a case, any compatible protecting radical can be used. In particular methods of protection and deprotection such as those described by T.W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present disclosure as well as intermediates for the preparation thereof can be purified according to various well-known methods, such as for example crystallization or chromatography.

### Pharmaceutical Compositions

In some embodiments, the present disclosure also provides pharmaceutical compositions comprising one or more compounds disclosed herein (e.g., a compound of Formula (I-1), Formula (I-2), Formula (I-1a), Formula (I-1a1), Formula (I-1b), Formula (I-1 b1), Formula (I-1b2), Formula (I-1c), Formula (I-1c1), or Table A), or a pharmaceutically acceptable solvate, hydrate, tautomer, or salt thereof; and a pharmaceutically acceptable excipient or adjuvant.

In some embodiments, a pharmaceutical composition comprising one or more compounds disclosed herein, or a pharmaceutically acceptable solvate, hydrate, tautomer, or salt thereof, further comprises a pharmaceutically acceptable carrier. In some embodiments, a pharmaceutically acceptable carrier includes a pharmaceutically acceptable excipient, binder, and/or diluent. In some embodiments, suitable pharmaceutically acceptable excipients include, but are not limited to, acacia, animal oils, benzyl alcohol, benzyl benzoate, calcium stearate, carbomers, cetostearyl alcohol, cetyl alcohol, cholesterol, cyclodextrins, dextrose, diethanolamine, emulsifying wax, ethylene glycol palmitostearate, glycerin, glycerin monostearate, glycerol stearate, glyceryl monooleate, glyceryl monostearate, hydrous, histidine, hydrochloric acid, hydroxpropyl cellulose, hydroxypropyl-β-cyclodextrin (HPBCD), hypromellose (hydroxypropyl methylcellulose (HPMC)), lanolin, lanolin alcohols, lecithin, medium-chain triglycerides, metallic soaps, methylcellulose, mineral oil, monobasic sodium phosphate, monoethanolamine, oleic acid, polyethylene glycols (PEG 3350, PEG 4000, PEG 6000), polyoxyethylene-polyoxypropylene copolymer (poloxamer), polyoxyethylene alkyl ethers, polyoxyethylene castor oil, polyoxyethylene castor oil derivatives, polyoxyethylene sorbitan fatty acid esters, polyoxyethylene stearates, polysorbate, polyoxyethylene (20) sorbitan monolaurate (Tween 20, Polysorbate 20), polyoxyethylene (20) sorbitan monooleate (Tween 80, Polysorbate 80), povidone, propylene glycol alginate, saline, sodium chloride, sodium citrate, sodium citrate dihydrate, sodium hydroxide, sodium lauryl sulfate, sodium phosphate monobasic, sodium phosphate dibasic, sorbitan esters, stearic acid, stearyl alcohol, sunflower oil, tragacanth, triethanolamine, vegetable oils, water, xanthan gum, or a combinations thereof.

In some embodiments, the pharmaceutical composition further comprises an additional pharmaceutically active agent. In some embodiments, the additional pharmaceutically active is an anti-inflammatory agent, an anti-fibrotic agent, a chemotherapeutic, an anti-cancer agent, an immunosuppressant, an anti-tumour vaccine, a cytokine therapy, or a tyrosine kinase inhibitor. Non-limiting examples of additional active active agents suitable for use in a pharmaceutical composition disclosed herein are provided below.

### Methods of Treatment

In some embodiments, the present disclosure provides a method for treating a condition modulated by RAS proteins in a subject in need thereof, the method comprising, administering to the subject a therapeutically effect amount of a compound disclosed herein (e.g., a compound of Formula (I-1), Formula (I-2), Formula (I-1a), Formula (I-1a1), Formula (I-1b), Formula (I-1b1), Formula (I-1b2), Formula (I-1c), Formula (I-1c1), or Table A), or a pharmaceutically acceptable salt thereof.

In some embodiments, the present disclosure provides a compound disclosed herein for use in treating a condition modulated by RAS proteins in a subject in need thereof.

In some embodiments, the present disclosure provides the use of a compound disclosed herein for the manufacture of a medicament for treating a condition modulated by RAS proteins in a subject in need thereof.

In some embodiments, the compound of the present disclosure is an inhibitor of KRAS proteins.

In some embodiments, the present disclosure provides a method for treating a condition modulated by RAS proteins in a subject in need thereof, the method comprising, administering to the subject a pharmaceutical composition disclosed herein.

In some embodiments, the condition modulated by RAS proteins is cancer. In some embodiments, the cancer is selected from the group consisting of sarcoma, melanoma, skin cancer, haematological tumors, lymphoma, carcinoma, and leukemia.

As discussed above, RAS signalling is instrumental in a number of conditions. In some embodiments, the condition is treatable by inhibiting RAS. In some embodiments, the condition treatable by the inhibition of RAS is cancer. In some embodiments, the cancer is sarcoma, melanoma, skin cancer, haematological tumors, lymphoma, carcinoma, or leukemia. In some embodiments, the cancer is cervical cancer, multiple myeloma, stomach cancer, bladder cancer, uterine cancer, esophageal squamous cell carcinoma, gastric cancer, glioblastomas, astrocytomas; retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, head and neck squamous cell carcinoma or cancer of the head and neck. In some embodiments, the condition is lung cancer, esophageal cancer, colorectal cancer, stomach cancer, bladder cancer, hepatocellular cancer, uterine cancer, cervical cancer, pancreatic cancer or ovarian cancer.

In some embodiments, the present disclosure provides a compound having a RAS binding affinity within category A, B or C as defined below. In some embodiments, the present disclosure provides compounds having a RAS G12C binding such that ≥70% of the compound remains at the indicated timepoint (category A). In some embodiments, is the present disclosure provides compounds having a RAS G12C binding such that 30%-70% of the compound remains at the indicated timepoint (category B). In some embodiments, the present disclosure provides compounds having a RAS G12C binding such that ≤30% of the compound remains at the indicated timepoint (category C). Optionally, the binding activity is determined using the assay for RAS inhibition defined in the examples.

In some embodiments, one or more compounds of the present disclosure is combined with one or more additional pharmaceutical agents, for example anti-inflammatory agents, anti-fibrotic agents, chemotherapeutics, anti-cancer agents, immunosuppressants, anti-tumour vaccines, cytokine therapy, or tyrosine kinase inhibitors, for the treatment of conditions modulated by the inhibition of RAS proteins, for example cancer, sarcoma, melanoma, skin cancer, haematological tumors, lymphoma, carcinoma, and leukemia.

In some embodiments, the present disclosure provides compounds for use as a medicament.

In some embodiments, the present disclosure provides a method of treating a condition modulated by inhibition of KRAS proteins in a subject in need thereof, the method comprising administering a therapeutically effective amount of a compound disclosed herein (e.g., a compound of Formula (I-1), Formula (I-2), Formula (I-1a), Formula (I-1a1), Formula (I-1b), Formula (I-1b1), Formula (I-1b2), Formula (I-1c), Formula (I-1c1), or Table A) or a pharmaceutical composition to the subject.

In some embodiments, the present disclosure provides a pharmaceutical formulation comprising a compound disclosed herein and a pharmaceutically acceptable excipient for use in treating a condition modulated by inhibition of KRAS proteins in a subject in need thereof.

In some embodiments, the present disclosure provides a compound for use in the treatment of a condition which is modulated by RAS. In some embodiments, conditions that are modulated by RAS are conditions that are treatable by the inhibition of RAS using a compound of the present disclosure. A compound of any formula disclosed herein may be for use in the treatment of a condition treatable by the inhibition of RAS.

In some embodiments, the condition treatable by a compound disclosed herein is associated with a KRAS mutation. In some embodiments, the KRAS mutation is a G12C mutation.

The method of treatment or the compound for use in the treatment of cancer, sarcoma, melanoma, skin cancer, haematological tumors, lymphoma, carcinoma, and leukemia as defined herein may be applied as a sole therapy or be a combination therapy with an additional active agent.

In some embodiments, the method of treatment or the compound for use in the treatment of cancer, sarcoma, melanoma, skin cancer, haematological tumors, lymphoma, carcinoma, and leukemia comprises, in addition to the compound of disclosed herein (e.g., a compound of formula (I)), comprises one or more additional active agents. The additional active agents may be one or more active agents used to treat the condition being treated by the compound of the disclosure. In some embodiments, the additional active agent is one or more of the following active agents:
(i) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(ii) TNF inhibitors for example etanercept; monoclonal antibodies (e.g. infliximab (Remicade), adalimumab (Humira), certolizumab pegol (Cimzia), golimumab (Simponi)); fusion proteins (e.g. etanercept (Enbrel)); and 5-HT2A agonists (e.g. 2,5-dimethoxy-4-iodoamphetamine, TCB-2, lysergic acid diethylamide (LSD), lysergic acid dimethylazetidide);
(iii) anti-inflammatory drugs, for example non-steroidal anti-inflammatory drugs;
(iv) dihydrofolate reductase inhibitors/antifolates, for example methotrexate, trimethoprim, brodimoprim, tetroxoprim, iclaprim, pemetrexed, ralitrexed and pralatrexate; and
(v) immunosuppressants for example cyclosporins, tacrolimus, sirolimus pimecrolimus, angiotensin II inhibitors (e.g. Valsartan, Telmisartan, Losartan, Irbesatan, Azilsartan, Olmesartan, Candesartan, Eprosartan) and ACE inhibitors e.g. sulfhydryl-containing agents (e.g. Captopril, Zofenopril), dicarboxylate-containing agents (e.g. Enalapril, Ramipril, Quinapril, Perindopril, Lisinopril, Benazepril, Imidapril, Zofenopril, Trandolapril), phosphate-containing agents (e.g. Fosinopril), casokinins, lactokinins and lactotripeptides.
(vi) anti-fibrotic agents for example: Pirfenidone, Nintedanib, Anti-IL-13 monoclonal antibodies (e.g. Tralokinumab, QAX576, Lebrikizumab), simtuzumab, FG-3019, lysophosphatidic acid receptor antagonists (e.g. BMS-986020, AM966), LOXL2 inhibitors, BET bromodomain inhibitors (e.g. JQ1), HDAC inhibitors (e.g. Vorinostat), thrombin inhibitors (e.g. Dabigatran), FactorXa inhibitors (e.g. Apixban, Rivaroxaban) 15PGDH inhibitors, anti-αvβ6 monoclonal antibodies (e.g. BG00011), Anti-CTGF monoclonal antibodies (e.g. FG-3019), PAR1 inhibitors, Nox4 inhibitors and PAI-1 inhibitors.
(vii) CNS therapies, for example: Levodopa, Dopamine agonists, Apomorphine, Glutamate antagonist, Anticholinergics, COMT inhibitors, MAO-B inhibitors, riluzole (Rilutek), Tetrabenazine (Xenazine), haloperidol (Haldol), chlorpromazine, risperidone (Risperdal), quetiapine (Seroquel), amantadine, levetiracetam (Keppra), clonazepam (Klonopin), Donepezil (Aricept), Galantamine (Razadyne), Rivastigmine (Exelon)), Memantine (Ebixa, Axura), Aducanumab, Ocrelizumab, interferon beta-1a (Avonex, Rebif), peginterferon beta-1a (Plegridy), teriflunomide (Aubagio), fingolimod (Gilenya), mitoxantrone (Novantrone), dimethyl fumarate (Tecfidera), natalizumab (Tysabri)

The method of treatment or the compound for use in the treatment of cancer, such as sarcoma, melanoma, skin cancer, haematological tumors, lymphoma, carcinoma, leukemia, and central nervous system disorders may involve, in addition to administering a compound disclosed herein or pharmaceutical composition thereof, conventional surgery or radiotherapy or chemotherapy. Such chemotherapy may include one or more of the following categories of anti-tumor agents:
(i) antiproliferative/antineoplastic drugs and combinations thereof, such as alkylating agents (for example cis platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophosphoramine, carmustine, lomustine, streptozocin and dacarbazine); antimetabolites (for example gemcitabine and antifolates such as fluoropyrimidines like 5 fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-a), etoposide, and teniposide;
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti erbB2 antibody trastuzumab [Herceptin™], the anti-EGFR antibody panitumumab, the anti erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 modulator;
(v) antiangiogenic agents such as those which inhibit the effects of vascular endothelial growth factor, for example the anti vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); and toll-like receptor modulators for example TLR-7 or TLR-9 agonists; and
(viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example PI3Kd inhibitors, for example idelalisib and perifosine; PD-1, PD-L1, PD-L2 and CTL4-A modulators, antibodies and vaccines; other IDO inhibitors (such as indoximod); anti-PD-1 monoclonal antibodies (such as MK-3475 and nivolumab); anti-PD-L1 monoclonal antibodies (such as MEDI-4736 and RG-7446); anti-PD-L2 monoclonal antibodies; and anti-CTLA-4 antibodies (such as ipilimumab);
(xii) chimeric antigen receptors, anticancer vaccines and arginase inhibitors.

Such combination treatment may be achieved by way of the simultaneous, sequential or separate dosing of the individual components of the treatment. Such combination products employ the compounds of the present disclosure within a therapeutically effective dosage range described herein and the additional pharmaceutically-active agent within its approved dosage range.

Compounds of the disclosure may exist in a single crystal form or in a mixture of crystal forms or they may be amorphous. Thus, compounds of the disclosure intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

### Administration

For the above-mentioned compounds of the present disclosure, the dosage administered will vary with the compound employed, the mode of administration, the treatment desired and the disorder indicated. For example, if the compound of the disclosure is administered orally, then the daily dosage of the compound of the disclosure may be in the range from 0.01 micrograms per kilogram body weight (μg/kg) to 100 milligrams per kilogram body weight (mg/kg).

A compound of the present disclosure (e.g., a compound of Formula (I-1), Formula (I-2), Formula (I-1a), Formula (I-1a1), Formula (I-1b), Formula (I-1b1), Formula (I-1b2), Formula (I-1c), Formula (I-1c1), or Table A), or pharmaceutically acceptable salt thereof, may be used on its own, but in some embodiments is administered in the form of a pharmaceutical composition in which the compounds of the disclosure, or pharmaceutically acceptable salt thereof, is provided with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds disclosed herein, the pharmaceutical composition, which is used to administer the compound, comprises from 0.05 to 99% by weight of compound, from 0.05 to 80% by weight of compound, from 0.10 to 70% by weight of compound, or from 0.10 to 50% by weight, with all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration, the compounds of the disclosure may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the disclosure may be admixed with, for example, a vegetable oil or polyethylene glycol. Hard gelatine capsules may contain granules of the compound using either the above-mentioned excipients for tablets. Also, liquid or semisolid formulations of the compound of the disclosure may be filled into hard gelatine capsules. Liquid preparations for oral application may be in the form of syrups or suspensions, for example, solutions containing the compound of the disclosure, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration, the compounds of the disclosure may be administered as a sterile aqueous or oily solution.

The size of the dose for therapeutic purposes of compounds of the disclosure will naturally vary according to the nature and severity of the conditions, the age and sex of the animal or patient and the route of administration, according to well-known principles of medicine.

Dosage levels, dose frequency, and treatment durations of compounds of the disclosure are expected to differ depending on the formulation and clinical indication, age, and co-morbid medical conditions of the patient.

### EXAMPLES

As used herein the following terms have the meanings given “Cbz” refers to carboxybenzyl; “DCM” refers to dichloromethane; “DIPEA” refers to N,N-Diisopropylethylamine; “DMF” refers to N,N-dimethylformamide; “DHP” refers to 3,4-Dihydro-2H-pyran; “dppf” refers to 1,1′-Bis(diphenylphosphino)ferrocene; “dtbpf” refers to 1,1′-Bis(di-tert-butylphosphino)ferrocene; “EtOAc” refers to ethyl acetate; “LCMS” refers to liquid chromatography/mass spectrometry; “MeCN” refers to acetonitrile; “min” refers to minutes; “r.t.” refers to room temperature; “T3P” refers to propylphosphonic anhydride; “THF” refers to tetrahydrofuran; “THP” refers to tetrahydropyran.

Solvents, reagents and starting materials were purchased from commercial vendors and used as received unless otherwise described. All reactions were performed at room temperature unless otherwise stated. Compound identity and purity confirmations were performed by LCMS UV using a Waters Acquity SQ Detector 2. The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in MeCN) according to the gradients outlined in Table 1 below. Retention times RT are reported in minutes.

TABLE 1

| | | |
|---|---|---|
| Time (min) | % A | % B |

Method 1

| | | |
|---|---|---|
| 0 | 95 | 5 |
| 0.3 | 95 | 5 |
| 2 | 5 | 95 |
| 2.6 | 95 | 5 |
| 3 | 95 | 5 |

Method 2

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Compound identity and purity confirmations were also performed by LCMS UV using a Waters Acquity H-Class QDA Detector. The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in MeCN) according to the gradients outlined in Table 2 below. Retention times RT are reported in minutes.

TABLE 2
Method 3

| Time (min) | % A | % B |
|---|---|---|
| 0 | 95 | 5 |
| 0.3 | 95 | 5 |
| 2 | 5 | 95 |
| 2.6 | 95 | 5 |
| 3 | 95 | 5 |

NMR was also used to characterise final compounds. NMR spectra were obtained on a Bruker AVIII 400 Nanobay with 5 mm BBFO probe. Optionally, compound Rf values on silica thin layer chromatography (TLC) plates were measured.

Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector. Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in MeCN) according to the gradient outlined in Table 3 below.

TABLE 3

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

Chemical names in this document were generated using mol2nam—Structure to Name Conversion by OpenEye Scientific Software. Starting materials were purchased from commercial sources or synthesised according to literature procedures.

### Synthesis of Intermediates

### Intermediate 1-tert-butyl-6-(4,5-dibromo-1-methyl-imidazol-2-yl)-2-azaspiro[3.3]heptane-2-carboxylate

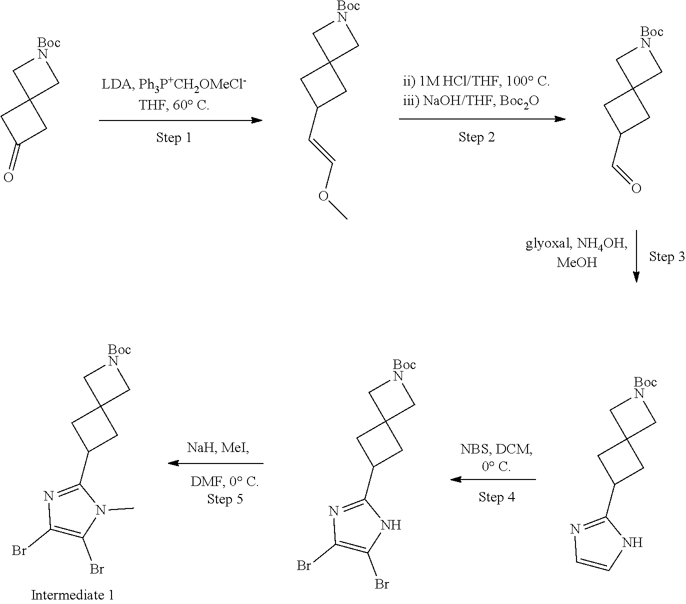 [SMILES: CC(C)(C)OC(=O)N1CC2(CC(=O)C2)C1 ; CO/C=C/C1CC2(C1)CN(C(=O)OC(C)(C)C)C2 ; CC(C)(C)OC(=O)N1CC2(CC(C=O)C2)C1 ; Cn1c(C2CC3(C2)CN(C(=O)OC(C)(C)C)C3)nc(Br)c1Br ; CC(C)(C)OC(=O)N1CC2(CC(c3nc(Br)c(Br)[nH]3)C2)C1 ; CC(C)(C)OC(=O)N1CC2(CC(c3ncc[nH]3)C2)C1]

### Step 1—tert-butyl 6-(methoxymethylene)-2-azaspiro[3.3]heptane-2-carboxylate

n-Butyllithium solution (59.64 mL, 149.11 mmol) was slowly added to a stirred solution of DIPEA (20.9 mL, 149.11 mmol) and THF (100 mL) at −78° C. under a nitrogen atmosphere. The mixture was stirred at −78° C. for 30 minutes and then allowed to warm to 0° C. A stirred mixture of methoxymethyltriphenylphosphoniumchloride (48.68 g, 142.01 mmol) in THF (250 mL) at 0° C. was prepared and the LDA mixture was added slowly via cannula to this mixture. The resulting mixture was allowed to warm to room temperature and stir for 30 minutes. A solution of tert-butyl 6-oxo-2-azaspiro[3.3]heptane-2-carboxylate (15 g, 71 mmol) in THE (100 mL) was then slowly added and the resulting mixture heated to 60° C. for 2 hours and then cooled to room temperature and solvent removed in vacuo. The residue was partitioned between water (300 mL) and EtOAc (300 mL). The aqueous layer was extracted with EtOAc (200 mL) and the combined organic layers were dried over Na₂SO₄ and solvent removed in vacuo. The reside was purified by column chromatography using an eluent of 0-50% EtOAc in petroleum ether to give tert-butyl 6-(methoxymethylene)-2-azaspiro[3.3]heptane-2-carboxylate (16.99 g, 71 mmol, 100% yield) as a brown oil which was used directly in the next step.

UPLC-MS (ES+, Method 1): 1.87 min, m/z 240.1 [M+H]⁺

### Step 2—tert-butyl-6-formyl-2-azaspiro[3.3]heptane-2-carboxylate

tert-butyl 6-(methoxymethylene)-2-azaspiro[3.3]heptane-2-carboxylate (16.99 g, 71. mmol) was stirred in HCl (1M) (180.2 mL, 180.2 mmol) and THE (70 mL) at 100° C. for 2 hours. The reaction was then cooled to room temperature and extracted with EtOAc (2×200 mL) to remove remaining Wittig reagent from the previous step. The aqueous layer was then taken to ˜pH 9 with 5M NaOH and Sodium hydroxide solid (2.43 g, 60.67 mmol) was then added along with Boc₂O (23.24 g, 106.49 mmol) and the resulting mixture stirred for 3 hours. The reaction mixture was then diluted with water (200 mL) and the resulting mixture was extracted with EtOAc (2×300 mL). The combined organic layers were dried over Na₂SO₄ and solvent removed in vacuo to give tert-butyl 6-formyl-2-azaspiro[3.3]heptane-2-carboxylate (15.9 g, 71 mmol, 100% yield) as a light yellow oil which was used in the next step without further purification.

UPLC-MS (ES+, Method 1): 1.55 min, m/z 248.1 [M+Na]⁺; 170.0 [M-tBu+H]⁺

¹H-NMR (400 MHz, CDCl₃) δ/ppm: 9.66 (1H, s), 3.88-3.76 (4H, m), 3.09-2.97 (1H, m), 2.42-2.59 (4H, m), 1.36 (9H, s).

### Step 3—tert-butyl 6-(1H-imidazol-2-yl)-2-azaspiro[3.3]heptane-2-carboxylate

NH₄OH (137 mL, 3529 mmol) was added to a stirred solution of tert-butyl 6-formyl-2-azaspiro[3.3]heptane-2-carboxylate (15.9 g, 70.58 mmol) and glyoxal (19.35 mL, 423.47 mmol) at room temperature. After 1 hour the reaction mixture was concentrated in vacuo and the residue partitioned between water (200 mL) and EtOAc (200 mL). The organic layer was separated and the aqueous extracted with EtOAc (100 mL). The combined organic layers were dried over Na₂SO₄ and solvent removed in vacuo. The residue was purified by column chromatography using an eluent of 0-10% MeOH in DCM to give tert-butyl 6-(1H-imidazol-2-yl)-2-azaspiro[3.3]heptane-2-carboxylate (7.20 g, 27.34 mmol, 39% yield) as a yellow oil.

UPLC-MS (ES+, Method 1): 1.14 min, m/z 264.3 [M+H]⁺; 208.0 [M-tBu+H]⁺

¹H-NMR (400 MHz, DMSO-d₆) δ/ppm: 7.65 (1H, s), 7.02 (1H, s), 6.86 (1H, br s), 3.93 (2H, s), 3.77 (2H, s), 3.39-3.29 (1H, m), 2.47-2.31 (4H, m), 1.36 (9H, s).

### Step 4—tert-butyl 6-(4,5-dibromo-1H-imidazol-2-yl)-2-azaspiro[3.3]heptane-2-carboxylate

NBS (7.30 g, 41.01 mmol) was added to a stirred solution of tert-butyl 6-(1H-imidazol-2-yl)-2-azaspiro[3.3]heptane-2-carboxylate (7.20 g, 27.34 mmol) and DCM (200 mL) with ice cooling. After 5 minutes the reaction was poured into sat. aq. NaHCO₃ (200 mL) and DCM (200 mL) added. The organic layer was separated, washed with brine (200 mL), dried over Na₂SO₄ and solvent removed in vacuo to give tert-butyl 6-(4,5-dibromo-1H-imidazol-2-yl)-2-azaspiro[3.3]heptane-2-carboxylate (11.51 g, 27.34 mmol, 100% yield) as a yellow solid which was used in the next step without further purification.

UPLC-MS (ES+, Method 1): 1.75 min, m/z 363.9, 365.9, 367.9 [M-tBu+H]⁺

¹H-NMR (400 MHz, CDCl₃) δ/ppm: 4.02 (2H, s), 3.86 (2H, s), 3.41-3.31 (1H, m), 2.61-2.49 (4H, m), 1.44 (9H, s), exchangeable NH not seen.

### Step 5—tert-butyl 6-(4,5-dibromo-1-methyl-imidazol-2-yl)-2-azaspiro[3.3]heptane-2-carboxylate

NaH (60% dispersed in mineral oil) (1.13 g, 28.26 mmol) was added in 4 equal portions to a stirred solution of tert-butyl 6-(4,5-dibromo-1H-imidazol-2-yl)-2-azaspiro[3.3]heptane-2-carboxylate (11.9 g, 28.26 mmol) and DMF (100 mL) with ice cooling. The ice bath was then taken away and after 20 minutes Iodomethane (2.11 mL, 33.91 mmol) was added in one portion and stirring continued for 5 minutes after which time the reaction was complete by LCMS. Water (10 mL) was carefully added to quench the NaH and then DMF removed in vacuo. The residue was then partitioned between water (200 mL) and EtOAc (200 mL). The organic layer was separated and the aqueous extracted with EtOAc (200 mL). The combined organic layers were dried over Na₂SO₄ and solvent removed in vacuo. The residue was purified by column chromatography using an eluent of 0-5% MeOH in DCM to give tert-butyl 6-(4,5-dibromo-1-methyl-imidazol-2-yl)-2-azaspiro[3.3]heptane-2-carboxylate (5.2 g, 11.95 mmol, 42% yield) as a yellow solid.

UPLC-MS (ES+, Method 1): 1.75 min, m/z 433.9, 435.9, 437.9 [M+H]⁺; 377.9, 379.9, 381.9 [M-tBu+H]⁺

¹H-NMR (400 MHz, CDCl₃) δ/ppm: 4.03 (2H, s), 3.85 (2H, s), 3.47 (3H, s), 3.42-3.32 (1H, m), 2.66-2.49 (4H, m), 1.44 (9H, s).

### Intermediate 2—5-methyl-1-tetrahydropyran-2-yl-4-(4,4,5,5-tetramethyl-1,3,2-dioxaborolan-2-yl)indazole

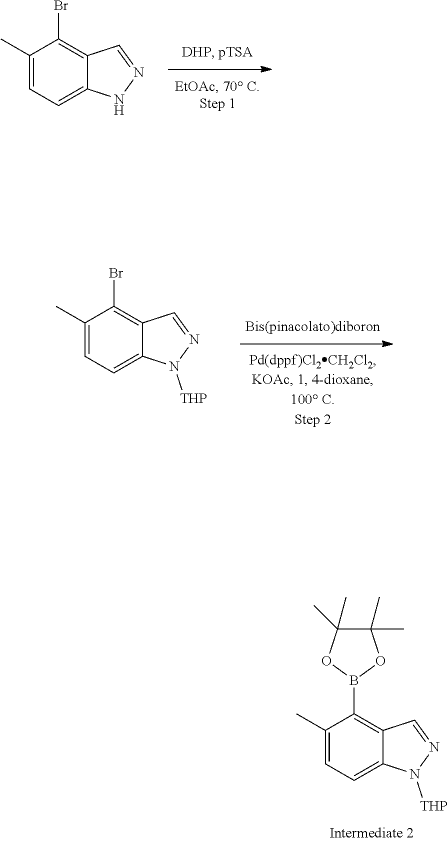

### Step 1—4-bromo-5-methyl-1-tetrahydropyran-2-yl-indazole

DHP (4.32 mL, 47.38 mmol) was added to a stirred solution of 4-bromo-5-methyl-1H-indazole (5.0 g, 23.69 mmol) and p-toluenesulfonic acid monohydrate (225 mg, 1.18 mmol) in EtOAc (200 mL) and the mixture was heated at 70° C. for 3 hours. The mixture was then cooled to room temperature and poured into sat. aq. Na₂CO₃ (200 mL) and the phases were separated. The aqueous phase was extracted with EtOAc (200 mL) and the combined organic layers were dried over sodium sulphate, filtered and concentrated in vacuo. The residue was purified by column chromatography, using an eluent of 0-50% EtOAc in petroleum ether to give 4-bromo-5-methyl-1-tetrahydropyran-2-yl-indazole (6.3 g, 21.45 mmol, 91% yield) as a white solid.

UPLC-MS (ES+, Method 1), 2.05 min, m/z 294.7, 296.7 [M+H]⁺

¹H-NMR (400 MHz, CDCl₃) δ/ppm: 7.98 (1H, d, J=5.2 Hz), 7.44 (1H, d, J=8.8 Hz), 7.24 (1H, d, J=8.8 Hz), 5.68 (1H, dd, J=10.2 Hz, 2.8 Hz), 4.03-3.97 (1H, m), 3.77-3.70 (1H, m), 2.60-2.51 (1H, m), 2.49 (3H, s), 2.21-2.03 (2H, m), 1.80-1.64 (3H, m).

### Step 2—5-methyl-1-tetrahydropyran-2-yl-4-(4,4,5,5-tetramethyl-1,3,2-dioxaborolan-2-yl)indazole

Bis(pinacolato)diboron (13.29 mL, 56.51 mmol), 4-bromo-5-methyl-1-tetrahydropyran-2-yl-indazole (13.9 g, 47.09 mmol), KOAc (13.8 g, 141.28 mmol), PdCl₂(dtbpf) (3.85 g, 4.71 mmol), and 1,4-dioxane (30 mL) were combined and stirred at room temperature. The reaction was degassed and flushed with nitrogen 3 times before being stirred at 100° C. under a nitrogen atmosphere for 18 hours. The reaction was then cooled to room temperature and solvent removed in vacuo. The residue was taken up in DCM (100 mL) and filtered through celite and the filter cake washed with DCM (100 mL). The filtrate was concentrated in vacuo to give 5-methyl-1-tetrahydropyran-2-yl-4-(4,4,5,5-tetramethyl-1,3,2-dioxaborolan-2-yl)indazole (9.6 g, 28.05 mmol, 60% yield) as a brown solid which was used in the next step without further purification.

UPLC-MS (ES+, Method 1): 2.14 min, m/z 343.3 [M+H]⁺.

¹H-NMR (400 MHz, CDCl₃) δ/ppm: 8.37 (1H, d, J=4.8 Hz), 7.54 (1H, d, J=8.8 Hz), 7.21 (1H, d, J=8.8 Hz), 5.68 (1H, dd, J=8.8 Hz, 2.8 Hz), 4.02-3.94 (1H, m), 3.76-3.67 (1H, m), 2.64 (3H, s), 2.61-2.50 (1H, m), 2-20-2.12 (1H, m), 2.06-1.98 (1H, m), 1.80-1.61 (3H, m), 1.39 (12H, s).

### Intermediate 3—1,4-dimethyl-5-(4,4,5,5-tetramethyl-1,3,2-dioxaborolan-2-yl)indazole

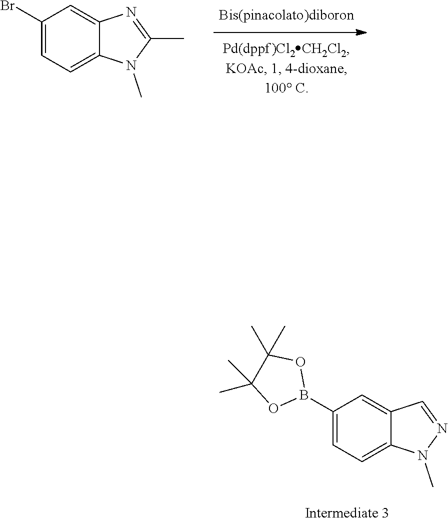 [SMILES: Cc1nc2cc(Br)ccc2n1C ; Cn1ncc2cc(B3OC(C)(C)C(C)(C)O3)ccc21]

Bis(pinacolato)diboron (1353.81 mg, 5.33 mmol), Pd(dppf)Cl₂·CH₂Cl₂ (363 mg, 0.44 mmol), KOAc (0.87 g, 8.89 mmol), 5-bromo-1,4-dimethyl-1H-indazole (1.0 g, 4.44 mmol) in 1,4-dioxane (11.87 mL) is stirred at 90° C. for 8 hours. Then, crude was cooled down at room temperature, filtered through a celite pad, solvent was evaporated and crude was purified using automated column chromatography to give 1,4-dimethyl-5-(4,4,5,5-tetramethyl-1,3,2-dioxaborolan-2-yl)indazole (1 g, 4.59 mmol, 84% yield) as a brown powder.

UPLC-MS (ES−, Method 3): 2.09 min, m/z 273.1 [M−H]⁻

¹H-NMR (400 MHz, DMSO-d₆) δ/ppm: 8.17 (1H, dd, J=7.0 Hz, 1.0 Hz), 7.63 (1H, d, J=8.5 Hz), 7.42-7.36 (1H, m), 4.02 (3H, s), 2.75 (3H, s), 1.32 (12H, s).

### Intermediate 4—1,2-dimethyl-5-(4,4,5,5-tetramethyl-1,3,2-dioxaborolan-2-yl)benzimidazole

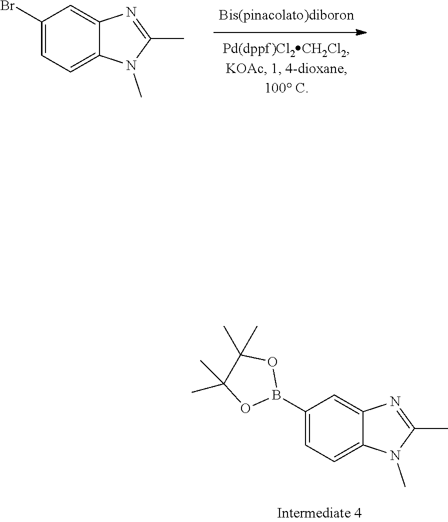

### 1,2-dimethyl-5-(4,4,5,5-tetramethyl-1,3,2-dioxaborolan-2-yl)benzimidazole

5-Bromo-1,2-dimethyl-1H-benzo[d]imidazole (1 g, 4.44 mmol), bis(pinacolato)diboron (1.35 g, 5.33 mmol), Pd(dppf)Cl₂·CH₂Cl₂ (363 mg, 0.44 mmol), KOAc (0.87 g, 8.89 mmol) in 1,4-dioxane (11.87 mL) is stirred at 90° C. for 8 hours. Then, crude was cooled down at room temperature and solvent was evaporated, and crude was purified using automated flash column chromatography using EtOAc and petroleum ether as eluent system to give 1,2-dimethyl-5-(4,4,5,5-tetramethyl-1,3,2-dioxaborolan-2-yl)benzimidazole (1.16 g, 4.25 mmol, 96% yield) as a brown powder.

UPLC-MS (ES+, Method 3): 1.35 min, m/z 273.1 [M+H]⁺

¹H-NMR (400 MHz, DMSO-d₆) δ/ppm: 7.81 (1H, t, J=0.9 Hz), 7.49 (2H, qd, J=8.1 Hz, 1.0 Hz), 3.73 (3H, s), 3.33 (3H, s), 1.31 (12H, s).

### Intermediate 5—1-methyl-5-(4,4,5,5-tetramethyl-1,3,2-dioxaborolan-2-yl)indazole

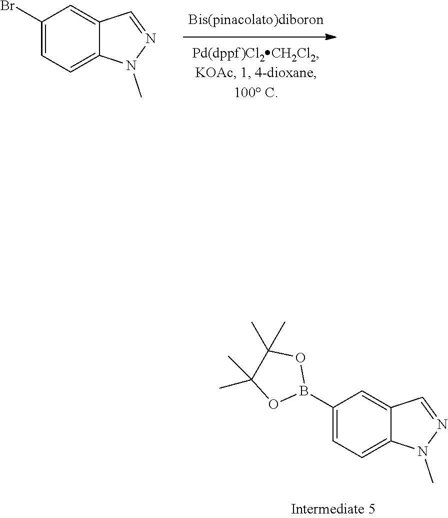 [SMILES: Cn1ncc2cc(Br)ccc21 ; Cn1ncc2cc(B3OC(C)(C)C(C)(C)O3)ccc21]

### 1-methyl-5-(4,4,5,5-tetramethyl-1,3,2-dioxaborolan-2-yl)indazole

5-Bromo-1-methyl-1H-indazole (6.9 g, 32.69 mmol), bis(pinacolato)diboron (10.79 g, 42.5 mmol), Pd(dppf)Cl₂·CH₂Cl₂ (2.67 g, 3.27 mmol), KOAc (6.42 g, 65.38 mmol) and 1,4-dioxane (200 mL). The reaction was degassed and flushed with nitrogen 3 times. It was heated to 100° C. and stirred for 2 hours. It was then cooled to room temperature and solvent removed in vacuo. DCM (50 mL) was added to the residue and the resulting mixture filtered through celite. The filtrate was concentrated in vacuo and residue purified by column chromatography using an eluent of 0-100% EtOAc in petroleum ether to give 1-methyl-5-(4,4,5,5-tetramethyl-1,3,2-dioxaborolan-2-yl)indazole (8.4 g, 32.54 mmol, 99% yield) as a yellow solid.

UPLC-MS (ES+, Method 1): 1.87 min, m/z 259.1 [M+H]⁺

¹H-NMR (400 MHz, CDCl₃) δ/ppm: 8.28 (1H, m), 7.99 (1H, d, J=0.8 Hz), 7.89 (1H, dd, J=4.4 Hz, 0.8 Hz), 7.36-7.40 (1H, m), 4.08 (3H, s), 1.37 (12H, s).

### Intermediate 6—2-[3-(methoxymethoxy)-1-naphthyl]-4,4,5,5-tetramethyl-1,3,2-dioxaborolane

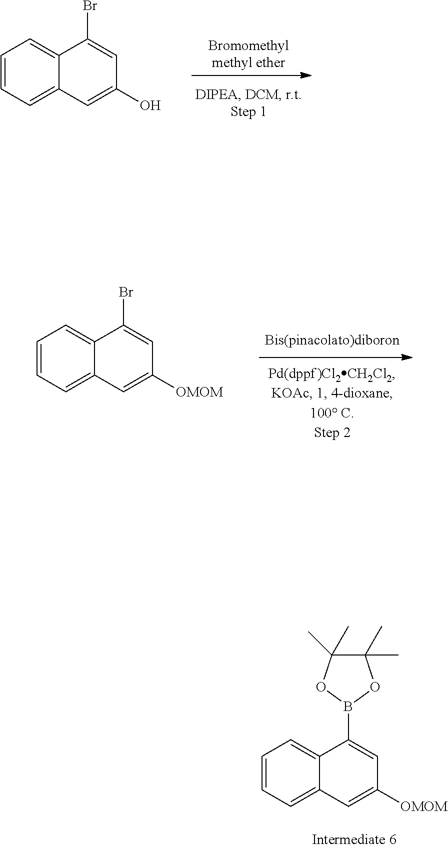

### 1-bromo-3-(methoxymethoxy)naphthalene

Bromomethyl methyl ether (7.32 mL, 89.66 mmol) was added slowly to a stirred solution of 1-bromo-3-hydroxynaphthalene (1 Og, 44.83 mmol), DIPEA (23.42 mL, 134.49 mmol) and DCM (400 mL) with ice cooling under a nitrogen atmosphere. The reaction was allowed to warm to room temperature and stir for 2 hours after which time it was complete by LCMS. The reaction was then poured into water (300 mL). The organic layer was separated, washed with sat. aq. NaHCO₃ (300 mL), sat. aq. NH₄Cl (300 mL), passed through a phase separator and concentrated in vacuo. The residue was purified by column chromatography using an eluent of 0-100% EtOAc in petroleum ether to give 1-bromo-3-(methoxymethoxy)naphthalene (11 g, 41.18 mmol, 92% yield) as a yellow oil.

UPLC-MS (ES+, Method 1): 2.13 min m/z 266.9, 268.9 [M+H]⁺

¹H-NMR (400 MHz, CDCl₃) δ/ppm: 8.17-8.17 (1H, m), 7.75-7.70 (1H, m), 7.58-7.56 (1H, m), 7.50-7.43 (2H, m), 7.40-7.37 (1H, m), 5.28 (2H, s), 3.52 (3H, s).

### 2-[3-(methoxymethoxy)-1-naphthyl]-4,4,5,5-tetramethyl-1,3,2-dioxaborolane

Bis(pinacolato)diboron (7.99 g, 31.45 mmol), 1-bromo-3-(methoxymethoxy)naphthalene (7.0 g, 26.21 mmol), KOAc (6.43 g, 65.51 mmol), Pd(dppf)Cl₂·CH₂Cl₂ (2.14 g, 2.62 mmol) and 1,4-dioxane (100 mL) were combined and stirred at room temperature. The reaction was fitted with a nitrogen balloon, evacuated and flushed with nitrogen 3 times and then heated to 100° C. for 1 hour. The reaction was then cooled to r.t. and solvent removed in vacuo. DCM (100 mL) was added to the residue and the mixture filtered through celite and the filter cake washed with DCM (100 mL). The filtrate was concentrated in vacuo and the residue purified by column chromatography using an eluent of 0-100% EtOAc in petroleum ether to give 2-[3-(methoxymethoxy)-1-naphthyl]-4,4,5,5-tetramethyl-1,3,2-dioxaborolane (6.8 g, 21.64 mmol, 82% yield) as a yellow solid.

UPLC-MS (ES+, Method 1): 1.52 min, m/z 232.2 [M-(Me₂CCMe₂)+H]⁺

¹H-NMR (400 MHz, CDCl₃) δ/ppm: 8.69-8.63 (1H, m), 7.79 (1H, d, J=2.4 Hz), 7.75-7.71 (1H, m), 7.47 (1H, d, J=2.4 Hz), 7.45-7.37 (2H, m), 5.30 (2H, s), 3.52 (3H, s), 1.42 (12H, s).

### Intermediate 7—1,3-dimethyl-5-(4,4,5,5-tetramethyl-1,3,2-dioxaborolan-2-yl)indazole

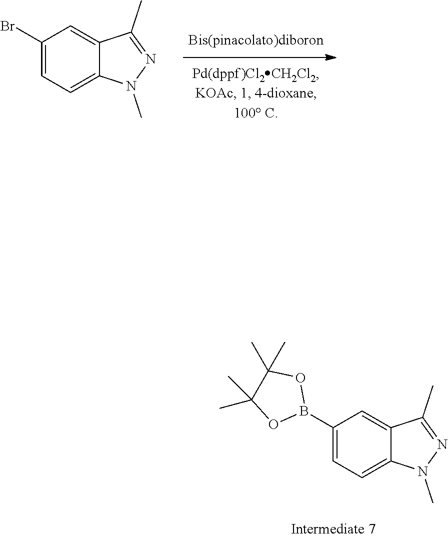 [SMILES: Cc1nn(C)c2ccc(Br)cc12 ; Cc1nn(C)c2ccc(B3OC(C)(C)C(C)(C)O3)cc12]

### 1,3-dimethyl-5-(4,4,5,5-tetramethyl-1,3,2-dioxaborolan-2-yl)indazole

Bis(pinacolato)diboron (677 mg, 2.67 mmol), Pd(dppf)Cl₂·CH₂Cl₂ (181 mg, 0.22 mmol), KOAc (0.44 g, 4.44 mmol), 5-bromo-1,3-dimethyl-1H-indazole (500 mg, 2.22 mmol) in 1,4-dioxane (5.93 mL) were combined and stirred at 90° C. for 6 hours. Then, crude was cooled down at room temperature, filtered through a Celite pad, solvent was evaporated and crude was purified using automated column chromatography to give 1,3-dimethyl-5-(4,4,5,5-tetramethyl-1,3,2-dioxaborolan-2-yl)indazole (600 mg, 2.09 mmol, 94% yield) as a yellow oil.

UPLC-MS (ES+, Method 3): 1.98 min, m/z 273.1 [M+H]⁺

¹H-NMR (400 MHz, CDCl₃) δ/ppm: 8.19 (1H, t, J=1.0 Hz), 7.79 (1H, dd, J=8.5 Hz, 1.0 Hz), 7.43-7.29 (1H, m), 4.00 (3H, s), 2.58 (3H, s), 1.38 (12H, s).

### Intermediate 8—5-(4,4,5,5-tetramethyl-1,3,2-dioxaborolan-2-yl)-1,3-benzoxazole

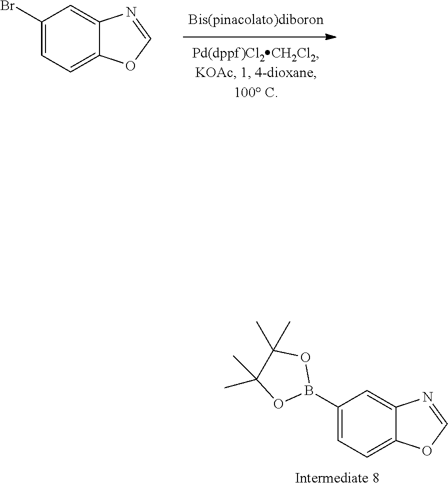 [SMILES: Brc1ccc2ocnc2c1 ; CC1(C)OB(c2ccc3ocnc3c2)OC1(C)C]

### 5-(4,4,5,5-tetramethyl-1,3,2-dioxaborolan-2-yl)-1,3-benzoxazole

Bis(pinacolato)diboron (769 mg, 3.03 mmol), Pd(dppf)Cl₂·CH₂Cl₂ (206 mg, 0.25 mmol), KOAc (0.5 g, 5.05 mmol), 5-bromo-1,3-benzoxazole (500 mg, 2.52 mmol) in 1,4-dioxane (10.1 mL) is stirred at 90° C. for 8 hours. Then, crude was cooled down at room temperature, filtered through a celite pad, solvent was evaporated, and crude was purified using automated column chromatography to give 5-(4,4,5,5-tetramethyl-1,3,2-dioxaborolan-2-yl)-1,3-benzoxazole (520 mg, 2.12 mmol, 84% yield) as a brown powder.

UPLC-MS (ES+, Method 3): 1.84 min, m/z 246.1 [M+H]⁺

¹H-NMR (400 MHz, CDCl₃) δ/ppm: 8.26 (1H, d, J=1.0 Hz), 8.09 (1H, s), 7.85 (1H, dd, J=8.2 Hz, 1.1 Hz), 7.58 (1H, dd, J=8.2 Hz, 0.7 Hz), 7.26 (1H, s), 1.37 (12H, s).

### Intermediate 9—N,N-dimethyl-2-[4-(4,4,5,5-tetramethyl-1,3,2-dioxaborolan-2-yl)phenoxy]acetamide

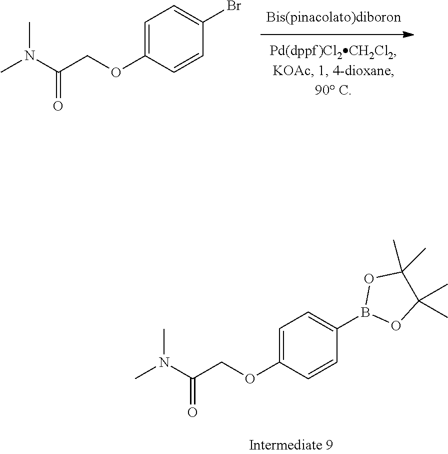

Bis(pinacolato)diboron (677 mg, 2.67 mmol), Pd(dppf)Cl₂·CH₂Cl₂ (181 mg, 0.22 mmol), KOAc (0.44 g, 4.44 mmol), 2-(4-bromophenoxy)-N,N-dimethyl-acetamide (573 mg, 2.22 mmol) in 1,4-dioxane (6.9 mL) is stirred at 90° C. for 8 hours. The reaction was then cooled to room temperature, filtered through a celite pad and solvent evaporated in vacuo. The residue was purified using automated column chromatography using 0-30% EtOAc in petroleum ether to give N,N-dimethyl-2-[4-(4,4,5,5-tetramethyl-1,3,2-dioxaborolan-2-yl)phenoxy]acetamide (540 mg, 1.68 mmol, 76% yield) as a yellow powder.

UPLC-MS (ES⁺, Method 3): 1.75 min, m/z 306.1 [M+H]⁺

¹H-NMR (400 MHz, CDCl₃) δ/ppm: 7.83-7.65 (2H, m), 7.02-6.81 (2H, m), 4.71 (2H, s), 3.08 (3H, s), 2.97 (3H, s), 1.33 (12H, s).

### Intermediate 10—1-morpholino-2-[4-(4,4,5,5-tetramethyl-1,3,2-dioxaborolan-2-yl)phenoxy]ethanone

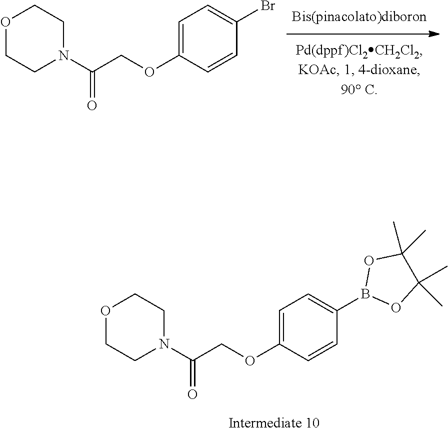 [SMILES: O=C(COc1ccc(Br)cc1)N1CCOCC1 ; CC1(C)OB(c2ccc(OCC(=O)N3CCOCC3)cc2)OC1(C)C]

Bis(pinacolato)diboron (487 mg, 1.92 mmol), Pd(dppf)Cl₂·CH₂Cl₂ (131 mg, 0.16 mmol), KOAc (310 mg, 3.2 mmol), 2-(4-bromophenoxy)-1-morpholino-ethanone (480 mg, 1.6 mmol) in 1,4-dioxane (5.7 mL) is stirred at 90° C. for 8 hours. The reaction was then cooled to room temperature, filtered through a celite pad and solvent evaporated in vacuo. The residue was purified using automated column chromatography using 0-30% EtOAc in petroleum ether to give 1-morpholino-2-[4-(4,4,5,5-tetramethyl-1,3,2-dioxaborolan-2-yl)phenoxy]ethanone (300 mg, 0.864 mmol, 54% yield) as a brown powder.

UPLC-MS (ES+, Method 3): 1.75 min, m/z 348.2 [M+H]⁺

¹H-NMR (400 MHz, CDCl₃) δ/ppm: 7.83-7.74 (2H, m), 6.98-6.90 (2H, m), 4.72 (2H, s), 3.68-3.56 (8H, m), 1.33 (12H, s).

### Intermediate 11—tert-butyl-6-[4-bromo-1-methyl-5-(5-methyl-1-tetrahydropyran-2-yl-indazol-4-yl)imidazol-2-yl]-2-azaspiro[3.3]heptane-2-carboxylate

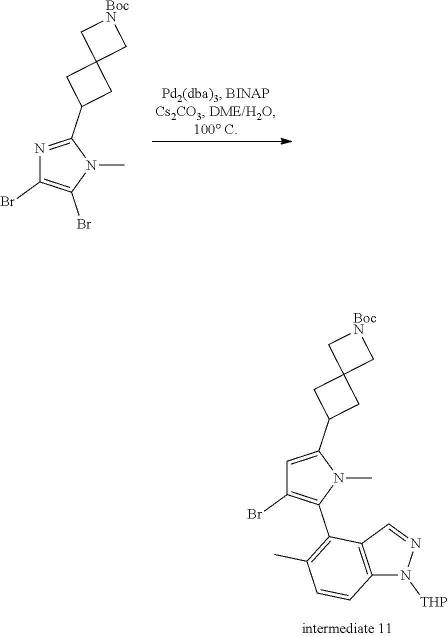

### tert-butyl-6-[4-bromo-1-methyl-5-(5-methyl-1-tetrahydropyran-2-yl-indazol-4-yl)imidazol-2-yl]-2-azaspiro[3.3]heptane-2-carboxylate

tert-butyl-6-(4,5-dibromo-1-methyl-imidazol-2-yl)-2-azaspiro[3.3]heptane-2-carboxylate (220 mg, 0.51 mmol), Intermediate 2 (208 mg, 0.61 mmol), Cs₂CO₃ (1.32 g, 4.04 mmol), Pd₂(dba)₃ (69 mg, 0.08 mmol), (+/−)-BINAP (47 mg, 0.08 mmol) in DME (8.41 mL) and water (4.22 mL) were stirred at 110° C. for one hour in a sealed microwave vial under conventional heating conditions. Product was observed after 1 hour. The crude was cooled to room temperature and filtered through a celite pad, solvent was evaporated and the crude residue was purified using automated flash column chromatography using EtOAc and petroleum ether as eluent system (flushing out the column using DCM/MeOH) to give the desired tert-butyl-6-[4-bromo-1-methyl-5-(5-methyl-1-tetrahydropyran-2-yl-indazol-4-yl)imidazol-2-yl]-2-azaspiro[3.3]heptane-2-carboxylate (100 mg, 0.175 mmol, 35% yield) as a yellow oil.

UPLC-MS (ES+, Method 3): 2.13 min, m/z 572.2 [M+H]⁺

### Intermediate 12—tert-butyl 6-[4-bromo-5-[3-(methoxymethoxy)-1-naphthyl]-1-methyl-imidazol-2-yl]-2-azaspiro[3.3]heptane-2-carboxylate

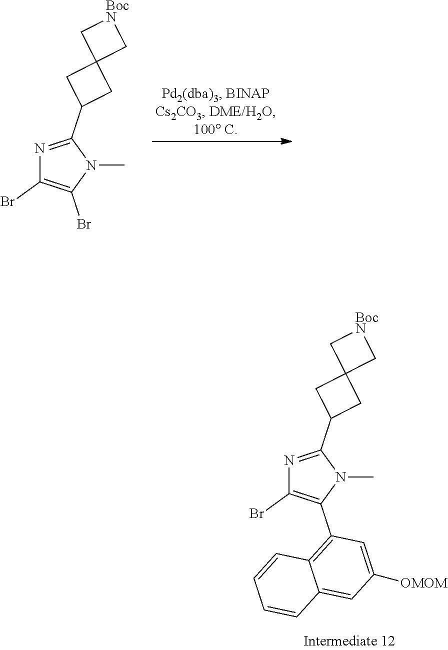

### tert-butyl 6-[4-bromo-5-[3-(methoxymethoxy)-1-naphthyl]-1-methyl-imidazol-2-yl]-2-azaspiro[3.3]heptane-2-carboxylate

(+/−)-BINAP (86 mg, 0.14 mmol), Intermediate 6 (346 mg, 1.1 mmol), Cs₂CO₃ (2.4 g, 7.35 mmol), Intermediate 1 (400 mg, 0.92 mmol), Pd₂(dba)₃ (127 mg, 0.14 mmol), DME (3 mL) and water (1 mL) were combined in a sealable vial and stirred. The mixture was bubbled through with nitrogen for 1 min and then the vial sealed and heated to 110° C. for 1 hour. It was then cooled to room temperature and solvent removed in vacuo. The residue was taken up in DCM (5 mL) and filtered through celite and the filter cake washed with DCM (5 mL). The filtrate was concentrated in vacuo and the residue purified by column chromatography using an eluent of 0-100% EtOAc in petroleum ether to give tert-butyl 6-[4-bromo-5-[3-(methoxymethoxy)-1-naphthyl]-1-methyl-imidazol-2-yl]-2-azaspiro[3.3]heptane-2-carboxylate (350 mg, 0.65 mmol, 70% yield) as a yellow solid.

UPLC-MS (ES+, Method 1): 2.04 min, m/z 542.5, 544.5 [M+H]⁺

### Intermediate 13—tert-butyl 6-(4,5-dibromo-1-ethyl-imidazol-2-yl)-2-azaspiro[3.3]heptane-2-carboxylate

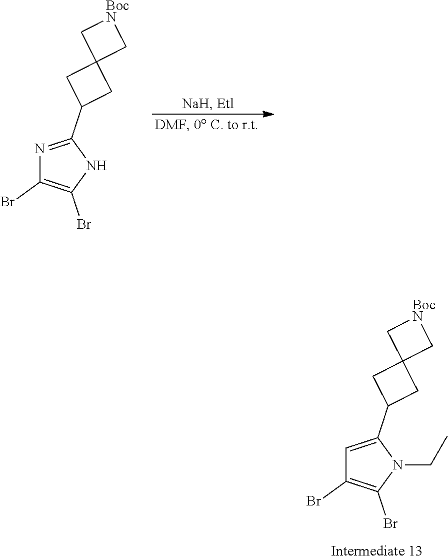 [SMILES: CC(C)(C)OC(=O)N1CC2(CC(c3nc(Br)c(Br)[nH]3)C2)C1 ; CCn1c(C2CC3(C2)CN(C(=O)OC(C)(C)C)C3)cc(Br)c1Br]

NaH (60% dispersed in mineral oil) (0.51 g, 12.74 mmol) was added to a stirred solution of Intermediate 1 (4.47 g, 10.61 mmol) and DMF (20 mL) in 2 portions over 20 minutes with ice cooling under a nitrogen atmosphere. After stirring for 10 minutes EtI (0.94 mL, 11.68 mmol) was added and the reaction allowed to warm to r.t. and stirred for 1.5 h. It was then poured into sat. aq. NaHCO₃ (100 mL) and the resulting mixture extracted with EtOAc (2×100 mL). The combined organic layers were dried over Na₂SO₄ and solvent removed in vacuo. The residue was purified by column chromatography using an eluent of 0-100% EtOAC in petroleum ether to give tert-butyl 6-(4,5-dibromo-1-ethyl-imidazol-2-yl)-2-azaspiro[3.3]heptane-2-carboxylate (2.7 g, 6.01 mmol, 57% yield) as a yellow solid.

UPLC-MS (ES+, Method 1): 1.99 min, m/z 448.0, 450.0, 452.0 [M+H]⁺

¹H-NMR (400 MHz, CDCl₃) δ/ppm: 4.04 (2H, s), 3.92-2.84 (4H, m), 3.35 (1H, pentet, J=8.4 Hz), 2.69-2.61 (2H, m), 2.54-2.47 (2H, m), 1.43 (9H, s), 1.30-1.23 (3H, m).

### Intermediate 14-tert-butyl 6-[4-bromo-1-ethyl-5-(5-methyl-1-tetrahydropyran-2-yl-indazol-4-yl)imidazol-2-yl]-2-azaspiro[3.3]heptane-2-carboxylate

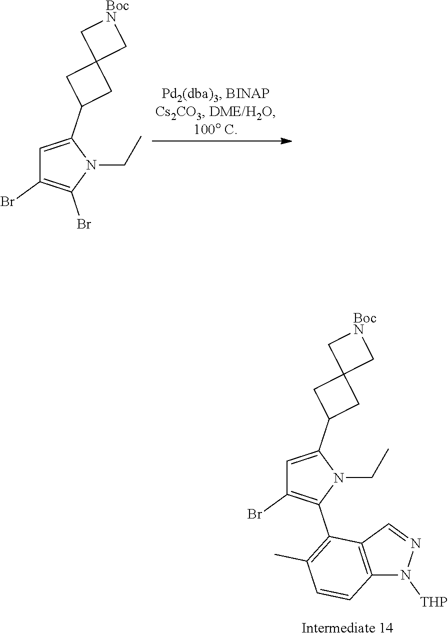

(+/−)-BINAP (35 mg, 0.06 mmol), Intermediate 2 (155 mg, 0.45 mmol), Cs₂CO₃ (370 mg, 1.14 mmol), Intermediate 13 (170 mg, 0.38 mmol), Pd₂(dba)₃ (52 mg, 0.06 mmol), monoglyme (3 mL) and water (1 mL) were combined in a sealable vial and stirred. The mixture was bubbled through with nitrogen for 1 minute and then the vial sealed and heated to 100° C. for 18 hours. It was then cooled to room temperature, combined and solvent removed in vacuo. The residue was taken up in DCM (5 mL) and filtered through celite and the filter cake washed with DCM (5 mL). The filtrate was concentrated in vacuo and the residue purified by column chromatography using an eluent of 0-100% EtOAc in petroleum ether to give tert-butyl 6-[4-bromo-1-ethyl-5-(5-methyl-1-tetrahydropyran-2-yl-indazol-4-yl)imidazol-2-yl]-2-azaspiro[3.3]heptane-2-carboxylate (75 mg, 0.13 mmol, 34% yield) as a yellow solid.

UPLC-MS (ES+, Method 3): 2.17 min, m/z 584.2, 586.2 [M+H]⁺

### Intermediate 15—tert-butyl 6-[4-bromo-1-ethyl-5-[3-(methoxymethoxy)-1-naphthyl]imidazol-2-yl]-2-azaspiro[3.3]heptane-2-carboxylate

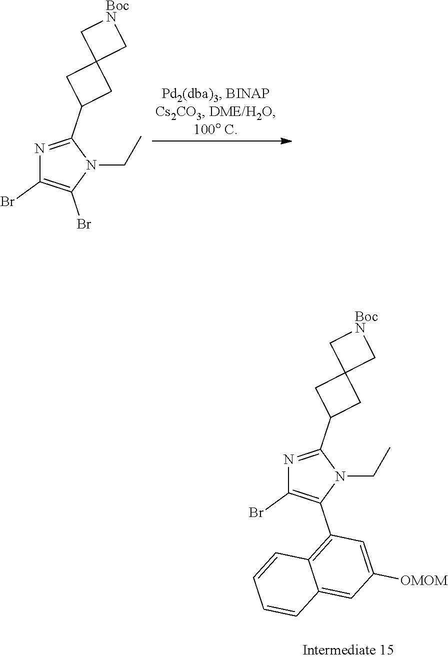

Intermediate 6 (419 mg, 1.34 mmol), Intermediate 13 (500 mg, 1.12 mmol), (+/−)-BINAP (104 mg, 0.16 mmol), Pd₂(dba)₃ (152 mg, 0.16 mmol), Cs₂CO₃ (1.08 g, 3.34 mmol), monoglyme (9 mL) and water (1 mL) were combined and stirred in a sealable vial. Nitrogen was bubbled through the reaction mixture for 1 minute and then the vial sealed and stirred at 110° C. for for 6 hours. The reaction was then cooled to room temperature and solvent removed in vacuo. The residue was taken up in DCM (20 mL). and the mixture filtered through celite and the filter cake washed with DCM (10 mL). The filtrate was concentrated in vacuo and the residue purified by column chromatography using an eluent of 0-100% EtOAc in petroleum ether to give tert-butyl 6-[4-bromo-1-ethyl-5-[3-(methoxymethoxy)-1-naphthyl]imidazol-2-yl]-2-azaspiro[3.3]heptane-2-carboxylate (400 mg, 0.7188 mmol, 64% yield) a yellow oil.

UPLC-MS (ES+, Method 1): 2.09 min, m/z 556.5, 558.5 [M+H]⁺

### Intermediate 16—5,6-dimethyl-1-tetrahydropyran-2-yl-4-(4,4,5,5-tetramethyl-1,3,2-dioxaborolan-2-yl)indazole

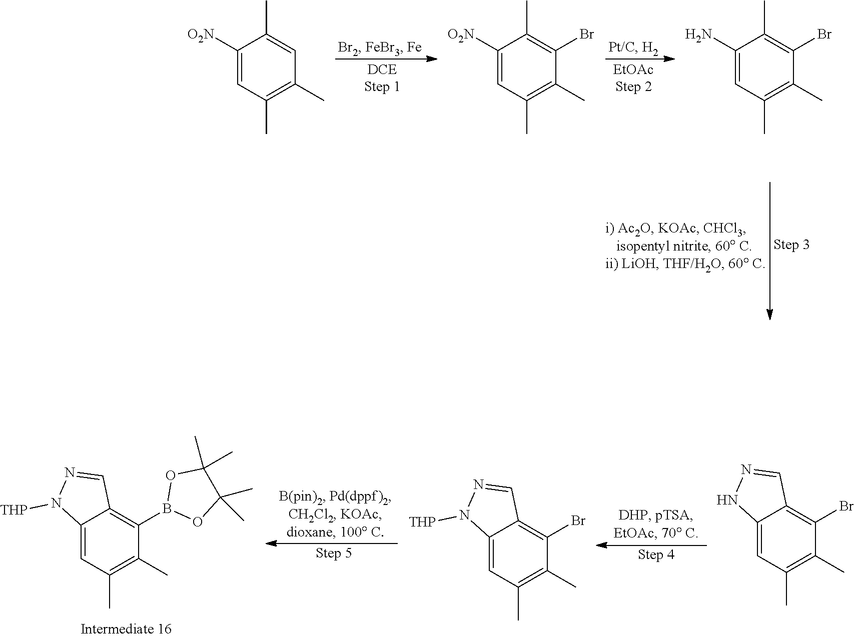

### Step 1—3-bromo-1,2,4-trimethyl-5-nitro-benzene

To a solution of 1,2,4-trimethyl-5-nitrobenzene (10 g, 60.54 mmol) in DCE (200 mL) was added FeBr₃ (358 mg, 1.21 mmol), iron (845 mg, 15.13 mmol) and Br₂ (3.72 mL, 72.64 mmol). The mixture was stirred at room temperature for 18 hours and then diluted with water (200 mL) and extracted with DCM (3×100 mL). The combined organic layers was washed with brine (200 mL), dried over Na₂SO₄, filtered and concentrated under reduced pressure. The crude residue was triturated with water and collected by filtration to give 3-bromo-1,2,4-trimethyl-5-nitro-benzene (14.2 g, 58.175 mmol, 96% yield) as a white solid.

UPLC-MS (ES+, Method 1), 2.12 min, no mass ion seen, no ionizable centre ¹H-NMR (400 MHz, DMSO-d₆) δ/ppm: 7.75 (1H, s), 2.46 (3H, s), 2.43 (3H, s), 2.38 (3H, s).

### Step 2—3-bromo-2,4,5-trimethyl-aniline

Pt/C, 5 wt. % loading (820 mg, 4.2 mmol) was added to a stirred solution of 3-bromo-1,2,4-trimethyl-5-nitro-benzene (8.2 g, 33.59 mmol) and EtOAc (100 mL) at room temperature under a nitrogen atmosphere. The reaction was fitted with a hydrogen balloon and subjected to 3× vacuum/hydrogen cycles and then allowed to stir under a nitrogen atmosphere for 18 hours. The reaction was then filtered through celite and the filter cake washed with EtOAc (100 mL). The filtrate was then concentrated in vacuo to give 3-bromo-2,4,5-trimethyl-aniline (7.1 g, 33.16 mmol, 99% yield) as a brown solid which was used in the next step without further purification.

UPLC-MS (ES+, Method 1): 1.64 min, m/z 213.9, 215.9 [M+H]⁺

¹H-NMR (400 MHz, CDCl₃) δ/ppm: 6.50 (1H, s), 2.29 (3H, s), 2.28 (3H, s), 2.23 (3H, s), exchangeable NH₂ not seen.

### Step 3—4-bromo-5,6-dimethyl-1H-indazole

Acetic anhydride (15.02 mL, 159.04 mol) was added dropwise to a stirred solution of 3-bromo-2,4,5-trimethyl-aniline (11.35 g, 53.01 mmol), CHCl₃ (140 mL) and KOAc (6.24 g, 63.61 mmol) with ice cooling under a nitrogen atmosphere. The reaction was then warmed to 60° C. and isopentyl nitrite (14.24 mL, 106.2 mmol) was added slowly. The resulting mixture was then allowed to stir for 18 hours and then cooled to room temperature and partitioned between water (200 mL) and DCM (200 mL). The organic layer was separated and concentrated in vacuo. The residue was stirred in THE (140 mL) and Water (70 mL) with LiOH (8.89 g, 371.08 mmol) at 60° C. for 48 hours. Solvent was then removed in vacuo and the partitioned between sat. aq. NH₄Cl (200 mL) and DCM (200 mL). The organic layer was separated and the aqueous layer extracted with DCM (200 mL). The residue was stirred in diethyl ether with ice cooling and the resulting solid filtered and dried in vacuo to give 4-bromo-5,6-dimethyl-1H-indazole (11.5 g, 51.09 mmol, 96% yield) as a brown solid.

UPLC-MS (ES+, Method 1): 1.76 min, m/z 224.8, 226.8 [M+H]⁺

¹H-NMR (400 MHz, DMSO-d₆) δ/ppm: 13.20 (1H, br s), 7.88 (1H, s), 7.37 (1H, s), 2.42 (3H, s), 2.39 (3H, s).

### Step 4—4-bromo-5,6-dimethyl-1-tetrahydropyran-2-yl-indazole

DHP (6.99 mL, 76.64 mmol) was added to a stirred solution of 4-bromo-5,6-dimethyl-1H-indazole (11.5 g, 51 mmol) and p-toluenesulfonic acid monohydrate (486 mg, 2.55 mmol) in EtOAc (250 mL) and the mixture was heated at 70° C. for 6 hours. The mixture was the cooled to room temperature and poured into saturated aqueous sodium bicarbonate (200 mL) and the phases were separated. The aqueous phase was extracted with EtOAc (200 mL) and the combined organic layers were dried over sodium sulfate, filtered and concentrated in vacuo. The residue was purified by column chromatography, using an eluent of 0-50% EtOAc in petroleum ether to give 4-bromo-5,6-dimethyl-1-tetrahydropyran-2-yl-indazole (3.2 g, 10.35 mmol, 20% yield) as a yellow solid.

UPLC-MS (ES+, short acidic), 2.14 min, m/z 308.9, 310.9 [M+H]⁺

### Step 5—5,6-dimethyl-1-tetrahydropyran-2-yl-4-(4,4,5,5-tetramethyl-1,3,2-dioxaborolan-2-yl)indazole

Bis(pinacolato)diboron (1.97 g, 7.76 mmol), 4-bromo-5,6-dimethyl-1-tetrahydropyran-2-yl-indazole (2.0 g, 6.47 mmol), KOAc (1.27 g, 12.94 mmol), Pd(dppf)Cl₂·CH₂Cl₂ (528 mg, 0.65 mmol) and 1,4-dioxane (50 mL) were combined and stirred at room temperature. The reaction was fitted with a nitrogen balloon, evacuated and flushed with nitrogen 3 times and then heated to 100° C. for 2 hours. The reaction was then cooled to r.t. and solvent removed in vacuo. DCM (10 mL) was added to the residue and the mixture filtered through celite and the filter cake washed with DCM (10 mL). The filtrate was concentrated in vacuo and the residue purified by column chromatography using an eluent of 0-100% EtOAc in petroleum ether to give 5,6-dimethyl-1-tetrahydropyran-2-yl-4-(4,4,5,5-tetramethyl-1,3,2-dioxaborolan-2-yl)indazole (1.4 g, 3.93 mmol, 61% yield) as a yellow solid.

UPLC-MS (ES+, Method 1): 2.22 min, m/z 357.2 [M+H]⁺

¹H-NMR (400 MHz, CDCl₃) δ/ppm: 8.28 (1H, d, J=5.2 Hz), 7.44 (1H, d, J=7.2 Hz), 5.70-5.66 (1H, m), 4.05-3.97 (1H, m), 3.77-3.69 (1H, m), 2.54 (3H, s), 2.40 (3H, s), 2.22-2.11 (1H, m), 2.06-2.01 (1H, m), 1.81-1.62 (4H, m), 1.41 (12H, s).

### Intermediate 17—2-[5-(methoxymethoxy)-2-(trifluoromethyl)phenyl]-4,4,5,5-tetramethyl-1,3,2-dioxaborolane

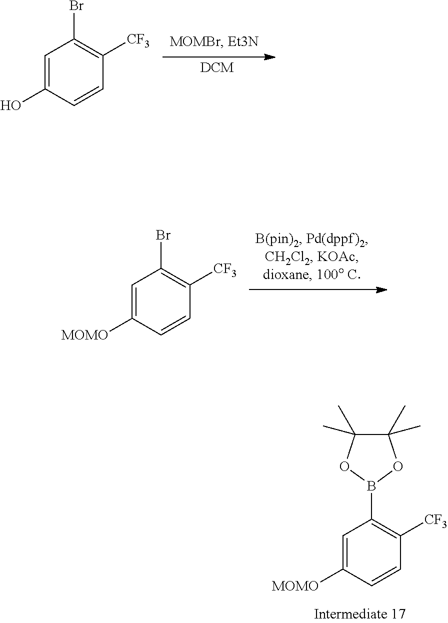

### Step 1—2-bromo-4-(methoxymethoxy)-1-(trifluoromethyl)benzene

A solution of 3-bromo-4-(trifluoromethyl)phenol (1 g, 4.15 mmol) in DCM (10 mL) is cooled down to 0° C. before adding Et₃N (1.73 mL, 12.45 mmol), followed by slow addition of bromomethyl methyl ether (0.68 mL, 8.3 mmol). The mixture is then stirred at room temperature for 1 hour. The mixture is washed with sat. aq. Na₂CO₃ and the organics dried with Na₂SO₄ and evaporated, yielding the crude product 2-bromo-4-(methoxymethoxy)-1-(trifluoromethyl)benzene (1.18 g, 4.15 mmol, 100% yield) as a yellow oil.

¹H-NMR (400 MHz, CDCl₃) δ/ppm: 7.58 (2H, d, J=8.9 Hz), 7.38 (1H, d, J=2.9 Hz), 7.04-7.01 (1H, m), 5.19 (2H, s), 3.47 (3H, s).

### Step 2—2-[5-(methoxymethoxy)-2-(trifluoromethyl)phenyl]-4,4,5,5-tetramethyl-1,3,2-dioxaborolane

To a flask is added 2-bromo-4-(methoxymethoxy)-1-(trifluoromethyl)benzene (730 mg, 2.56 mmol), bis(pinacolato)diboron (975 mg, 3.84 mmol), KOAc (754 mg, 7.68 mmol) and Pd(dppf)Cl₂·CH₂Cl₂ (188 mg, 0.26 mmol) in dry toluene (15 mL). The mixture is degassed with nitrogen and reacted at 100° C. for 2 hours. After this time extra bis(pinacolato)diboron (1.3 g, 5.12 mmol), KOAc (754 mg, 7.68 mmol) and Pd(dppf)Cl₂·CH₂Cl₂ (564 mg, 0.77 mmol) is added and the mixture is stirred at 100° C. for another 6 hours. After this time, starting material not detected by LCMS. The mixture was cooled to room temperature and filtered through celite with DCM and the filtrate concentrated in vacuo. The residue was purified by column chromatography using an eluent of 0-20% EtOAc in petroleum ether to give 2-[5-(methoxymethoxy)-2-(trifluoromethyl)phenyl]-4,4,5,5-tetramethyl-1,3,2-dioxaborolane (570 mg, 1.72 mmol, 67% yield) as a clear oil which solidified upon standing.

¹H-NMR (400 MHz, CDCl₃) δ/ppm: 7.59-7.53 (1H, m), 7.33-7.32 (1H, m), 7.14-7.10 (1H, m), 5.21 (2H, s), 3.46 (3H, s), 1.26 (12H, s).

### Intermediate 18—1-tetrahydropyran-2-yl-4-(4,4,5,5-tetramethyl-1,3,2-dioxaborolan-2-yl)indazole-6-carbonitrile

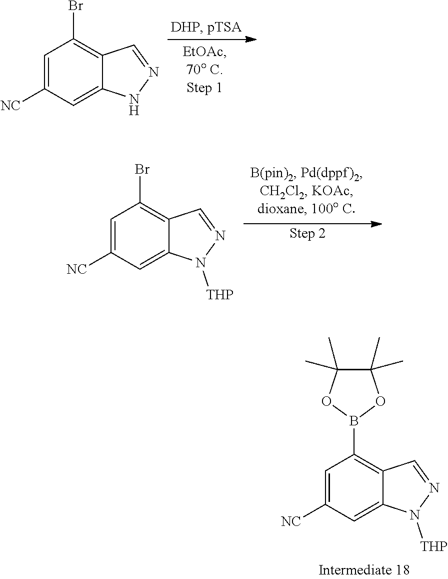

### Step 1—4-bromo-1-tetrahydropyran-2-yl-indazole-6-carbonitrile

DHP (0.82 mL, 9.01 mmol) was added to a stirred solution of 4-bromo-1H-indazole-6-carbonitrile (1 g, 4.5 mmol) and p-toluenesulfonic acid monohydrate (86 mg, 0.45 mmol) in EtOAc (50 mL) and the mixture was heated at 70° C. for 6 hours. The mixture was the cooled to room temperature and poured into saturated aqueous sodium bicarbonate (200 mL) and the phases were separated. The aqueous phase was extracted with EtOAc (200 mL) and the combined organic layers were dried over sodium sulfate, filtered and concentrated in vacuo. The residue was purified by column chromatography, using an eluent of 0-50% EtOAc in petroleum ether to give 4-bromo-1-tetrahydropyran-2-yl-indazole-6-carbonitrile (910 mg, 2.972 mmol, 66% yield) as a yellow solid.

UPLC-MS (ES+, Method 3), 2.03 min, m/z 305.9, 307.9 [M+H]⁺

¹H-NMR (400 MHz, CDCl₃,) δ/ppm: 8.09 (1H, d, J=0.8 Hz), 8.00 (1H, t, J=0.8 Hz), 7.53 (1H, d, J=1.2 Hz), 5.75 (1H, dd, J=8.8 Hz, 2.8 Hz), 4.03-3.96 (1H, m), 3.81-3.73 (1H, m), 2.51-2.40 (1H, m), 2.18-2.09 (2H, m), 1.85-1.68 (3H, m).

### Step 2—1-tetrahydropyran-2-yl-4-(4,4,5,5-tetramethyl-1,3,2-dioxaborolan-2-yl)indazole-6-carbonitrile

Bis(pinacolato)diboron (448 mg, 1.76 mmol), 4-bromo-1-tetrahydropyran-2-yl-indazole-6-carbonitrile (450 mg, 1.47 mmol), KOAc (288 mg, 2.94 mmol), Pd(dppf)Cl₂·CH₂Cl₂ (120 mg, 0.15 mmol) and 1,4-dioxane (5 mL) were combined and stirred at room temperature. The reaction was fitted with a nitrogen balloon, evacuated and flushed with nitrogen 3 times and then heated to 80° C. for 2 hours. The reaction was then cooled to r.t. and solvent removed in vacuo. DCM (10 mL) was added to the residue and the mixture filtered through celite and the filter cake washed with DCM (10 mL). The filtrate was concentrated in vacuo and the residue purified by column chromatography using an eluent of 0-100% EtOAc in petroleum ether to give 1-tetrahydropyran-2-yl-4-(4,4,5,5-tetramethyl-1,3,2-dioxaborolan-2-yl)indazole-6-carbonitrile (390 mg, 1.104 mmol, 75% yield) as a yellow solid.

UPLC-MS (ES+, Method 3): 1.58 min, m/z 272.1 [M-(Me2CCMe2)+H]⁺; 2.29 min, m/z 354.2, [M+H]⁺

¹H-NMR (400 MHz, CDCl₃) δ/ppm: 8.44 (1H, d J=0.8 Hz), 8.10 (1H, t, J=1.2 Hz), 7.86 (1H, d, J=1.2 Hz), 5.76 (1H, dd, J=8.8 Hz, 2.8 Hz), 4.02-3.97 (1H, m), 3.79-3.72 (1H, m), 2.55-2.42 (1H, m), 2.20-2.02 (2H, m), 1.83-1.65 (3H, m), 1.40 (12H, s).

### Intermediate 19—6-chloro-1-tetrahydropyran-2-yl-4-(4,4,5,5-tetramethyl-1,3,2-dioxaborolan-2-yl)indazole

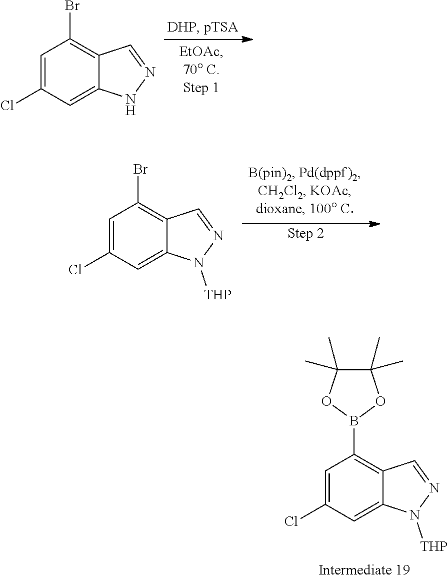

### Step 1—4-bromo-6-chloro-1-tetrahydropyran-2-yl-indazole

DHP (0.59 mL, 6.48 mmol) was added to a stirred solution of 4-bromo-6-chloro-1H-indazole (750 mg, 3.24 mmol) and p-toluenesulfonic acid monohydrate (62 mg, 0.32 mmol) in EtOAc (50 mL) and the mixture was heated at 70° C. for 6 hours. The mixture was the cooled to room temperature and poured into sat. aq. NaHCO₃ (200 mL) and the phases were separated. The aqueous phase was extracted with EtOAc (200 mL) and the combined organic layers were dried over sodium sulfate, filtered and concentrated in vacuo. The residue was purified by column chromatography, using an eluent of 0-50% EtOAc in petroleum ether to give 4-bromo-6-chloro-1-tetrahydropyran-2-yl-indazole (900 mg, 2.852 mmol, 88% yield) as a yellow solid.

UPLC-MS (ES+, Method 3), 2.25 min, m/z 314.9, 316.9, 318.9 [M+H]⁺

¹H-NMR (400 MHz, CDCl₃) δ/ppm: 7.98 (1H, d, J=0.8 Hz), 7.58 (1H, dd, J=1.2 Hz, 0.8 Hz), 7.33 (1H, d, J=1.2 Hz), 5.65 (1H, dd, J=9.2 Hz, 2.8 Hz), 4.04-3.96 (1H, m), 3.78-3.70 (1H, m), 2.54-2.44 (1H, m), 2.19-2.04 (2H, m), 1.82-1.63 (3H, m).

### Step 2—6-chloro-1-tetrahydropyran-2-yl-4-(4,4,5,5-tetramethyl-1,3,2-dioxaborolan-2-yl)indazole

Bis(pinacolato)diboron (434 mg, 1.71 mmol), 4-bromo-6-chloro-1-tetrahydropyran-2-yl-indazole (450 mg, 1.43 mmol), KOAc (280 mg, 2.85 mmol), Pd(dppf)Cl₂·CH₂Cl₂ (116 mg, 0.14 mmol) and 1,4-dioxane (10 mL) were combined and stirred at room temperature. The reaction was fitted with a nitrogen balloon, evacuated and flushed with nitrogen 3 times and then heated to 80° C. for 2 hours. The reaction was then cooled to r.t. and solvent removed in vacuo. DCM (10 mL) was added to the residue and the mixture filtered through celite and the filter cake washed with DCM (10 mL). The filtrate was concentrated in vacuo and the residue purified by column chromatography using an eluent of 0-100% EtOAc in petroleum ether to give 6-chloro-1-tetrahydropyran-2-yl-4-(4,4,5,5-tetramethyl-1,3,2-dioxaborolan-2-yl)indazole (490 mg, 1.351 mmol, 95% yield) as a yellow solid.

UPLC-MS (ES+, Method 3): 1.71 min, m/z 281.0, 283.0 [M-(Me2CCMe2)+H]⁺; 2.46 min, m/z 363.2, 365.2 [M+H]⁺

¹H-NMR (400 MHz, CDCl₃) δ/ppm: 8.34 (1H, d J=4.4 Hz), 7.70 (1H, d, J=5.6 Hz), 7.61 (1H, d, J=2.4 Hz), 5.67 (1H, dd, J=9.2 Hz, 2.8 Hz), 4.05-3.98 (1H, m), 3.78-3.70 (1H, m), 2.57-2.45 (1H, m), 2.20-2.01 (2H, m), 1.81-1.62 (3H, m), 1.39 (12H, s).

### Example 1—1-[6-[1-methyl-4-(1-methylindazol-5-yl)-5-(5-methyl-1H-indazol-4-yl)imidazol-2-yl]-2-azaspiro[3.3]heptan-2-yl]prop-2-en-1-one

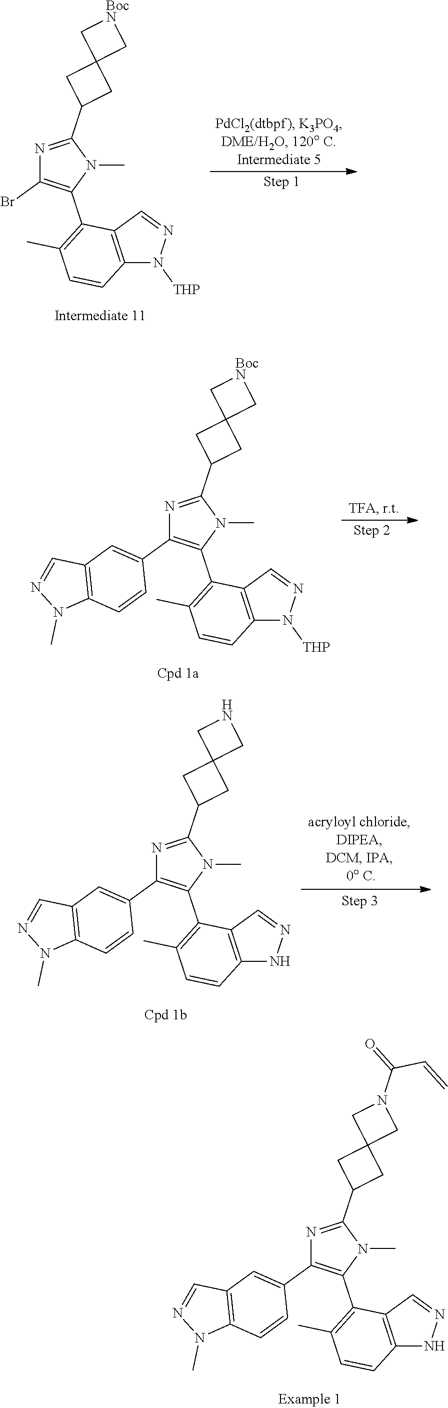

### Step 1: tert-butyl-6-[1-methyl-4-(1-methylindazol-5-yl)-5-(5-methyl-1-tetrahydropyran-2-yl-indazol-4-yl)imidazol-2-yl]-2-azaspiro[3.3]heptane-2-carboxylate

Intermediate 2 (45 mg, 0.18 mmol), K₃PO₄ (223 mg, 1.05 mmol), Intermediate 11 (100 mg, 0.18 mmol), PdCl₂(dtbpf) (3 mg, 0.004 mmol) in DME (3.41 mL) and water (1.0 mL) were stirred at 90° C. for 1 hour. The reaction mixture was then cooled to r.t., filtered through a celite pad, and solvent evaporated to give a brown oil which was purified via automated flash column chromatography using EtOAc in petroleum ether as an eluent system to give tert-butyl-6-[1-methyl-4-(1-methylindazol-5-yl)-5-(5-methyl-1-tetrahydropyran-2-yl-indazol-4-yl)imidazol-2-yl]-2-azaspiro[3.3]heptane-2-carboxylate (70 mg, 0.11 mmol, 64% yield) as a yellow oil.

UPLC-MS (ES+, Method 1): 1.74 min, m/z 622.4 [M+H]⁺

### Step 2: 4-(2-{2-azaspiro[3.3]heptan-6-yl}-1-methyl-4-(1-methyl-1H-indazol-5-yl)-1H-imidazol-5-yl)-5-methyl-1H-indazole

Tert-butyl-6-[1-methyl-4-(1-methylindazol-5-yl)-5-(5-methyl-1-tetrahydropyran-2-yl-indazol-4-yl)imidazol-2-yl]-2-azaspiro[3.3]heptane-2-carboxylate (70 mg, 0.11 mmol) and TFA (1.0 mL, 13.06 mmol) were stirred at ˜10° C. (water and ice bath) for 5 min. Then, the solvent was evaporated and crude 4-[2-(2-azaspiro[3.3]heptan-6-yl)-3-methyl-5-(1-methylindazol-5-yl)imidazol-4-yl]-5-methyl-1H-indazole (40 mg, 0.091 mmol, 81% yield) was used directly in the next step.

UPLC-MS (ES+, Method 3): 1.23 min, m/z 438.23 [M+H]⁺

### Step 3: 1-{6-[1-methyl-5-(5-methyl-1H-indazol-4-yl)-4-(1-methyl-1H-indazol-5-yl)-1H-imidazol-2-yl]-2-azaspiro[3.3]heptan-2-yl}prop-2-en-1-one—Example 1

4-[2-(2-azaspiro[3.3]heptan-6-yl)-3-methyl-5-(1-methylindazol-5-yl)imidazol-4-yl]-5-methyl-1H-indazole (70 mg, 0.16 mmol), DIPEA (0.08 mL, 0.48 mmol) in DCM (2.3 mL) and 1-propanol (0.9 mL) were stirred at 0° C. A solution of acryloyl chloride (0.01 mL, 0.16 mmol) in DCM (0.75 mL) was added to the reaction mixture and stirred at 0° C. for 5 min. Then, upon completion of reaction, reaction was quenched by adding 7N NH₃ in MeOH (10 mL) to the crude and reaction mixture was stirred at 0° C. for 30 min. The reaction mixture was then diluted with water (10 mL) and passed through a phase separator, solvent was evaporated and crude was purified using basic alumina flash column chromatography using DCM/MeOH as eluent system to give 1-[6-[1-methyl-4-(1-methylindazol-5-yl)-5-(5-methyl-1H-indazol-4-yl)imidazol-2-yl]-2-azaspiro[3.3]heptan-2-yl]prop-2-en-1-one (2 mg, 0.004 mmol, 2% yield) as a white powder.

UPLC-MS (ES+, Method 2): 2.65 min, m/z 492.2 [M+H]⁺

¹H-NMR (400 MHz, CDCl₃) δ/ppm: 7.79-7.70 (2H, m), 7.66 (1H, t, J=1.8 Hz), 7.52 (1H, d, J=8.5 Hz), 7.45 (1H, td, J=8.8 Hz, 1.6 Hz), 7.37 (1H, d, J=8.6 Hz), 7.17-7.05 (1H, m), 6.41-6.26 (1H, m), 6.22 (1H, ddd, J=16.9 Hz, 10.2 Hz, 6.2 Hz), 5.73-5.63 (1H, m), 4.38 (1H, d, J=8.1 Hz), 4.26 (2H, d, J=5.1 Hz), 4.15 (1H, d, J=3.2 Hz), 4.10-4.01 (1H, m), 3.97 (3H, s), 3.67-3.51 (2H, m), 3.17 (3H, d, J=3.5 Hz), 2.72 (1H, td, J=8.2 Hz, 3.9 Hz), 2.14 (3H, s), 0.94 (1H, td, J=7.3 Hz, 3.6 Hz). NH proton not visible in Chloroform.

In a similar manner to Step 1 in Example 1, starting from Intermediates 11 and 14; the following compounds in Table 4 were made using either intermediates 3, 4, 7, 8, 9 and 10 or commercially available boronates.

TABLE 4

| Compound | Structure | Yield | Data |
|---|---|---|---|
| 2a | tert-butyl 6-[1-methyl-5-(5-methyl-1- | (56 mg, | UPLC-MS |
| | tetrahydropyran-2-yl-indazol-4-yl)-4- | 0.099 mmol, | (ES+, Method |
| | phenyl-imidazol-2-yl]-2- | 51% yield) | 1): 1.67 min, |
| | azaspiro[3.3]heptane-2-carboxylate | | m/z 568.6 |
| | | | [M + H]⁺ |
| | 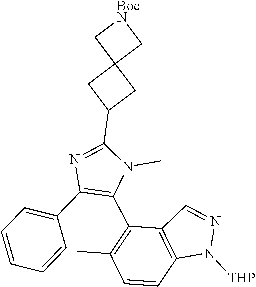 | | |
| 3a | tert-butyl 6-[1-methyl-5-(5-methyl-1- | (30 mg, | UPLC-MS |
| | tetrahydropyran-2-yl-indazol-4-yl)-4-(3- | 0.053 mmol, | (ES+, Method |
| | pyridyl)imidazol-2-yl]-2- | 27% yield) | 1): 1.59 min, |
| | azaspiro[3.3]heptane-2-carboxylate | | m/z 569.5 |
| | | | [M + H]⁺ |
| | 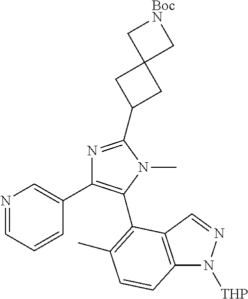 | | |
| 4a | tert-butyl 6-[4-(2-fluoro-4-methoxy- | (43 mg, | UPLC-MS |
| | phenyl)-1-methyl-5-(5-methyl-1- | 0.07 mmol, | (ES+, Method |
| | tetrahydropyran-2-yl-indazol-4- | 36% yield) | 1): 1.67 min, |
| | yl)imidazol-2-yl]-2-azaspiro[3.3]heptane- | | m/z 616.8 |
| | 2-carboxylate | | [M + H]⁺ |
| | 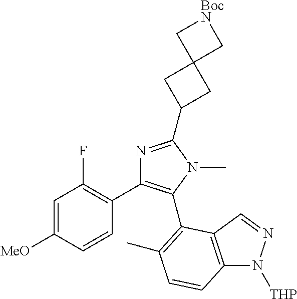 | | |
| 5a | tert-butyl 6-[1-methyl-5-(5-methyl-1- | (112 mg, | UPLC-MS |
| | tetrahydropyran-2-yl-indazol-4-yl)-4-(4- | 0.17 mmol, | (ES+, Method |
| | morpholinophenyl)imidazol-2-yl]-2- | 54% yield) | 1): 1.65 min, |
| | azaspiro[3.3]heptane-2-carboxylate | | m/z 653.6 |
| | | | [M + H]⁺ |
| | 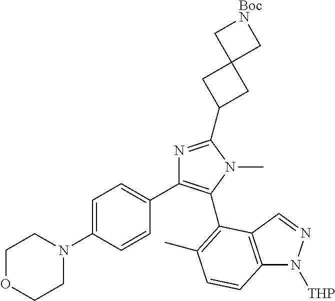 | | |
| 6a | tert-butyl 6-[1-methyl-5-(5-methyl-1- | (95 mg, | UPLC-MS |
| | tetrahydropyran-2-yl-indazol-4-yl)-4-(3- | 0.15 mmol, | (ES⁺, Method |
| | morpholinophenyl)imidazol-2-yl]-2- | 46% yield) | 1): 1.63 min, |
| | azaspiro[3.3]heptane-2-carboxylate | | m/z 653.5 |
| | | | [M + H]⁺ |
| | 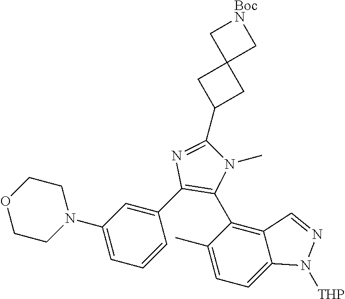 | | |
| 7a | tert-butyl 6-[1-methyl-4-(1-methylindazol- | (115 mg, | UPLC-MS |
| | 6-yl)-5-(5-methyl-1-tetrahydropyran-2-yl- | 0.185 mmol, | (ES⁺, Method |
| | indazol-4-yl)imidazol-2-yl]-2- | 58% yield) | 1): 1.63 min, |
| | azaspiro[3.3]heptane-2-carboxylate | | m/z 622.8 |
| | | | [M + H]⁺ |
| | 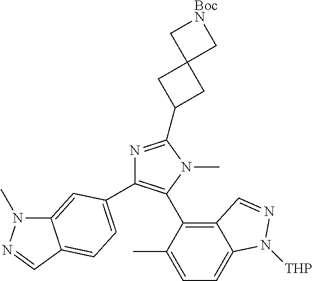 | | |
| 8a | tert-butyl-6-[4-[4-(4-cyclopropylimidazol- | (200 mg, | UPLC-MS |
| | 1-yl)phenyl]-1-methyl-5-(5-methyl-1- | 0.30 mmol, | (ES+, Method |
| | tetrahydropyran-2-yl-indazol-4- | 93% yield) | 1): 1.59 min, |
| | yl)imidazol-2-yl]-2-azaspiro[3.3]heptane- | | m/z 674.5 |
| | 2-carboxylate | | [M + H]⁺ |
| | 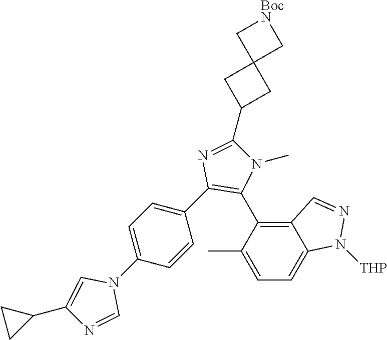 | | |
| 9a | tert-butyl-6-[4-(1,2-dimethylbenzimidazol- | (55 mg, | UPLC-MS |
| | 5-yl)-1-methyl-5-(5-methyl-1- | 0.086 mmol, | (ES+, Method |
| | tetrahydropyran-2-yl-indazol-4- | 33% yield) | 1): 1.55 min, |
| | yl)imidazol-2-yl]-2-azaspiro[3.3]heptane- | | m/z 636.6 |
| | 2-carboxylate | | [M + H]⁺ |
| | 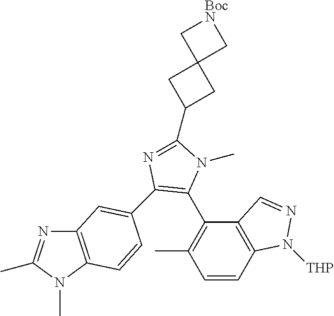 | | |
| 10a | tert-butyl-6-[1-methyl-5-(5-methyl-1- | (45 mg, | UPLC-MS |
| | tetrahydropyran-2-yl-indazol-4-yl)-4-[1-(2- | 0.067 mmol, | (ES+, Method |
| | morpholinoethyl)pyrazol-4-yl]imidazol-2- | 25%) | 1): 1.51 min, |
| | yl]-2-azaspiro[3.3]heptane-2-carboxylate | | m/z 671.1 |
| | | | [M + H]⁺ |
| | 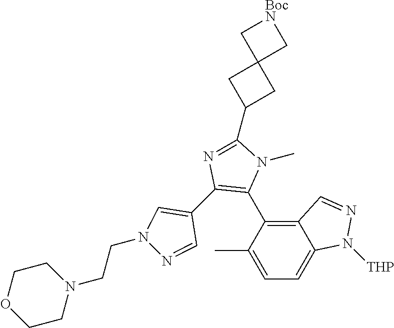 | | |
| 11a | tert-butyl-6-[4-(1,4-dimethylindazol-5-yl)- | (80 mg, | UPLC-MS |
| | 1-methyl-5-(5-methyl-1-tetrahydropyran- | 0.112 mmol, | (ES+, Method |
| | 2-yl-indazol-4-yl)imidazol-2-yl]-2- | 43% yield) | 3): 1.74 min, |
| | azaspiro[3.3]heptane-2-carboxylate | | m/z 636.4 |
| | | | [M + H]⁺ |
| | 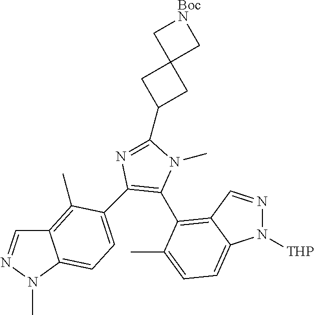 | | |
| 12a | tert-butyl-6-[4-(1,3-dimethylpyrazol-4-yl)- | (20 mg, | UPLC-MS |
| | 1-methyl-5-(5-methyl-1-tetrahydropyran- | 0.034 mmol, | (ES+, Method |
| | 2-yl-indazol-4-yl)imidazol-2-yl]-2- | 19% yield) | 3): 1.63 min, |
| | azaspiro[3.3]heptane-2-carboxylate | | m/z 586.3 |
| | | | [M + H]⁺ |
| | 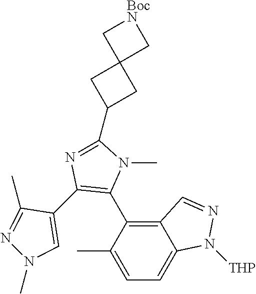 | | |
| 13a | tert-butyl-6-[4-(1-ethylpyrazol-4-yl)-1- | (50 mg, | UPLC-MS |
| | methyl-5-(5-methyl-1-tetrahydropyran-2- | 0.080 mmol, | (ES+, Method |
| | yl-indazol-4-yl)imidazol-2-yl]-2- | 49% yield) | 3): 1.80 min, |
| | azaspiro[3.3]heptane-2-carboxylate | | m/z 586.4 |
| | | | [M + H]⁺ |
| | 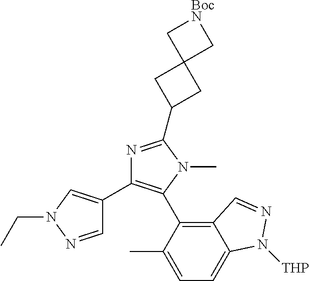 | | |
| 14a | tert-butyl-6-[1-methyl-4-(2-methylindazol- | (50 mg, | UPLC-MS |
| | 5-yl)-5-(5-methyl-1-tetrahydropyran-2-yl- | 0.080 mmol, | (ES+, Method |
| | indazol-4-yl)imidazol-2-yl]-2- | 31% yield) | 3): 1.70 min, |
| | azaspiro[3.3]heptane-2-carboxylate | | m/z 622.2 |
| | | | [M + H]⁺ |
| | 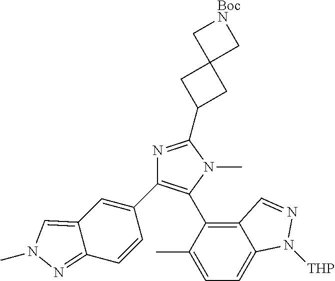 | | |
| 15a | tert-butyl-6-[4-[1-(2- | (78 mg, | UPLC-MS |
| | methoxyethyl)pyrazol-4-yl]-1-methyl-5-(5- | 0.127 mmol, | (ES+, Method |
| | methyl-1-tetrahydropyran-2-yl-indazol-4- | 48% yield) | 3): 1.62 min, |
| | yl)imidazol-2-yl]-2-azaspiro[3.3]heptane- | 616.3 | m/z 616.3 |
| | 2-carboxylate | | [M + H]⁺ |
| | 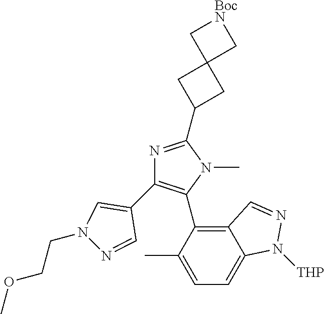 | | |
| 16a | tert-butyl-6-[4-(1,3-dimethylindazol-5-yl)- | (55 mg, | UPLC-MS |
| | 1-methyl-5-(5-methyl-1-tetrahydropyran- | 0.086 mmol, | (ES+, Method |
| | 2-yl-indazol-4-yl)imidazol-2-yl]-2- | 41% yield) | 3): 1.70 min, |
| | azaspiro[3.3]heptane-2-carboxylate | | m/z 636.3 |
| | | | [M + H]⁺ |
| | 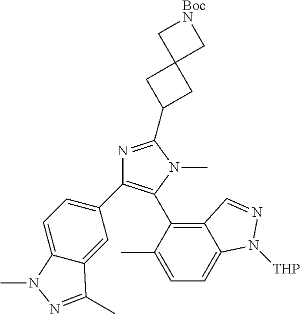 | | |
| 17a | tert-butyl-6-[4-(1,3-benzoxazol-5-yl)-1- | (50 mg, | UPLC-MS |
| | methyl-5-(5-methyl-1-tetrahydropyran-2- | 0.082 mmol, | (ES+, Method |
| | yl-indazol-4-yl)imidazol-2-yl]-2- | 39% yield) | 3): 1.65 min, |
| | azaspiro[3.3]heptane-2-carboxylate | | m/z 609.3 |
| | | | [M + H]⁺ |
| | 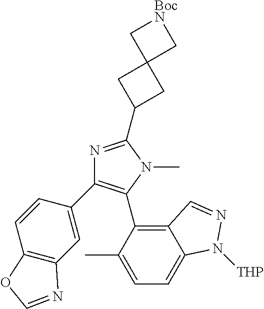 | | |
| 18a | tert-butyl-6-[1-methyl-4-(4-methyl-2,3- | (120 mg, | UPLC-MS |
| | dihydro-1,4-benzoxazin-7-yl)-5-(5- | 0.188 mmol, | (ES+, Method |
| | methyl-1-tetrahydropyran-2-yl-indazol-4- | 71% yield) | 3): 1.81 min, |
| | yl)imidazol-2-yl]-2-azaspiro[3.3]heptane- | | m/z 639.4 |
| | 2-carboxylate | | [M + H]⁺ |
| | 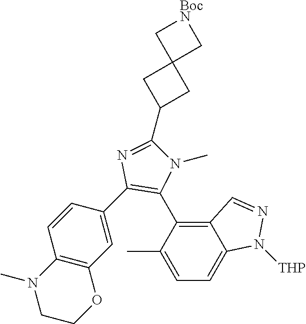 | | |
| 19a | tert-butyl 6-[4-[4-[2-(dimethylamino)-2- | (80 mg, | UPLC-MS |
| | oxo-ethoxy]phenyl]-1-methyl-5-(5-methyl- | 0.120 mmol, | (ES⁺, Method |
| | 1-tetrahydropyran-2-yl-indazol-4- | 45% yield) | 3): 1.68 min, |
| | yl)imidazol-2-yl]-2-azaspiro[3.3]heptane- | | m/z 669.3 |
| | 2-carboxylate | | [M + H]⁺ |
| | 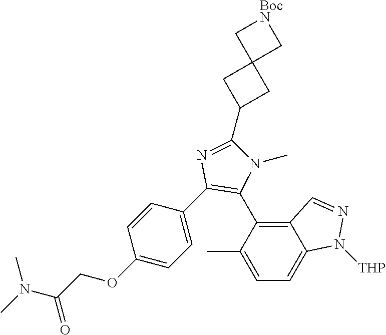 | | |
| 20a | 6-[1-methyl-5-(5-methyl-1- | (50 mg, | UPLC-MS |
| | tetrahydropyran-2-yl-indazol-4-yl)-4-[4-(2- | 0.070 mmol, | (ES+, Method |
| | morpholino-2-oxo- | 27% yield) | 3): 1.66 min, |
| | ethoxy)phenyl]imidazol-2-yl]-2- | | m/z 711.1 |
| | azaspiro[3.3]heptane-2-carboxylate | | [M + H]⁺ |
| | 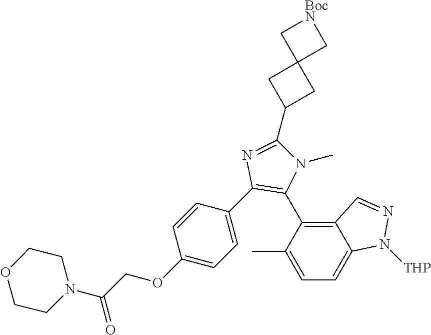 | | |
| 21a | tert-butyl 6-[4-(2-methoxythiazol-5-yl)-1- | (70 mg, | UPLC-MS |
| | methyl-5-(5-methyl-1-tetrahydropyran-2- | 0.116 mmol, | (ES⁺, Method |
| | yl-indazol-4-yl)imidazol-2-yl]-2- | 51% yield) | 3): 1.82 min, |
| | azaspiro[3.3]heptane-2-carboxylate | | m/z 605.3 |
| | | | [M + H]⁺ |
| | 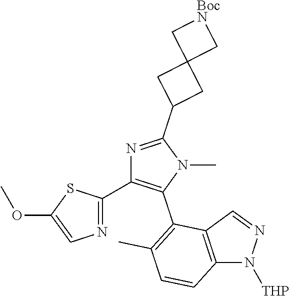 | | |
| 22a | tert-butyl 6-[4-[4-[2- | (100 mg, | UPLC-MS |
| | (dimethylamino)ethylcarbamoyl]phenyl]- | 0.147 mmol, | (ES⁺, Method |
| | 1-methyl-5-(5-methyl-1-tetrahydropyran- | 64% yield) | 3): 1.53 min, |
| | 2-yl-indazol-4-yl)imidazol-2-yl]-2- | | m/z 682.11 |
| | azaspiro[3.3]heptane-2-carboxylate | | [M + H]⁺ |
| | 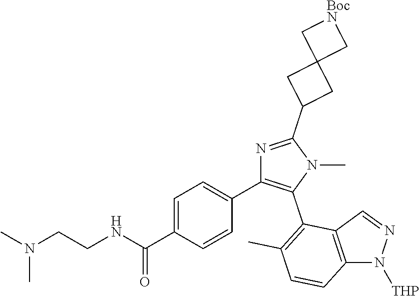 | | |
| 23a | tert-butyl 6-[4-[3-methoxy-4-(2,2,2- | (90 mg, | (ES⁺, Method |
| | trifluoroethylcarbamoyl)phenyl]-1-methyl- | 0.124 mmol, | 3): 1.86 min, |
| | 5-(5-methyl-1-tetrahydropyran-2-yl- | 55% yield) | m/z 723.3 |
| | indazol-4-yl)imidazol-2-yl]-2- | | [M + H]⁺ |
| | azaspiro[3.3]heptane-2-carboxylate | | |
| | 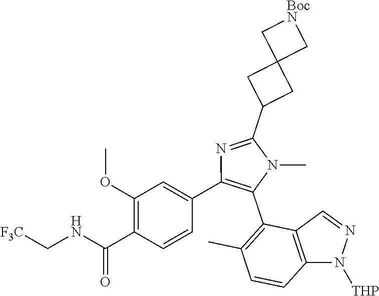 | | |
| 24a | tert-butyl 6-[1-ethyl-4-(1-methylindazol-5- | (77 mg, | UPLC-MS |
| | yl)-5-(5-methyl-1-tetrahydropyran-2-yl- | 0.121 mmol, | (ES+, Method |
| | indazol-4-yl)imidazol-2-yl]-2- | 94% yield) | 3): 1.72 min, |
| | azaspiro[3.3]heptane-2-carboxylate | | m/z 636.4 |
| | | | [M + H]⁺ |
| | 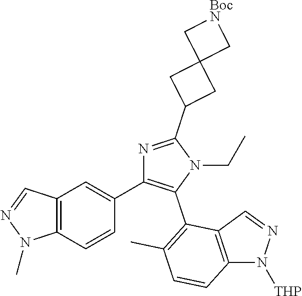 | | |

The following compounds in Table 5 were made in a similar manner to Step 2 in Example 1.

TABLE 5

| Compound | Structure | Yield | Data |
|---|---|---|---|
| 2b | 4-[2-(2-azaspiro[3.3]heptan-6-yl)-3-methyl- | (37 mg, | UPLC-MS (ES+, Method 1): 1.18 min, |
| | 5-phenyl-imidazol-4-yl]-5-methyl-1H- | 0.096 mmol, | m/z 384.3 [M + H]⁺; 192.6 |
| | indazole | 98% yield) | [M + 2H]²⁺ |
| | 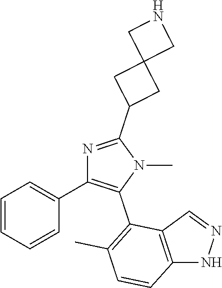 [SMILES: Cc1ccc2[nH]ncc2c1-c1c(-c2ccccc2)nc(C2CC3(CNC3)C2)n1C] | | |
| 3b | 4-[2-(2-azaspiro[3.3]heptan-6-yl)-3-methyl- | (20 mg, | UPLC-MS (ES+, Method 1): 1.18 min, |
| | 5-(3-pyridyl)imidazol-4-yl]-5-methyl-1H- | 0.052 mmol, | m/z 385.3 [M + H]⁺; 193.1 |
| | indazole | 99% yield) | [M + 2H]²⁺ |
| | 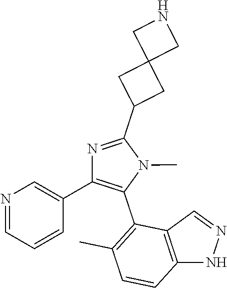 [SMILES: Cc1ccc2[nH]ncc2c1-c1c(-c2cccnc2)nc(C2CC3(CNC3)C2)n1C] | | |
| 4b | 4-[2-(2-azaspiro[3.3]heptan-6-yl)-5-(2- | (30 mg, | UPLC-MS (ES+, Method |
| | fluoro-4-methoxy-phenyl)-3-methyl- | 0.070 mmol, | 1): 1.15 min, m/z 432.2 |
| | imidazol-4-yl]-5-methyl-1H-indazole | 99% yield) | [M + H]⁺; 216.7 [M + 2H]²⁺ |
| | 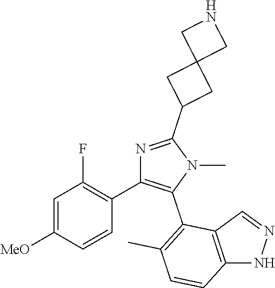 [SMILES: COc1ccc(-c2nc(C3CC4(CNC4)C3)n(C)c2-c2c(C)ccc3[nH]ncc23)c(F)c1] | | |
| 5b | 4-[4-[2-(2-azaspiro[3.3]heptan-6-yl)-1- | (80 mg, | UPLC-MS (ES+, Method 1): 1.06 min, |
| | methyl-5-(5-methyl-1H-indazol-4- | 0.17 mmol, | m/z 469.4 |
| | yl)imidazol-4-yl]phenyl]morpholine | 99% yield) | [M + H]⁺; 235.2 [M + 2H]²⁺ |
| | 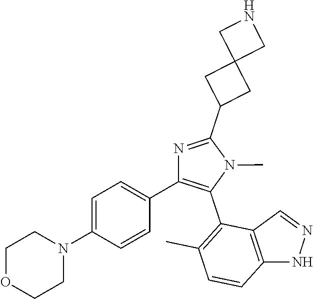 [SMILES: Cc1ccc2[nH]ncc2c1-c1c(-c2ccc(N3CCOCC3)cc2)nc(C2CC3(CNC3)C2)n1C] | | |
| 6b | give 4-[3-[2-(2-azaspiro[3.3]heptan-6-yl)-1- | (68 mg, | UPLC-MS (ES+, Method 1): 1.08 min, |
| | methyl-5-(5-methyl-1H-indazol-4- | 0.15 mmol, | m/z 469.3 [M + H]⁺; 235.2 |
| | yl)imidazol-4-yl]phenyl]morpholine | 100% yield) | [M + 2H]²⁺ |
| | 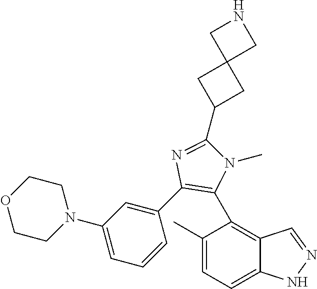 [SMILES: Cc1ccc2[nH]ncc2c1-c1c(-c2cccc(N3CCOCC3)c2)nc(C2CC3(CNC3)C2)n1C] | | |
| 7b | 4-[2-(2-azaspiro[3.3]heptan-6-yl)-3-methyl- | (80 mg, | UPLC-MS (ES+, Method 1): 1.05 min, |
| | 5-(1-methylindazol-6-yl)imidazol-4-yl]-5- | 0.18 mmol, | m/z 438.3 [M + H]⁺; 219.6 |
| | methyl-1H-indazole | 99% yield) | [M + 2H]²⁺ |
| | 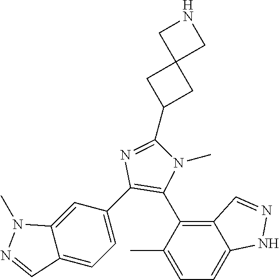 [SMILES: Cc1ccc2[nH]ncc2c1-c1c(-c2ccc3cnn(C)c3c2)nc(C2CC3(CNC3)C2)n1C] | | |
| 8b | 4-[2-(2-azaspiro[3.3]heptan-6-yl)-5-[4-(4- | (145 mg, | UPLC-MS (ES+, Method 1): 0.98 min, |
| | cyclopropylimidazol-1-yl)phenyl]-3-methyl- | 0.296 mmol, | m/z 490.3 |
| | imidazol-4-yl]-5-methyl-1H-indazole | 100% yield) | [M + H]⁺; 245.7 [M + 2H]²⁺ |
| | 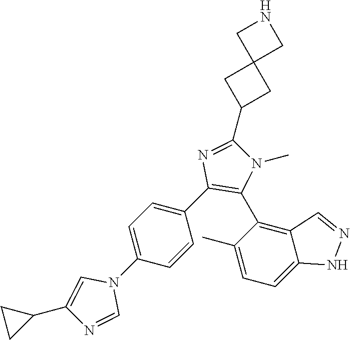 [SMILES: Cc1ccc2[nH]ncc2c1-c1c(-c2ccc(-n3cnc(C4CC4)c3)cc2)nc(C2CC3(CNC3)C2)n1C] | | |
| 9b | 5-[2-(2-azaspiro[3.3]heptan-6-yl)-1-methyl- | (50 mg, | UPLC-MS (ES+, Method 3): 0.93 min, |
| | 5-(5-methyl-1H-indazol-4-yl)imidazol-4-yl]- | 0.111 mmol, | m/z 452.2 [M + H]⁺ |
| | 1,2-dimethyl-benzimidazole | 100% yield) | |
| | 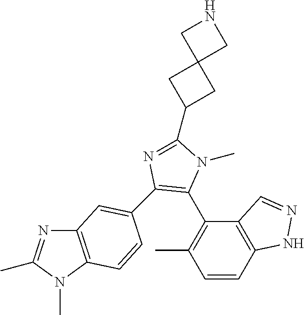 [SMILES: Cc1ccc2[nH]ncc2c1-c1c(-c2ccc3c(c2)nc(C)n3C)nc(C2CC3(CNC3)C2)n1C] | | |
| 10b | 4-[2-[4-[2-(2-azaspiro[3.3]heptan-6-yl)-1- | (55 mg, | UPLC-MS (ES+, Method |
| | methyl-5-(5-methyl-1H-indazol-4- | 0.113 mmol, | 3): 0.44 min, m/z 487.2 |
| | yl)imidazol-4-yl]pyrazol-1- | 95% yield) | [M + H]⁺ |
| | yl]ethyl]morpholine | | |
| | 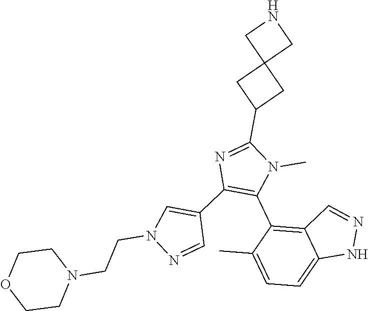 [SMILES: Cc1ccc2[nH]ncc2c1-c1c(-c2cnn(CCN3CCOCC3)c2)nc(C2CC3(CNC3)C2)n1C] | | |
| 11b | 5-[2-(2-azaspiro[3.3]heptan-6-yl)-1-methyl- | (50 mg, | UPLC-MS (ES+, Method 3): 1.15 min, |
| | 5-(5-methyl-1H-indazol-4-yl)imidazol-4-yl]- | 0.112 mmol, | m/z 452.2 |
| | 1,4-dimethyl-indazole | 88% yield) | [M + H]⁺ |
| | 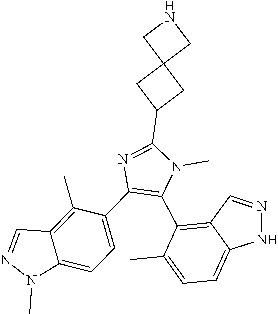 [SMILES: Cc1ccc2[nH]ncc2c1-c1c(-c2ccc3c(cnn3C)c2C)nc(C2CC3(CNC3)C2)n1C] | | |
| 12b | 4-[2-(2-azaspiro[3.3]heptan-6-yl)-5-(1,3- | (55 mg, | UPLC-MS (ES+, Method |
| | dimethylpyrazol-4-yl)-3-methyl-imidazol-4- | 0.137 mmol, | 3): 1.08 min, m/z 402.2 |
| | yl]-5-methyl-1H-indazole | 100% yield) | [M + H]⁺ |
| | 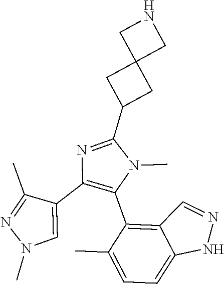 [SMILES: Cc1ccc2[nH]ncc2c1-c1c(-c2cn(C)nc2C)nc(C2CC3(CNC3)C2)n1C] | | |
| 13b | 4-[2-(2-azaspiro[3.3]heptan-6-yl)-5-(1- | (40 mg, | UPLC-MS (ES+, Method |
| | ethylpyrazol-4-yl)-3-methyl-imidazol-4-yl]- | 0.100 mmol, | 3): 1.12 min, m/z 402.2 |
| | 5-methyl-1H-indazole | 100% yield) | [M + H]⁺ |
| | 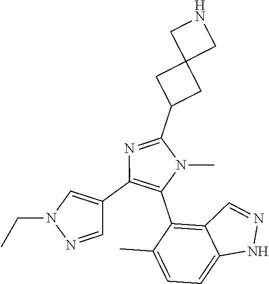 [SMILES: CCn1cc(-c2nc(C3CC4(CNC4)C3)n(C)c2-c2c(C)ccc3[nH]ncc23)cn1] | | |
| 14b | 4-[2-(2-azaspiro[3.3]heptan-6-yl)-3-methyl- | (50 mg, | UPLC-MS (ES+, Method |
| | 5-(2-methylindazol-5-yl)imidazol-4-yl]-5- | 0.114 mmol, | 3): 1.10 min, m/z 438.2 |
| | methyl-1H-indazole | 100% yield) | [M + H]⁺ |
| | 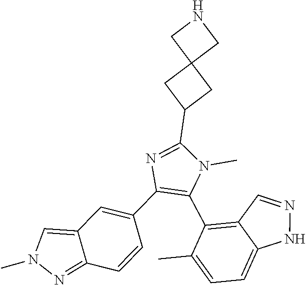 [SMILES: Cc1ccc2[nH]ncc2c1-c1c(-c2ccc3nn(C)cc3c2)nc(C2CC3(CNC3)C2)n1C] | | |
| 15b | 4-[2-(2-azaspiro[3.3]heptan-6-yl)-5-[1-(2- | (180 mg, | UPLC-MS (ES+, Method |
| | methoxyethyl)pyrazol-4-yl]-3-methyl- | 0.417 mmol, | 3): 1.02 min, m/z 432.2 |
| | imidazol-4-yl]-5-methyl-1H-indazole | 100% yield) | [M + H]⁺ |
| | 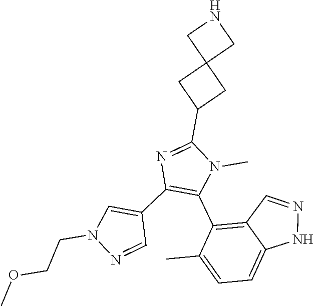 [SMILES: COCCn1cc(-c2nc(C3CC4(CNC4)C3)n(C)c2-c2c(C)ccc3[nH]ncc23)cn1] | | |
| 16b | 5-[2-(2-azaspiro[3.3]heptan-6-yl)-1-methyl- | (50 mg, | UPLC-MS (ES+, Method |
| | 5-(5-methyl-1H-indazol-4-yl)imidazol-4-yl]- | 0.11 mmol, | 3): 1.17 min, m/z 452.2 |
| | 1,3-dimethyl-indazole | 100% yield) | [M + H]⁺ |
| | 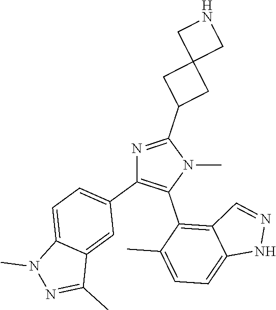 [SMILES: Cc1ccc2[nH]ncc2c1-c1c(-c2ccc3c(c2)c(C)nn3C)nc(C2CC3(CNC3)C2)n1C] | | |
| 17b | 5-[2-(2-azaspiro[3.3]heptan-6-yl)-1-methyl- | (30 mg, | UPLC-MS (ES+, Method |
| | 5-(5-methyl-1H-indazol-4-yl)imidazol-4-yl]- | 0.071 mmol, | 3): 1.10 min, m/z 425.2 |
| | 1,3-benzoxazole | 100% yield) | [M + H]⁺ |
| | 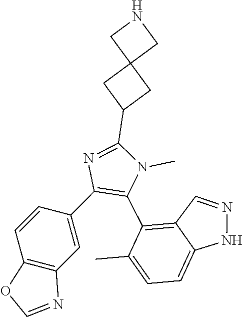 [SMILES: Cc1ccc2[nH]ncc2c1-c1c(-c2ccc3ocnc3c2)nc(C2CC3(CNC3)C2)n1C] | | |
| 18b | 7-[2-(2-azaspiro[3.3]heptan-6-yl)-1-methyl- | (85 mg, | UPLC-MS (ES+, Method |
| | 5-(5-methyl-1H-indazol-4-yl)imidazol-4-yl]- | 0.264 mmol, | 3): 1.24 min, m/z 455.1 |
| | 4-methyl-2,3-dihydro-1,4-benzoxazine | 98% yield) | [M + H]⁺ |
| | 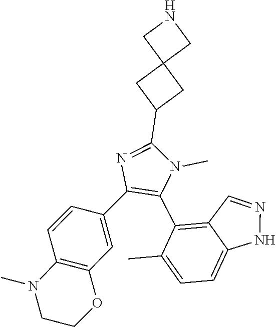 [SMILES: Cc1ccc2[nH]ncc2c1-c1c(-c2ccc3c(c2)OCCN3C)nc(C2CC3(CNC3)C2)n1C] | | |
| 19b | 2-[4-[2-(2-azaspiro[3.3]heptan-6-yl)-1- | (90 mg, | UPLC-MS (ES+, Method |
| | methyl-5-(5-methyl-1H-indazol-4- | 0.1857 mmol, | 3): 1.10 min, m/z 485.3 |
| | yl)imidazol-4-yl]phenoxy]-N,N-dimethyl-acetamide | 98.867% yield) | [M + H]⁺ |
| | 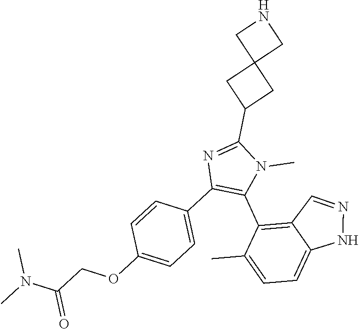 [SMILES: Cc1ccc2[nH]ncc2c1-c1c(-c2ccc(OCC(=O)N(C)C)cc2)nc(C2CC3(CNC3)C2)n1C] | | |
| 20b | 2-[4-[2-(2-azaspiro[3.3]heptan-6-yl)-1- | (50 mg, | UPLC-MS (ES+, Method |
| | methyl-5-(5-methyl-1H-indazol-4- | 0.095 mmol, | 3): 1.08 min, m/z 527.1 |
| | yl)imidazol-4-yl]phenoxy]-1-morpholino-ethanone | 72% yield) | [M + H]⁺ |
| | 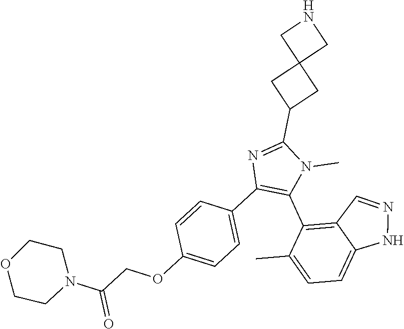 [SMILES: Cc1ccc2[nH]ncc2c1-c1c(-c2ccc(OCC(=O)N3CCOCC3)cc2)nc(C2CC3(CNC3)C2)n1C] | | |
| 21b | 5-[2-(2-azaspiro[3.3]heptan-6-yl)-1-methyl- | (35 mg, | UPLC-MS (ES⁺, Method |
| | 5-(5-methyl-1H-indazol-4-yl)imidazol-4-yl]- | 0.083 mmol, | 3): 1.12 min, m/z 421.2 [M + H]⁺ |
| | 2-methoxy-thiazole | 96% yield) | |
| | 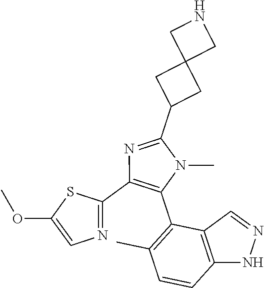 [SMILES: COc1cnc(-c2nc(C3CC4(CNC4)C3)n(C)c2-c2c(C)ccc3[nH]ncc23)s1] | | |
| 22b | 4-[2-(2-azaspiro[3.3]heptan-6-yl)-1-methyl- | (70 mg, | UPLC-MS (ES⁺, Method |
| | 5-(5-methyl-1H-indazol-4-yl)imidazol-4-yl]- | 0.141 mmol, | 3): 1.00 min, m/z 498.2 |
| | N-[2-(dimethylamino)ethyl]benzamide | 96% yield) | [M + H]⁺ |
| | 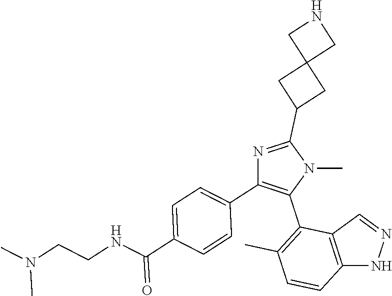 [SMILES: Cc1ccc2[nH]ncc2c1-c1c(-c2ccc(C(=O)NCCN(C)C)cc2)nc(C2CC3(CNC3)C2)n1C] | | |
| 23b | 4-[2-(2-azaspiro[3.3]heptan-6-yl)-1-methyl- | (65 mg, | UPLC-MS (ES⁺, Method |
| | 5-(5-methyl-1H-indazol-4-yl)imidazol-4-yl]- | 0.121 mmol, | 3): 1.29 min, m/z 539.2 |
| | 2-methoxy-N-(2,2,2- | 97% yield) | [M + H]⁺ |
| | trifluoroethyl)benzamide | | |
| | 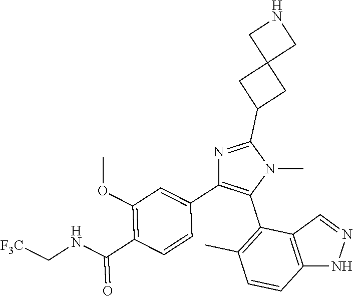 [SMILES: COc1cc(-c2nc(C3CC4(CNC4)C3)n(C)c2-c2c(C)ccc3[nH]ncc23)ccc1C(=O)NCC(F)(F)F] | | |
| 24b | 4-[2-(2-azaspiro[3.3]heptan-6-yl)-3-ethyl-5- | (54 mg, | UPLC-MS (ES+, Method |
| | (1-methylindazol-5-yl)imidazol-4-yl]-5- | 0.120 mmol, | 3): 1.26 min, m/z 452.2 [M + H]⁺ |
| | methyl-1H-indazole | 99% yield) | |
| | 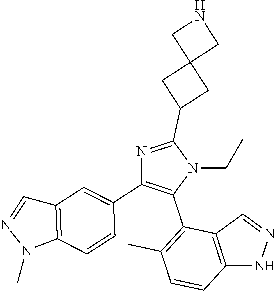 [SMILES: CCn1c(C2CC3(CNC3)C2)nc(-c2ccc3c(cnn3C)c2)c1-c1c(C)ccc2[nH]ncc12] | | |

The following examples in Table 6 were made in a similar manner to Step 3 in Example 1.

TABLE 6

| Ex- | | | |
|---|---|---|---|
| ample | Structure | Yield | Data |
| 2 | 1-[6-[1-methyl-5-(5- | (5 mg, | UPLC-MS (ES+, Method 2): 2.69 min, |
| | methyl-1H-indazol-4- | 0.0114 mmol | m/z 438.5 [M + H]⁺ |
| | yl)-4-phenyl-imidazol-2- | 12% yield) | ¹H-NMR (400 MHz, DMSO-d₆) δ/ppm: |
| | yl]-2- | | 13.16 (1H, br s), 7.59 (1H, d, J = |
| | azaspiro[3.3]heptan-2- | | 8.8 Hz), 7.53 (1H, s), 7.39 (1H, d, J = |
| | yl]prop-2-en-1-one | | 8.8 Hz), 7.32-7.28 (2H, m), 7.13-7.08 |
| | 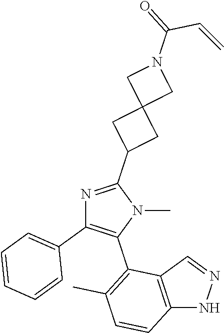 [SMILES: C=CC(=O)N1CC2(CC(c3nc(-c4ccccc4)c(-c4c(C)ccc5[nH]ncc45)n3C)C2)C1] | | (2H, m), 7.05-7.00 (1H, m), 6.37-6.28 (1H, m), 6.14-6.07 (1H, m), 5.70-5.65 (1H, m), 4.38 (1H, s), 4.24 (1H, s), 4.09 (1H, s), 3.96 (1H, s), 3.69-3.58 (1H, m), 3.10 (3H, s), 2.75-2.62 (4H, m), 2.08 (3H, s). |
| 3 | 1-[6-[1-methyl-5-(5- | (10 mg, | UPLC-MS (ES+, Method 2): 2.41 min, |
| | methyl-1H-indazol-4- | 0.023 mmol, | m/z 439.2 [M + H]⁺ |
| | yl)-4-(3- | 44% yield) | ¹H-NMR (400 MHz, DMSO-d₆) δ/ppm: |
| | pyridyl)imidazol-2-yl]-2- | | 13.23 (1H, br s), 8.37-8.34 (1H, m), |
| | azaspiro[3.3]heptan-2- | | 8.22 (1H, dd, J = 4.8 Hz, 1.6 Hz), 7.67- |
| | yl]prop-2-en-1-one | | 7.61 (2H, m), 7.55 (1H, s), 7.41 (1H, d, |
| | 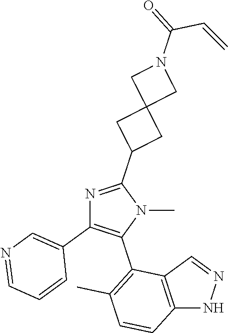 [SMILES: C=CC(=O)N1CC2(CC(c3nc(-c4cccnc4)c(-c4c(C)ccc5[nH]ncc45)n3C)C2)C1] | | J = 8.8 Hz), 7.20-7.15 (1H, m), 6.37- 6.28 (1H, m), 6.14-6.07 (1H, m), 5.70- 5.65 (1H, m), 4.38 (1H, s), 4.25 (1H, s), 4.09 (1H, s), 3.96 (1H, s), 3.71-3.60 (1H, m), 3.14 (3H, s), 2.77-2.64 (4H, m), 2.10 (3H, s). |
| 4 | 1-[6-[4-(2-fluoro-4- | (10 mg, | UPLC-MS (ES+, Method 2): 2.75 min, |
| | methoxy-phenyl)-1- | 0.021 mmol, | m/z 486.4 [M + H]⁺ |
| | methyl-5-(5-methyl-1H- | 29% yield) | ¹H-NMR (400 MHz, DMSO-d₆) δ/ppm: |
| | indazol-4-yl)imidazol-2- | | 13.03 (1H, br s), 7.50-7.45 (2H, m), |
| | yl]-2- | | 7.29-7.21 (2H, m), 6.67-6.62 (1H, m), |
| | azaspiro[3.3]heptan-2- | | 6.56 (1H, dd, J = 12.8 Hz, 2.4 Hz), 6.37- |
| | yl]prop-2-en-1-one | | 6.26 (1H, m), 6.13-6.06 (1H, m), 5.70- |
| | 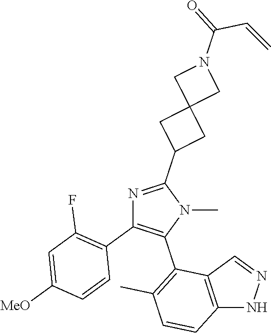 [SMILES: C=CC(=O)N1CC2(CC(c3nc(-c4ccc(OC)cc4F)c(-c4c(C)ccc5[nH]ncc45)n3C)C2)C1] | | 5.63 (1H, m), 4.37 (1H, s), 4.21 (1H, s), 4.08 (1H, s), 3.92 (1H, s), 3.68 (3H, s), 3.66-3.61 (1H, m), 3.15 (3H, s), 2.70-2.58 (4H, m), 2.05 (3H, s). |
| 5 | 1-[6-[1-methyl-5-(5- | (28 mg, | UPLC-MS (ES+, Method 2): 2.77 min, |
| | methyl-1H-indazol-4- | 0.054 mmol, | m/z 523.3 [M + H]⁺ |
| | yl)-4-(4- | 31% yield) | ¹H-NMR (400 MHz, DMSO-d₆) δ/ppm: |
| | morpholinophenyl) | | 13.14 (1H, br s), 7.57 (1H, d, J = |
| | imidazol-2-yl]-2- | | 8.8 Hz), 7.52 (1H, s), 7.37 (1H, d, J = |
| | azaspiro[3.3]heptan-2- | | 8.4 Hz), 7.16 (2H, d, J = 8.4 Hz), 6.68 |
| | yl]prop-2-en-1-one | | (2H, d, J = 8.4 Hz), 6.37-6.21 (1H, m), |
| | 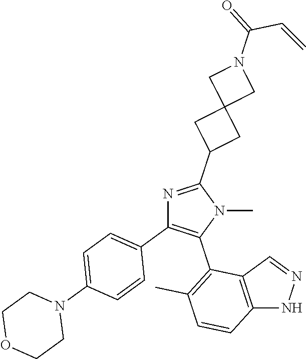 [SMILES: C=CC(=O)N1CC2(CC(c3nc(-c4ccc(N5CCOCC5)cc4)c(-c4c(C)ccc5[nH]ncc45)n3C)C2)C1] | | 6.10 (1H, ddd, J = 16.8 Hz, 4.0 Hz, 2.0 Hz), 5.70-5.64 (1H, m), 4.37 (1H, s), 4.23 (1H, s), 4.09 (1H, s), 3.94 (1H, s), 3.70-3.55 (5H, m), 3.07 (3H, s), 3.02-2.95 (4H, m), 2.74-2.60 (4H, m), 2.08 (3H, s). |
| 6 | 1-[6-[1-methyl-5-(5- | (42 mg, | UPLC-MS (ES+, Method 2): 2.41 min, |
| | methyl-1H-indazol-4- | 0.080 mmol, | m/z 439.2 [M + H]⁺ |
| | yl)-4-(3- | 55% yield) | ¹H-NMR (400 MHz, DMSO-d₆) δ/ppm: |
| | morpholinophenyl) | | 13.15 (1H, br s), 7.60 (1H, d, J = |
| | imidazol-2-yl]-2- | | 8.1 Hz), 7.53 (1H, s), 7.40-7.38 (1H, |
| | azaspiro[3.3]heptan-2- | | m), 7.01-6.95 (1H, m), 6.91-6.86 (1H, |
| | yl]prop-2-en-1-one | | m), 6.79-6.74 (1H, m), 6.64-6.59 (1H, |
| | 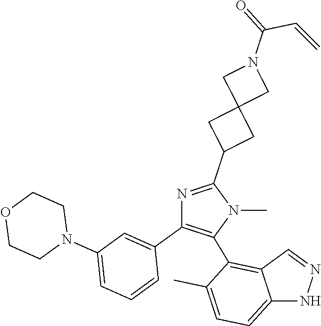 [SMILES: C=CC(=O)N1CC2(CC(c3nc(-c4cccc(N5CCOCC5)c4)c(-c4c(C)ccc5[nH]ncc45)n3C)C2)C1] | | m), 6.38-6.27 (1H, m), 6.14-6.07 (1H, m), 5.70-5.64 (1H, m), 4.37 (1H, s), 4.25 (1H, s), 4.09 (1H, s), 3.96 (1H, s), 3.67-3.55 (7H, m), 3.19-3.11 (1H, m), 2.74-2.61 (8H, m), 2.08 (3H, s). |
| 7 | 1-[6-[1-methyl-4-(1- | (40 mg, | UPLC-MS (ES+, Method 2): 2.71 min, |
| | methylindazol-6-yl)-5- | 0.08 mmol, | m/z 492.5 [M + H]⁺ |
| | (5-methyl-1H-indazol-4- | 44% yield) | ¹H-NMR (400 MHz, DMSO-d₆) δ/ppm: |
| | yl)imidazol-2-yl]-2- | | 13.18 (1H, br s), 7.85 (1H, s), 7.64- |
| | azaspiro[3.3]heptan-2- | | 7.54 (3H, m), 7.43-7.35 (2H, m), 6.95- |
| | yl]prop-2-en-1-one | | 6.91 (1H, m), 6.38-6.28 (1H, m), 6.11 |
| | 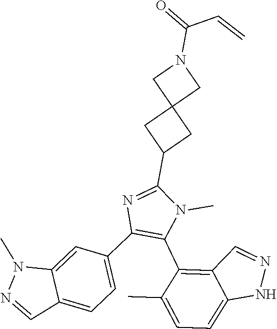 [SMILES: C=CC(=O)N1CC2(CC(c3nc(-c4ccc5cnn(C)c5c4)c(-c4c(C)ccc5[nH]ncc45)n3C)C2)C1] | | (1H, ddd, J = 17.2 Hz, 4.8 Hz, 2.4 Hz), 5.71-5.65 (1H, m), 4.40 (1H, s), 4.27 (1H, s), 4.11 (1H, s), 3.97 (1H, s), 3.83 (3H, d, J = 2.8 Hz), 3.71-3.62 (1H, m), 3.14 (3H, s), 2.80-2.66 (4H, m), 2.09 (3H, s). |
| 8 | 1-[6-[4-[4-(4- | (31 mg, | UPLC-MS (ES+, Method 2): 2.46 min, |
| | cyclopropylimidazol-1- | 0.057 mmol, | m/z 544.4 [M + H]⁺ |
| | yl)phenyl]-1-methyl-5- | 19% yield) | ¹H-NMR (400 MHz, DMSO-d₆) δ/ppm: |
| | (5-methyl-1H-indazol-4- | | 13.19 (1H, br s), 7.97-7.93 (1H, m), |
| | yl)imidazol-2-yl]-2- | | 7.65-7.51 (2H, m), 7.43-7.31 (6H, m), |
| | azaspiro[3.3]heptan-2- | | 6.38-6.27 (1H, m), 6.14-6.06 (1H, m), |
| | yl]prop-2-en-1-one | | 5.70-5.64 (1H, m), 4.38 (1H, s), 4.25 |
| | 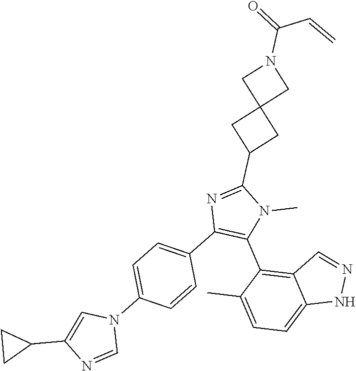 [SMILES: C=CC(=O)N1CC2(CC(c3nc(-c4ccc(-n5cnc(C6CC6)c5)cc4)c(-c4c(C)ccc5[nH]ncc45)n3C)C2)C1] | | (1H, s), 4.10 (1H, s), 3.96 (1H, s), 3.65-3.55 (1H, m), 3.11 (3H, s), 2.75- 2.60 (4H, m), 2.10 (3H, s), 1.28-1.21 (1H, m), 0.79-0.73 (2H, m), 0.67-0.61 (2H, m). |
| 9 | 1-[6-[4-(1,2- | (8 mg, | UPLC-MS (ES+, Method 2): 2.25 min, |
| | dimethylbenzimidazol- | 0.015 mmol, | m/z 506.2 [M + H]⁺ |
| | 5-yl)-1-methyl-5-(5- | 9% yield) | ¹H-NMR (400 MHz, DMSO-d₆) δ/ppm: |
| | methyl-1H-indazol-4- | | 13.15 (1H, s), 7.59 (1H, d, J = 8.5 Hz), |
| | yl)imidazol-2-yl]-2- | | 7.53 (2H, s), 7.45-7.31 (1H, m), 7.22 |
| | azaspiro[3.3]heptan-2- | | (2H, d, J = 6.6 Hz), 6.45-6.22 (1H, m), |
| | yl]prop-2-en-1-one | | 5.67 (2H, s), 4.39 (1H, s), 4.25 (1H, s), |
| | 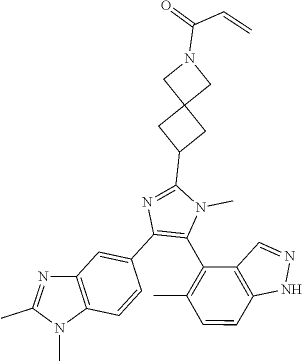 [SMILES: C=CC(=O)N1CC2(CC(c3nc(-c4ccc5c(c4)nc(C)n5C)c(-c4c(C)ccc5[nH]ncc45)n3C)C2)C1] | | 4.10 (1H, s), 3.95 (1H, d, J = 13.4 Hz), 3.62 (3H, s), 3.11 (3H, s), 2.40 (3H, s), 2.07 (3H, s), 1.24 (5H, s). |
| 10 | 1-[6-[1-methyl-5-(5- | (9 mg, | UPLC-MS (ES+, Method 2): 2.07 min, |
| | methyl-1H-indazol-4- | 0.018 mmol, | m/z 541.3 [M + H]⁺ |
| | yl)-4-[1-(2-morpho- | 11% yield) | ¹H-NMR (400 MHz, CDCl₃) δ/ppm: |
| | linoethyl)pyrazol- | | 7.63 (1H, s), 7.51 (1H, d, J = 8.5 Hz), |
| | 4-yl]imidazol-2-yl]-2- | | 7.39-7.32 (1H, m), 7.27 (1H, s), 7.15 |
| | azaspiro[3.3]heptan-2- | | (1H, d, J = 28.7 Hz), 6.41-6.29 (1H, m), |
| | yl]prop-2-en-1-one | | 6.21 (1H, ddd, J = 16.8 Hz, 10.3 Hz, |
| | 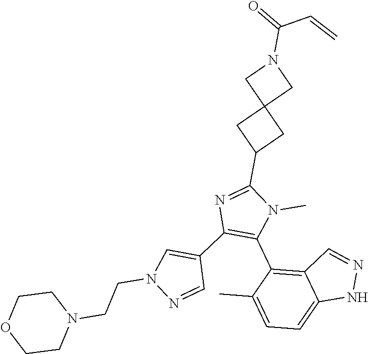 [SMILES: C=CC(=O)N1CC2(CC(c3nc(-c4cnn(CCN5CCOCC5)c4)c(-c4c(C)ccc5[nH]ncc45)n3C)C2)C1] | | 5.3 Hz), 5.68 (1H, dd, J = 10.1 Hz, 2.4 Hz), 4.37 (1H, s), 4.24 (2H, s), 4.08 (3H, dd, J = 11.4 Hz, 8.4 Hz), 3.58 (4H, t, J = 4.6 Hz,), 3.17 (3H, d, J = 4.5 Hz), 2.85 (2H, t, J = 10.7 Hz), 2.67 (5H, d, J = 8.6 Hz), 2.37 (4H, q, J = 4.1 Hz), 2.19 (3H, s). Exchangeable proton (1 NH) is not visible. |
| 11 | 1-[6-[4-(1,4- | (6 mg, | UPLC-MS (ES+, Method 2): 2.72 min, |
| | dimethylindazol-5-yl)-1- | 0.012 mmol, | m/z 506.3 [M + H]⁺ |
| | methyl-5-(5-methyl-1H- | 7% yield) | ¹H-NMR (400 MHz, CDCl₃) δ/ppm: |
| | indazol-4-yl)imidazol-2- | | 8.04 (1H, s), 7.85 (1H, d, J = 4.9 Hz), |
| | yl]-2- | | 7.66 (1H, d, J = 1.2 Hz), 7.40 (1H, d, |
| | azaspiro[3.3]heptan-2- | | J = 8.5 Hz), 7.25-7.20 (1H, m), 7.14 (1H, |
| | yl]prop-2-en-1-one | | dd, J = 8.6 Hz, 6.7 Hz), 6.99 (1H, d, J = |
| | 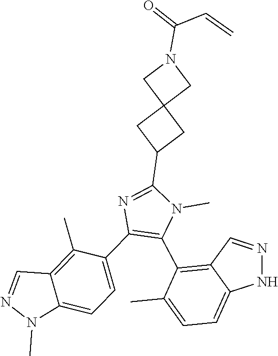 [SMILES: C=CC(=O)N1CC2(CC(c3nc(-c4ccc5c(cnn5C)c4C)c(-c4c(C)ccc5[nH]ncc45)n3C)C2)C1] | | 8.6 Hz), 6.43-6.33 (1H, m), 6.20 (1H, dt, J = 17.1 Hz, 10.3 Hz), 5.68 (1H, dt, J = 10.4 Hz, 3.0 Hz), 4.39 (1H, s), 4.25 (3H, d, J = 4.3 Hz), 4.12 (1H, d, J = 2.2 Hz), 3.96 (2H, s), 3.62 (1H, q, J = 9.0 Hz), 3.30 (2H, s), 2.98-2.86 (2H, m), 2.78-2.68 (2H, m), 2.32 (3H, d, J = 7.9 Hz), 2.08 (3H, d, J = 3.6 Hz). Exchangeable proton (1 NH) is not visible. |
| 12 | 1-[6-[4-(1,3- | (4 mg, 0.009 | UPLC-MS (ES+, Method 2): 2.47 min, |
| | dimethylpyrazol-4-yl)-1- | mmol, 12% | m/z 456.4 [M + H]⁺ |
| | methyl-5-(5-methyl-1H- | yield) | ¹H-NMR (400 MHz, CDCl₃) δ/ppm: |
| | indazol-4-yl)imidazol-2- | | 8.18 (1H, s), 7.63 (1H, d, J = 1.3 Hz), |
| | yl]-2- | | 7.56-7.44 (1H, m), 7.33 (1H, d, J = |
| | azaspiro[3.3]heptan-2- | | 8.5 Hz), 6.99 (1H, d, J = 15.1 Hz), 6.41- |
| | yl]prop-2-en-1-one | | 6.30 (1H, m,), 6.20 (1H, ddd, J = |
| | 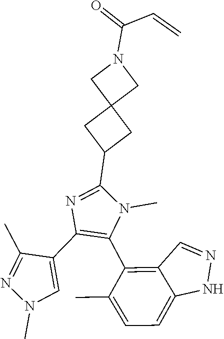 [SMILES: C=CC(=O)N1CC2(CC(c3nc(-c4cn(C)nc4C)c(-c4c(C)ccc5[nH]ncc45)n3C)C2)C1] | | 17.0 Hz, 10.2 Hz, 7.1 Hz,), 5.68 (1H, dd, J = 10.2 Hz, 2.0 Hz,), 4.37 (1H, s), 4.24 (3H, d, J = 6.9 Hz), 4.14-4.07 (1H, m), 3.66 (3H, d, J = 1.4 Hz,), 3.61-3.52 (1H, m), 3.21 (3H, d, J = 5.4 Hz), 2.94- 2.83 (1H, m), 2.74-2.65 (1H, m), 2.16 (3H, s), 1.92 (1H, s), 1.69-1.60 (1H, m), 1.30 (1H, dd, J = 14.1 Hz, 2.7 Hz,), 0.94 (1H, t, J = 7.4 Hz). |
| 13 | 1-[6-[4-(1-ethylpyrazol- | (20 mg, | UPLC-MS (ES+, Method 2): 1.17 min, |
| | 4-yl)-1-methyl-5-(5- | 0.044 mmol, | m/z 456.1 [M + H]⁺ |
| | methyl-1H-indazol-4- | 35% yield) | ¹H-NMR (400 MHz, CDCl₃) δ/ppm: |
| | yl)imidazol-2-yl]-2- | | 7.61 (1H, t, J = 1.4 Hz), 7.48 (1H, d, J = |
| | azaspiro[3.3]heptan-2- | | 8.5 Hz), 7.32 (d, J = 8.5 Hz, 1H), 7.27 |
| | yl]prop-2-en-1-one | | (s, 1H), 7.18 (1H, dd, J = 3.0 Hz, |
| | 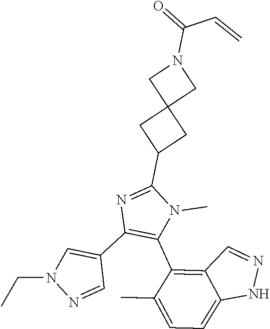 [SMILES: C=CC(=O)N1CC2(CC(c3nc(-c4cnn(CC)c4)c(-c4c(C)ccc5[nH]ncc45)n3C)C2)C1] | | 1.9 Hz), 6.36 (1H, ddd, J = 17.0 Hz, 4.4 Hz, 1.9 Hz), 6.21 (1H, ddd, J = 17.0 Hz, 10.3 Hz, 9.1 Hz), 5.69 (1H, dt, J = 10.3 Hz, 1.6 Hz), 5.51 (1H, t, J = 2.2 Hz), 4.58 (2H, tdt, J = 9.9 Hz, 6.6 Hz, 5.1 Hz), 4.36 (1H, s), 4.27 (1H, s), 4.23 (1H, s), 4.15-4.08 (1H, m), 3.56 (1H, h, J = 8.3 Hz), 3.21 (3H, d, J = 3.4 Hz), 2.81 (1H, dd, J = 11.5 Hz, 7.6 Hz), 2.77- 2.70 (2H, m), 2.13 (3H, d, J = 1.2 Hz), 1.38 (3H, t, J = 7.1 Hz), 1.30-1.22 (1H, m). |
| 14 | 1-[6-[1-methyl-4-(2- | (20 mg, | UPLC-MS (ES+, Method 2): 2.63 min, |
| | methylindazol-5-yl)-5- | 0.040 mmol, | m/z 492.3 [M + H]⁺ |
| | (5-methyl-1H-indazol-4- | 28% yield) | ¹H-NMR (400 MHz, DMSO-d₆) δ/ppm: |
| | yl)imidazol-2-yl]-2- | | 13.15 (1H, s), 8.16 (1H, s), 8.12 (1H, |
| | azaspiro[3.3]heptan-2- | | s), 7.62-7.58 (2H, m), 7.55 (1H, s), |
| | yl]prop-2-en-1-one | | 7.38 (1H, d, J = 8.6 Hz), 7.33-7.22 (1H, |
| | 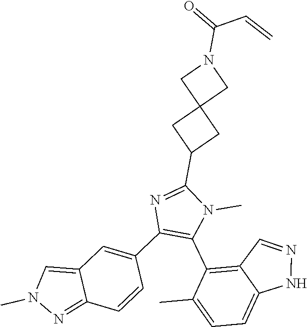 [SMILES: C=CC(=O)N1CC2(CC(c3nc(-c4ccc5nn(C)cc5c4)c(-c4c(C)ccc5[nH]ncc45)n3C)C2)C1] | | m), 7.18 (1H, d, J = 9.3 Hz), 6.33 (1H, dt, J = 17.1 Hz, 9.6 Hz), 6.19-6.05 (1H, m), 5.67 (1H, t, J = 10.1 Hz), 4.39 (1H, s), 4.25 (1H, s), 4.10 (1H, s), 4.06 (3H, s), 3.96 (1H, s), 3.10 (3H, s), 2.08 (3H, s), 1.25 (2H, s). |
| 15 | 1-[6-[4-[1-(2- | (13 mg, | UPLC-MS (ES+, Method 2): 2.46 min, |
| | methoxyethyl)pyrazol- | 0.027 mmol, | m/z 486.1 [M + H]⁺ |
| | 4-yl]-1-methyl-5-(5- | 11% yield) | ¹H-NMR (400 MHz, CDCl₃) δ/ppm: |
| | methyl-1H-indazol-4- | | 7.63 (1H, d, J = 1.1 Hz), 7.53-7.47 (1H, |
| | yl)imidazol-2-yl]-2- | | m), 7.43-7.31 (2H, m), 7.10 (1H, d, J = |
| | azaspiro[3.3]heptan-2- | | 25.1 Hz), 6.35 (1H, dt, J = 17.0 Hz, |
| | yl]prop-2-en-1-one | | 2.7 Hz,), 6.21 (1H, ddd, J = 17.0 Hz, |
| | 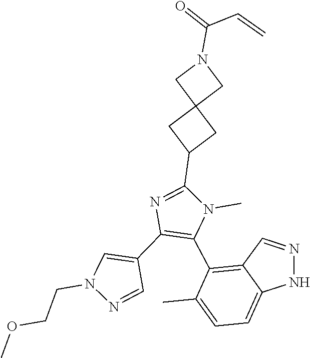 [SMILES: C=CC(=O)N1CC2(CC(c3nc(-c4cnn(CCOC)c4)c(-c4c(C)ccc5[nH]ncc45)n3C)C2)C1] | | 10.2 Hz, 4.2 Hz,), 5.73-5.59 (1H, m), 4.37 (1H, s), 4.24 (2H, d, J = 3.7 Hz), 4.12 (2H, dd, J = 6.0 Hz, 4.3 Hz,), 3.70- 3.40 (1H, m), 3.23 (3H, d, J = 1.2 Hz), 3.17 (3H, d, J = 4.8 Hz), 2.92-2.75 (2H, m), 2.73-2.55 (3H, m), 2.19 (3H, s), 1.5 (2H, s). Exchangeable proton (1 NH) is not visible. |
| 16 | 1-[6-[4-(1,3- | (14 mg, | UPLC-MS (ES+, Method 2): 2.70 min, |
| | dimethylindazol-5-yl)-1- | 0.027 mmol, | m/z 506.1 [M + H]⁺ |
| | methyl-5-(5-methyl-1H- | 15% yield) | ¹H-NMR (400 MHz, CDCl₃) δ/ppm: |
| | indazol-4-yl)imidazol-2- | | 7.81 (1H, d, J = 8.0 Hz), 7.70-7.60 (1H, |
| | yl]-2- | | m), 7.57-7.50 (1H, m), 7.38 (1H, d, J = |
| | azaspiro[3.3]heptan-2- | | 8.8 Hz), 7.27 (1H, s), 6.98 (1H, dd, J = |
| | yl]prop-2-en-1-one | | 8.8 Hz, 0.8 Hz,), 6.35 (1H, ddd, J = |
| | 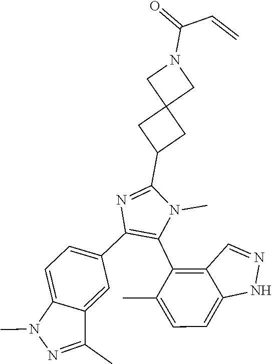 [SMILES: C=CC(=O)N1CC2(CC(c3nc(-c4ccc5c(c4)c(C)nn5C)c(-c4c(C)ccc5[nH]ncc45)n3C)C2)C1] | | 17.0 Hz, 4.3 Hz, 2.0 Hz,), 6.22 (1H, ddd, J = 16.9 Hz, 10.2 Hz, 6.0 Hz,), 5.68 (1H, ddd, J = 10.2 Hz, 4.5 Hz, 2.0 Hz), 4.40 (1H, s), 4.27 (2H, d, J = 7.5 Hz), 4.14 (1H, s), 3.88 (3H, s), 3.64-3.47 (1H, m), 3.19 (3H, d, J = 3.8 Hz), 3.00-2.85 (2H, m), 2.73 (2H, td, J = 8.1 Hz, 4.2 Hz), 2.41 (3H, d, J = 2.7 Hz,), 2.15 (3H, d, J = 1.1 Hz,). Exchangeable proton (1 NH) is not visible. |
| 17 | 1-[6-[4-(1,3- | (18 mg, | UPLC-MS (ES+, Method 2): 2.62 min, |
| | benzoxazol-5-yl)-1- | 0.036 mmol, | m/z 479.3 [M + H]⁺ |
| | methyl-5-(5-methyl-1H- | 29% yield) | ¹H-NMR (400 MHz, DMSO-d₆) δ/ppm: |
| | indazol-4-yl)imidazol-2- | | 13.20 (1H, d, J = 1.5 Hz), 8.59 (1H, s), |
| | yl]-2- | | 7.63 (1H, d, J = 8.5 Hz), 7.58-7.52 (3H, |
| | azaspiro[3.3]heptan-2- | | m), 7.47 (1H, s), 7.42 (1H, d, J = |
| | yl]prop-2-en-1-one | | 8.6 Hz), 6.33 (1H, dt, J = 17.0 Hz, |
| | 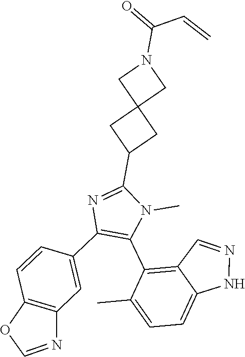 [SMILES: C=CC(=O)N1CC2(CC(c3nc(-c4ccc5ocnc5c4)c(-c4c(C)ccc5[nH]ncc45)n3C)C2)C1] | | 9.9 Hz,), 6.11 (1H, ddd, J = 16.9 Hz, 4.5 Hz, 2.3 Hz,), 5.67 (1H, ddd, J = 10.3 Hz, 7.8 Hz, 2.3 Hz,), 4.39 (1H, s), 4.25 (1H, s), 4.10 (1H, s), 3.95 (1H, d, J = 12.6 Hz), 3.68-3.56 (1H, m), 3.12 (3H, s), 2.68 (3H, d, J = 2.6 Hz), 2.33 (1H, p, J = 1.8 Hz), 2.09 (3H, s). |
| 18 | 1-[6-[1-methyl-4-(4- | (20 mg, | UPLC-MS (ES+, Method 2): 2.90 min, |
| | methyl-2,3-dihydro-1,4- | 0.039 mmol, | m/z 509.5 [M + H]⁺ |
| | benzoxazin-7-yl)-5-(5- | 15% yield) | ¹H-NMR (400 MHz, DMSO-d₆) δ/ppm: |
| | methyl-1H-indazol-4- | | 13.13 (1H, s), 7.60-7.54 (1H. m), 7.51 |
| | yl)imidazol-2-yl]-2- | | (1H, d, J = 1.4 Hz), 7.38 (1H, d, J = |
| | azaspiro[3.3]heptan-2- | | 8.6 Hz), 6.78 (1H, dt, J = 8.4 Hz, 1.7 Hz), |
| | yl]prop-2-en-1-one | | 6.58 (1H, dd, J = 3.0 Hz, 1.9 Hz), 6.45 |
| | 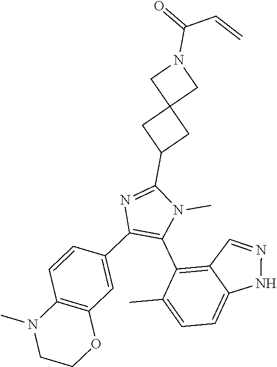 [SMILES: C=CC(=O)N1CC2(CC(c3nc(-c4ccc5c(c4)OCCN5C)c(-c4c(C)ccc5[nH]ncc45)n3C)C2)C1] | | (1H, d, J = 8.6 Hz), 6.32 (1H, dt, J = 17.0 Hz, 10.1 Hz), 6.10 (1H, ddd, J = 17.0 Hz, 4.3 Hz, 2.3 Hz), 5.67 (1H, ddd, J = 10.3 Hz, 6.8 Hz, 2.3 Hz), 4.37 (1H, s), 4.23 (1H, s), 4.09 (3H, d, J = 4.8 Hz), 3.95 (1H, s), 3.64-3.54 (1H, m), 3.11 (2H, dd, J = 5.7 Hz, 3.2 Hz), 3.05 (3H, s), 2.72 (3H, s), 2.70-2.58 (3H, m), 2.34 (1H, p, J = 1.8 Hz), 2.08 (3H, s). |
| 19 | N,N-dimethyl-2-[4-[1- | (20 mg, | UPLC-MS (ES+, Method 2): 2.61, 2.67 |
| | methyl-5-(5-methyl-1H- | 0.037 mmol, | min, m/z 539.4 [M + H]⁺ |
| | indazol-4-yl)-2-(2-prop- | 15% yield) | ¹H-NMR (400 MHz, DMSO-d₆) δ/ppm: |
| | 2-enoyl-2- | | 13.15 (1H, s), 8.20 (1H, s), 7.55 (1H, |
| | azaspiro[3.3]heptan-6- | | d, J = 19.6 Hz), 7.38 (1H, d, J = 8.4 Hz), |
| | yl)imidazol-4- | | 7.28-7.12 (2H, m), 6.66 (2H, d, J = |
| | yl]phenoxy]acetamide | | 8.8 Hz,), 6.40-6.24 (m, 1H), 6.10 (1H, |
| | 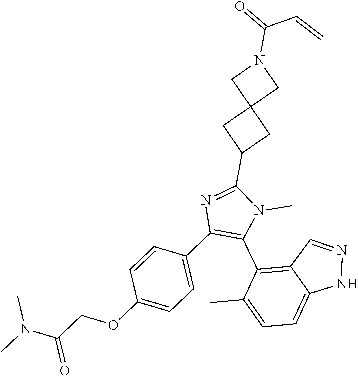 [SMILES: C=CC(=O)N1CC2(CC(c3nc(-c4ccc(OCC(=O)N(C)C)cc4)c(-c4c(C)ccc5[nH]ncc45)n3C)C2)C1] | | ddd, J = 16.9 Hz, 4.6 Hz, 2.3 Hz), 5.67 (1H, ddd, J = 10.0 Hz, 7.3 Hz, 2.3 Hz,), 4.66 (2H, s), 4.37 (1H, s), 4.23 (1H, s), 4.09 (1H, s), 3.92 (1H, d, J = 20.0 Hz), 3.69-3.55 (1H, m), 3.18 (1H, s), 3.08 (2H, s), 2.93 (3H, s), 2.80 (3H, s), 2.71-2.61 (5H, m), 2.34 (1H, p, J = 1.9 Hz), 2.08 (3H, s). NMR taken on the 4 May 2022, experiment number 15. |
| 20 | 1-[6-[1-methyl-5-(5- | (4 mg, | UPLC-MS (ES+, Long acidic, Method |
| | methyl-1H-indazol-4- | 0.007 mmol, | 2): 2.70 min, m/z 581.3 [M + H]⁺ |
| | yl)-4-[4-(2-morpholino- | 5% yield) | ¹H-NMR (400 MHz, CD₃OD) δ/ppm: |
| | 2-oxo- | | 7.60 (1H, d, J = 8.6 Hz), 7.56 (1H, s), |
| | ethoxy)phenyl]imidazol- | | 7.41 (1H, d, J = 8.6 Hz), 7.26-7.21 (2H, |
| | 2-yl]-2- | | m), 6.76 (2H, d, J = 8.9 Hz), 6.42-6.33 |
| | azaspiro[3.3]heptan-2- | | (1H, m), 6.32-6.21 (1H, m), 5.81-5.70 |
| | yl]prop-2-en-1-one | | (1H, m), 4.72 (2H, s), 4.60 (1H, s), |
| | 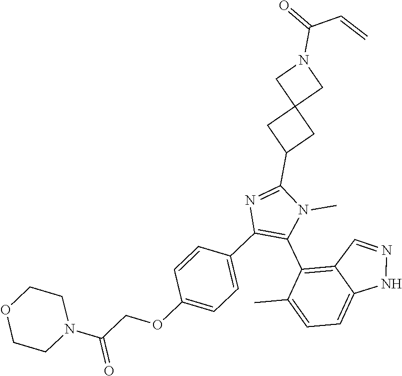 [SMILES: C=CC(=O)N1CC2(CC(c3nc(-c4ccc(OCC(=O)N5CCOCC5)cc4)c(-c4c(C)ccc5[nH]ncc45)n3C)C2)C1] | | 4.50 (1H, s), 4.34 (1H, s), 4.26 (1H, s), 4.11 (1H, s), 3.80-3.68 (1H, m), 3.65 (3H, d, J = 4.7 Hz), 3.60-3.53 (3H, m), 2.78 (1H, dd, J = 8.0 Hz, 4.0 Hz,), 2.13 (3H, s), 1.35 (4H, s), 1.31 (2H, s), 0.91 (1H, m), 1 × exchangeable proton not seen |
| 21 | 13343 | (12 mg, | UPLC-MS (ES+, Method 2): 2.60 min, |
| | 1-[6-[4-(2- | 0.026 mmol, | m/z 475.1 [M + H]⁺ |
| | methoxythiazol-5-yl)-1- | 18% yield) | ¹H NMR (400 MHz, DMSO-d₆) δ/ppm: |
| | methyl-5-(5-methyl-1H- | | 13.20 (1H, s), 7.57 (1H, d, J = 1.4 Hz), |
| | indazol-4-yl)imidazol-2- | | 7.41 (1H, d, J = 8.6 Hz), 6.43 (1H, d, |
| | yl]-2- | | J = 5.0 Hz), 6.32 (1H, dt, J = 17.1 Hz, |
| | azaspiro[3.3]heptan-2- | | 10.1 Hz,), 6.10 (1H, ddd, J = 17.0 Hz, |
| | yl]prop-2-en-1-one | | 4.3 Hz, 2.3 Hz), 5.67 (1H, ddd, J = |
| | 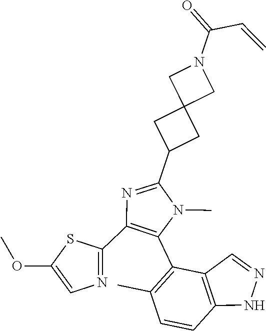 [SMILES: C=CC(=O)N1CC2(CC(c3nc(-c4ncc(OC)s4)c(-c4c(C)ccc5[nH]ncc45)n3C)C2)C1] | | 10.4 Hz, 5.7 Hz, 2.3 Hz,), 4.37 (1H, s), 4.22 (1H, s), 4.08 (1H, s), 3.93 (1H, s), 3.88 (3H, s), 3.61 (1H, td, J = 8.4 Hz, 5.7 Hz), 3.14 (3H, s), 2.68-2.59 (3H, m), 2.33 (1H, q, J = 1.9 Hz), 2.14 (3H, s). |
| 22 | 13350 | (33 mg, | UPLC-MS (ES+, Method 2): 2.07 min, |
| | N-[2- | 0.059 mmol, | m/z 552.3 [M + H]⁺ |
| | (dimethylamino)ethyl]- | 29% yield) | ¹H-NMR (400 MHz, DMSO-d₆) |
| | 4-[1-methyl-5-(5- | | δ/ppm: 13.18 (1H, s). 8.12 (1H, t, J = |
| | methyl-1H-indazol-4- | | 5.7 Hz), 7.62 (1H, d, J = 8.5 Hz), 7.56 |
| | yl)-2-(2-prop-2-enoyl-2- | | (1H, s), 7.52-7.54 (2H, m), 7.40 (1H, d, |
| | azaspiro[3.3]heptan-6- | | J = 8.6 Hz), 7.30-7.34 (2H, m), 6.24- |
| | yl)imidazol-4- | | 6.44 (1H, m), 6.10 (1H, ddd, J = |
| | yl]benzamide | | 17.0 Hz, 4.5 Hz, 2.3 Hz), 5.67 (1H, ddd, |
| | 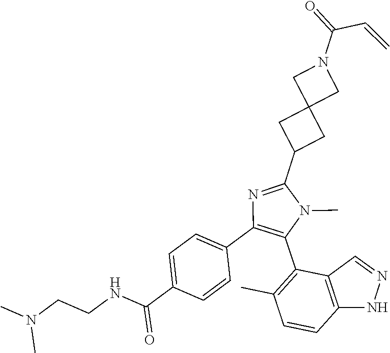 [SMILES: C=CC(=O)N1CC2(CC(c3nc(-c4ccc(C(=O)NCCN(C)C)cc4)c(-c4c(C)ccc5[nH]ncc45)n3C)C2)C1] | | J = 10.0 Hz, 7.1 Hz, 2.3 Hz), 4.38 (1H, s), 4.24 (1H, s), 4.09 (1H, s), 3.96 (1H, d, J = 10.0 Hz), 3.55-3.69 (1H, m), 3.24-3.30 (3H, m), 3.11 (3H, s), 2.67 (1H, q, J = 1.9 Hz), 2.29-2.36 (3H, m), 2.13 (7H, s), 2.08 (3H, s). |
| 23 | 13353 | (33.5 mg, | UPLC-MS (ES+, Method 2): 2.82 min, |
| | 2-methoxy-4-[1-methyl- | 0.0548 mmol, | m/z 593.3 [M + H]⁺ |
| | 5-(5-methyl-1H-indazol- | 45.433% | ¹H-NMR (400 MHz, DMSO-d₆) δ/ppm: |
| | 4-yl)-2-(2-prop-2-enoyl- | yield) | 13.20 (1H, s), 8.42 (1H, t, J = 6.4 Hz), |
| | 2-azaspiro[3.3]heptan- | | 7.65-7.54 (3H, m), 7.42 (1H, d, J = |
| | 6-yl)95midazole-4-yl]- | | 8.6 Hz), 7.14 (1H, d, J = 8.2 Hz), 6.84 |
| | N-(2,2,2-trifluoro- | | (1H, s), 6.32 (1H, dt, J = 17.1 Hz, |
| | ethyl)benzamide | | 10.3 Hz), 6.10 (1H, ddd, J = 17.1 Hz, |
| | 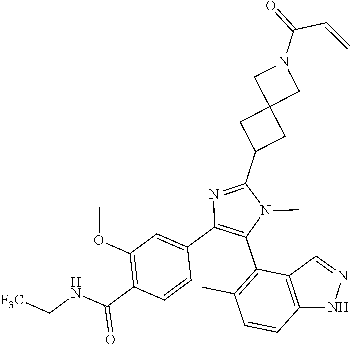 [SMILES: C=CC(=O)N1CC2(CC(c3nc(-c4ccc(C(=O)NCC(F)(F)F)c(OC)c4)c(-c4c(C)ccc5[nH]ncc45)n3C)C2)C1] | | 4.9 Hz, 2.3 Hz), 5.67 (1H, ddd, J = 9.9 Hz, 7.3 Hz, 2.3 Hz), 4.38 (1H, s), 4.25 (1H, s), 4.09 (1H, s), 4.08-3.98 (2H, m), 3.95 (1H, s), 3.70-3.59 (1H, m), 3.35 (1H, s), 3.14 (3H, s), 2.54 (1H, d, J = 2.1 Hz), 2.11 (3H, s). |
| 24 | 13363 | (3 mg, | UPLC-MS (ES+, Method 2): 2.63 min, |
| | 1-[6-[1-ethyl-4-(1- | 0.006 mmol, | m/z 506.5 [M + H]⁺ |
| | methylindazol-5-yl)-5- | 5% yield) | ¹H-NMR (400 MHz, CD₃OD) |
| | (5-methyl-1H-indazol-4- | | δ/ppm: 7.81 (1H, d, J = 0.8 Hz), 7.69- |
| | yl)imidazol-2-yl]-2- | | 7.60 (3H, m), 7.42-7.35 (2H, m), 7.31- |
| | azaspiro[3.3]heptan-2- | | 7.29 (1H, m), 6.44-.6.34 (1H, m), 6.25 |
| | yl]prop-2-en-1-one | | (1H, dt, J = 16.8 Hz, 2.0 Hz), 5.78-5.75 |
| | 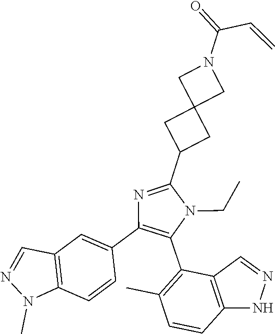 [SMILES: C=CC(=O)N1CC2(CC(c3nc(-c4ccc5c(cnn5C)c4)c(-c4c(C)ccc5[nH]ncc45)n3CC)C2)C1] | | (1H, m), 4.52 (1H, s), 4.37 (1H, s), 4.28 (1H, s), 4.15 (1H, s), 3.98 (3H, s), 3.82-3.67 (3H, m), 2.93-2.70 (4H, m), 2.12 (3H, s), 0.99 (3H, t, J = 7.2 Hz), 1 × exchangeable proton not seen. |

### Example 25—1-[6-[1-methyl-5-(1-methylindazol-5-yl)-4-(5-methyl-1H-indazol-4-yl)imidazol-2-yl]-2-azaspiro[3.3]heptan-2-yl]prop-2-en-1-one

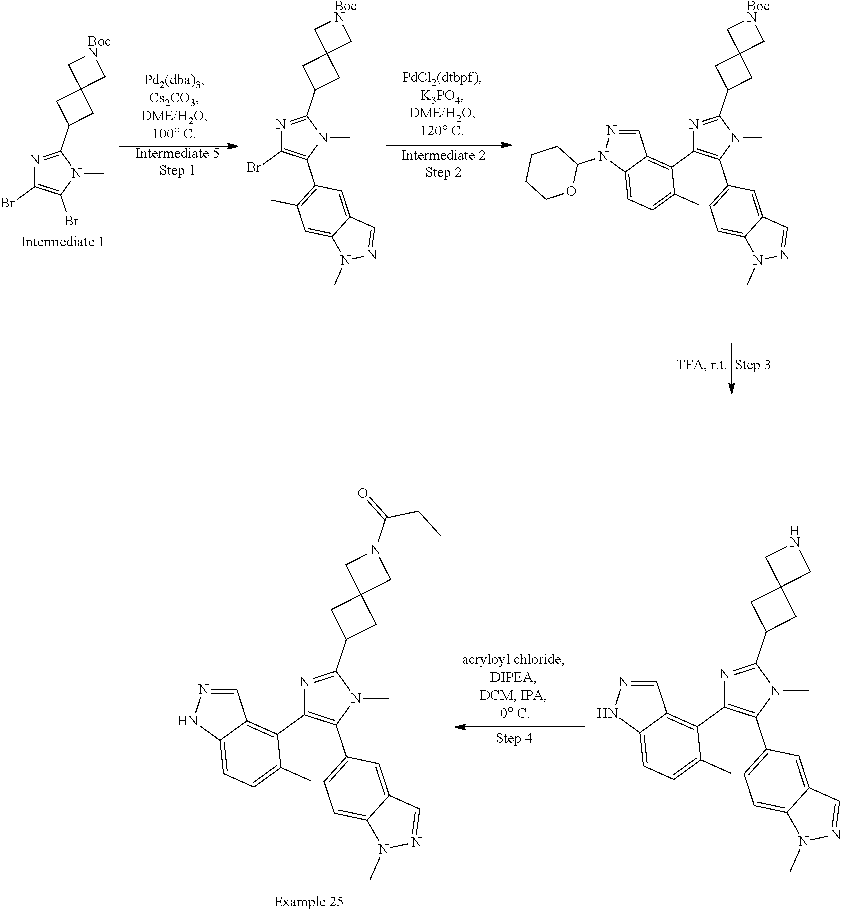 [SMILES: Cn1c(C2CC3(C2)CN(C(=O)OC(C)(C)C)C3)nc(Br)c1Br ; Cc1cc2c(cnn2C)cc1-c1c(Br)nc(C2CC3(C2)CN(C(=O)OC(C)(C)C)C3)n1C ; Cc1ccc2c(cnn2C2CCCCO2)c1-c1nc(C2CC3(C2)CN(C(=O)OC(C)(C)C)C3)n(C)c1-c1ccc2c(cnn2C)c1 ; CCC(=O)N1CC2(CC(c3nc(-c4c(C)ccc5[nH]ncc45)c(-c4ccc5c(cnn5C)c4)n3C)C2)C1 ; Cc1ccc2[nH]ncc2c1-c1nc(C2CC3(CNC3)C2)n(C)c1-c1ccc2c(cnn2C)c1]

### Step 1—tert-butyl-6-[4-bromo-1-methyl-5-(1-methylindazol-5-yl)imidazol-2-yl]-2-azaspiro[3.3]heptane-2-carboxylate

Intermediate 5 (135 mg, 0.52 mmol), (+/−)-BINAP (43 mg, 0.07 mmol), Cs₂CO₃ (1.2 g, 3.68 mmol), intermediate 1 (207 mg, 0.47 mmol), Pd₂(dba)₃ (63 mg, 0.07 mmol) in DME (8 mL) and water (1 mL) were stirred at 90° C. for 1 hour. The reaction mixture was then cooled to r.t., filtered through a celite pad, and solvent evaporated. The residue was purified using automated flash column chromatography using EtOAc in petroleum ether as the eluent system to give tert-butyl-6-[4-bromo-1-methyl-5-(1-methylindazol-5-yl)imidazol-2-yl]-2-azaspiro[3.3]heptane-2-carboxylate (40 mg, 0.08 mmol, 17% yield) as a yellow oil.

UPLC-MS (ES+, Method 3): 1.92 min, m/z 488.1 [M+H]⁺

¹H-NMR (400 MHz, CDCl₃,) δ/ppm: 8.04 (1H, d, J=1.0 Hz), 7.71 (1H, dd, J=1.5 Hz, 0.8 Hz), 7.58-7.45 (2H, m), 7.36 (1H, dd, J=8.7 Hz, 1.6 Hz), 4.12 (3H, s), 4.07 (2H, s), 3.89 (2H, s), 3.39 (3H, s), 2.80-2.68 (2H, m), 2.67-2.48 (2H, m), 1.45 (9H, s).

### Step 2—tert-butyl-6-[1-methyl-5-(1-methylindazol-5-yl)-4-(5-methyl-1-tetrahydropyran-2-yl-indazol-4-yl)imidazol-2-yl]-2-azaspiro[3.3]heptane-2-carboxylate

Intermediate 2 (28 mg, 0.08 mmol), K₃PO₄ (105 mg, 0.49 mmol), tert-butyl-6-[4-bromo-1-methyl-5-(1-methylindazol-5-yl)imidazol-2-yl]-2-azaspiro[3.3]heptane-2-carboxylate (40 mg, 0.08 mmol), PdCl₂(dtbpf) (1 mg, 0.002 mmol) in 1,4-dioxane (1.6 mL) and water (0.5 mL) were stirred at 110° C. for 1 hour. Upon formation of the product, observed via UPLCMS, crude was cooled down at room temperature, filtered through a celite pad, filtered and evaporated to give a brown oil which was purified via automated flash column chromatography using EtOAc and petroleum ether as eluent system to give tert-butyl-6-[1-methyl-5-(1-methylindazol-5-yl)-4-(5-methyl-1-tetrahydropyran-2-yl-indazol-4-yl)imidazol-2-yl]-2-azaspiro[3.3]heptane-2-carboxylate (30 mg, 0.048 mmol, 59% yield) as a yellow oil.

UPLC-MS (ES+, Method 3): 1.74 min, m/z 622.4 [M+H]⁺

¹H-NMR (400 MHz, CDCl₃) δ/ppm: 7.87 (1H, d, J=0.9 Hz), 7.84 (1H, d, J=0.9 Hz), 7.74-7.63 (1H, m), 7.47 (1H, ddd, J=7.6 Hz, 3.0 Hz, 1.1 Hz), 7.44 (1H, dd, J=1.6 Hz, 0.8 Hz), 7.33 (1H, d, J=0.9 Hz), 7.07 (2H, dd, J=8.6 Hz, 1.8 Hz), 5.65 (1H, dd, J=9.5 Hz, 2.7 Hz), 4.16-4.08 (3H, m), 4.02 (3H, s), 3.91 (2H, s), 3.77-3.70 (1H, m), 3.53 (3H, s), 2.81 (2H, td, J=9.1 Hz, 2.4 Hz,), 2.69-2.56 (2H, m), 2.05 (2H, s), 1.94 (3H, s), 1.80-1.58 (2H, m), 1.45 (9H, s), 1.26 (3H, t, J=7.1 Hz).

### Step 3—4-[2-(2-azaspiro[3.3]heptan-6-yl)-1-methyl-5-(1-methylindazol-5-yl)imidazol-4-yl]-5-methyl-1H-indazole

tert-Butyl-6-[1-methyl-5-(1-methylindazol-5-yl)-4-(5-methyl-1-tetrahydropyran-2-yl-indazol-4-yl)imidazol-2-yl]-2-azaspiro[3.3]heptane-2-carboxylate (50 mg, 0.08 mmol), TFA (0.01 mL, 0.16 mmol) were stirred at 10° C. for 5 min, then solvent was evaporated and crude 4-[2-(2-azaspiro[3.3]heptan-6-yl)-1-methyl-5-(1-methylindazol-5-yl)imidazol-4-yl]-5-methyl-1H-indazole (20 mg, 0.045 mmol, 57% yield) as a colourless oil which was used directly in the next step without further purification.

UPLC-MS (ES+, Method 3): 1.15 min, m/z 438.2 [M+H]⁺

### Step 4—1-[6-[1-methyl-5-(1-methylindazol-5-yl)-4-(5-methyl-1H-indazol-4-yl)imidazol-2-yl]-2-azaspiro[3.3]heptan-2-yl]prop-2-en-1-one—Example 25

4-[2-(2-Azaspiro[3.3]heptan-6-yl)-1-methyl-5-(1-methylindazol-5-yl)imidazol-4-yl]-5-methyl-1H-indazole (20 mg, 0.05 mmol), DIPEA (0.02 mL, 0.14 mmol) in DCM (0.7 mL) and 1-propanol (0.2 mL) were stirred at 0° C. A solution of acryloyl chloride (0.003 mL, 0.05 mmol) in DCM (0.2 mL) is added to the reaction mixture and stirred at 0° C. for 5 min. Then, upon completion of reaction, reaction was quenched by adding NH₃ in MeOH 7N (10 mL) to the crude and reaction mixture was stirred at 0° C. for 30 min. Reaction mixture was then diluted with water (10 mL) and passed through a phase separator, solvent was evaporated and crude was purified using alumina basic flash column chromatography using DCM/MeOH as the eluent system to give 1-[6-[1-methyl-5-(1-methylindazol-5-yl)-4-(5-methyl-1H-indazol-4-yl)imidazol-2-yl]-2-azaspiro[3.3]heptan-2-yl]prop-2-en-1-one (8 mg, 0.015 mmol, 32% yield) as a white powder.

UPLC-MS (ES⁺, Method 2): 1.21 min, m/z 492.5 [M+H]⁺

¹H-NMR (400 MHz, CDCl₃) δ/ppm: 7.86 (1H, dd, J=5.7 Hz, 1.0 Hz), 7.45 (1H, dq, J=2.6 Hz, 1.2 Hz, 0.7 Hz), 7.31-7.21 (3H, m), 7.09 (2H, dd, J=8.6 Hz, 1.4 Hz), 6.34-6.28 (1H, m), 6.23-6.11 (1H, m), 5.76-5.47 (1H, m), 4.23 (2H, d, J=4.4 Hz), 4.10-3.98 (3H, m), 3.57-3.52 (3H, m), 3.47 (1H, s), 2.97-2.81 (1H, m), 2.73-2.62 (2H, m), 2.17 (1H, s), 2.02 (3H, d, J=11.6 Hz).

### Example 26—1-[6-[5-(3-hydroxy-1-naphthyl)-1-methyl-4-(1-methylindazol-5-yl)imidazol-2-yl]-2-azaspiro[3.3]heptan-2-yl]prop-2-en-1-one

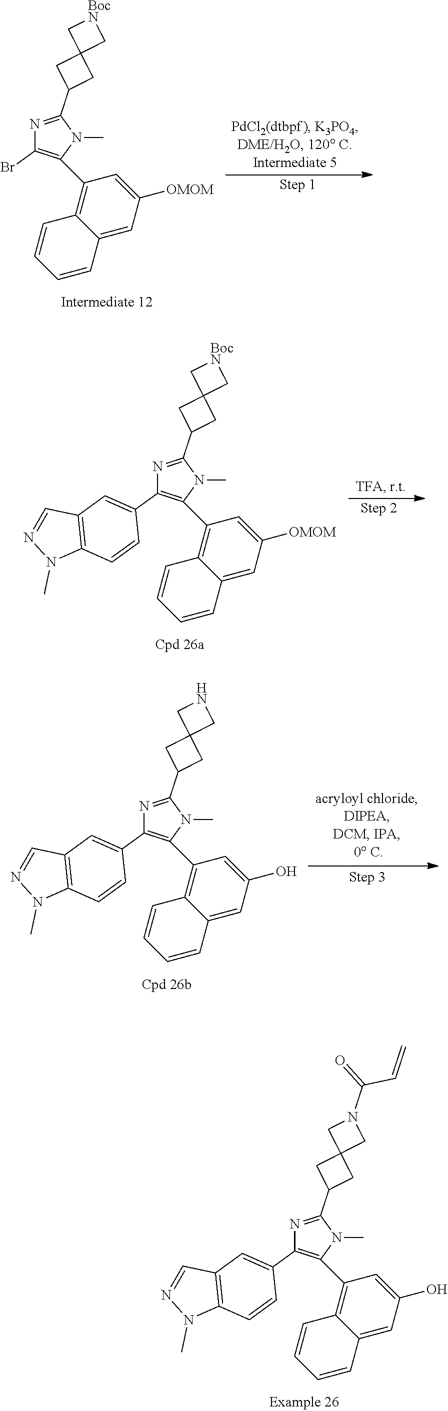

### Step 1—tert-butyl 6-[5-[3-(methoxymethoxy)-1-naphthyl]-1-methyl-4-(1-methylindazol-5-yl)imidazol-2-yl]-2-azaspiro[3.3]heptane-2-carboxylate

Intermediate 5 (200 mg, 0.77 mmol), K₃PO₄ (822 mg, 3.87 mmol), Intermediate 12 (350 mg, 0.65 mmol), PdCl₂(dtbpf) (42 mg, 0.06 mmol), 1,4-dioxane (9 mL) and water (1 mL) were combined and stirred in a sealable vial. Nitrogen was bubbled through the reaction mixture for 1 min and then the vial sealed and stirred at 100° C. for 2 hours. The reaction was then cooled to room temperature and solvent removed in vacuo. The residue was taken up in DCM (5 mL) and filtered through celite and the filter cake washed with DCM (5 mL). The filtrate was washed concentrated in vacuo and the residue purified by column chromatography using a gradient o 0-100% EtOAc in petroleum ether to give tert-butyl 6-[5-[3-(methoxymethoxy)-1-naphthyl]-1-methyl-4-(1-methylindazol-5-yl)imidazol-2-yl]-2-azaspiro[3.3]heptane-2-carboxylate (130 mg, 0.22 mmol, 34% yield) as a yellow solid.

UPLC-MS (ES+, Method 1): 1.60 min, m/z 594.7 [M+H]⁺

### Step 2—4-[2-(2-azaspiro[3.3]heptan-6-yl)-3-methyl-5-(1-methylindazol-5-yl)imidazol-4-yl]naphthalen-2-ol

tert-Butyl 6-[5-[3-(methoxymethoxy)-1-naphthyl]-1-methyl-4-(1-methylindazol-5-yl)imidazol-2-yl]-2-azaspiro[3.3]heptane-2-carboxylate (130 mg, 0.22 mmol) was stirred in TFA (0.02 mL, 0.22 mmol) at 25° C. for 5 hours. After this time the solvent was removed in vacuo and the mixture azeotroped with MeOH 3 times to give 4-[2-(2-azaspiro[3.3]heptan-6-yl)-3-methyl-5-(1-methylindazol-5-yl)imidazol-4-yl]naphthalen-2-ol (98 mg, 0.22 mmol, 99% yield) as an orange oil which was used in the next step without further purification.

UPLC-MS (ES+, Method 1): 1.22 min, m/z 450.2 [M+H]⁺; 225.6 [M+2H]²⁺

### Step 3—1-[6-[5-(3-hydroxy-1-naphthyl)-1-methyl-4-(1-methylindazol-5-yl)imidazol-2-yl]-2-azaspiro[3.3]heptan-2-yl]prop-2-en-1-one—Example 26

Acryloyl chloride (0.02 mL, 0.22 mmol) was added to a stirred solution of 4-[2-(2-azaspiro[3.3]heptan-6-yl)-3-methyl-5-(1-methylindazol-5-yl)imidazol-4-yl]naphthalen-2-ol (98 mg, 0.22 mmol), DIPEA (0.08 mL, 0.44 mmol), 1-propanol (1 mL) and DCM (3 mL) with cooling in ice and the reaction stirred for 10 min. NH₃ in MeOH (2 mL) was then added and the mixture stirred for 30 min. Water (30 mL) and DCM (30 mL) were added and the mixture was passed through a phase separator and the organic layer collected and solvent removed in vacuo. The residue was purified by column chromatography using an eluent of 0-10% MeOH in DCM to give 1-[6-[5-(3-hydroxy-1-naphthyl)-1-methyl-4-(1-methylindazol-5-yl)imidazol-2-yl]-2-azaspiro[3.3]heptan-2-yl]prop-2-en-1-one (4 mg, 0.008 mmol, 4% yield) as a white solid.

UPLC-MS (ES+, Method 2): 2.89 min, m/z 504.3 [M+H]⁺

¹H-NMR (400 MHz, CD₃OD) δ/ppm: 7.82-7.73 (3H, m), 7.45-7.36 (3H, m), 7.30-7.20 (3H, m), 7.08-7.06 (1H, m), 6.43-6.35 (1H, m), 6.30-6.23 (1H, m), 5.79-5.73 (1H, m), 4.50 (1H, s), 4.36 (1H, s), 4.27 (1H, s), 4.13 (1H, s), 3.97 (3H, s), 3.81-3.70 (1H, m), 3.24 (3H, s), 2.91-2.73 (4H, m). Exchangeable proton (1 NH) is not visible.

In a similar manner to Step 1 in Example 26; starting from Intermediates 10 and 15, the following compounds in Table 7 were made using either intermediate 10 or commercially available boronates.

TABLE 7

| Compound | Structure | Yield | Data |
|---|---|---|---|
| 27a | tert-butyl 6-[5-[3-(methoxymethoxy)-1- | (120 mg, | UPLC-MS (ES+, |
| | naphthyl]-1-methyl-4-(4- | 0.19 mmol, | Method 1): 1.62 |
| | morpholinophenyl)imidazol-2-yl]-2- | 48% yield) | min, m/z 625.7 |
| | azaspiro[3.3]heptane-2-carboxylate | | [M + H]⁺ |
| | 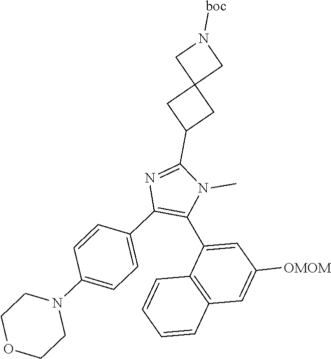 | | |
| 28a | tert-butyl 6-[5-[3-(methoxymethoxy)-1- | (70 mg, | UPLC-MS (ES+, |
| | naphthyl]-1-methyl-4-(2-methylindazol-5- | 0.118 mmol, | Method 1): 1.54 |
| | yl)imidazol-2-yl]-2-azaspiro[3.3]heptane- | 32% yield) | min, m/z 594.5 |
| | 2-carboxylate | | [M + H]⁺; 269.8 |
| | 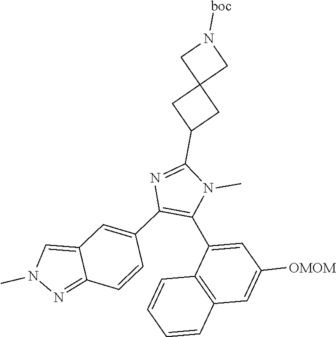 | | [M + 2H]⁺ |
| 29a | 6-[5-[3-(methoxymethoxy)-1-naphthyl]-1- | (150 mg, | UPLC-MS (ES+, |
| | methyl-4-[4-(2-morpholino-2-oxo- | 0.220 mmol, | Method 3): 1.67 |
| | ethoxy)phenyl]imidazol-2-yl]-2- | 79% yield) | min, m/z 683.3 |
| | azaspiro[3.3]heptane-2-carboxylate | | [M + H]⁺ |
| | 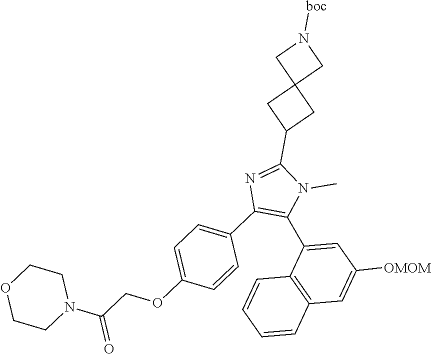 | | |
| 30a | tert-butyl 6-[5-[3-(methoxymethoxy)-1- | (58 mg, | UPLC-MS (ES⁺, |
| | naphthyl]-1-methyl-4-[4- | 0.091 mmol, | Method 3): 1.70 |
| | (morpholinomethyl)phenyl]imidazol-2-yl]- | 33% yield) | min, m/z 639.3 |
| | 2-azaspiro[3.3]heptane-2-carboxylate | | [M + H]⁺ |
| | 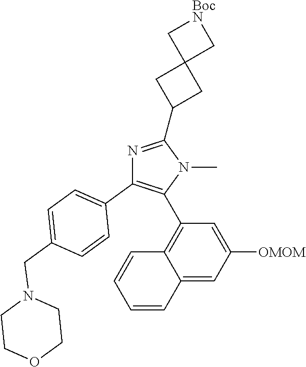 | | |
| 31a | tert-butyl 6-[5-[3-(methoxymethoxy)-1- | (150 mg, | UPLC-MS (ES⁺, |
| | naphthyl]-1-methyl-4-[4-[(4- | 0.230 mmol, | Method 3): 1.67 |
| | methylpiperazin-1- | 83% yield) | min, m/z 652.4 |
| | yl)methyl]phenyl]imidazol-2-yl]-2- | | [M + H]⁺ |
| | azaspiro[3.3]heptane-2-carboxylate | | |
| | 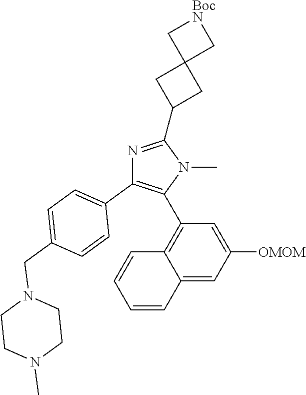 | | |
| 32a | tert-butyl 6-[1-ethyl-5-[3- | (84 mg, | UPLC-MS (ES+, |
| | (methoxymethoxy)-1-naphthyl]-4-(1- | 0.138 mmol, | Method 1): 1.61 |
| | methylindazol-5-yl)imidazol-2-yl]-2- | 38% yield)) | min, m/z 608.7 |
| | azaspiro[3.3]heptane-2-carboxylate | | [M + H]⁺ |
| | 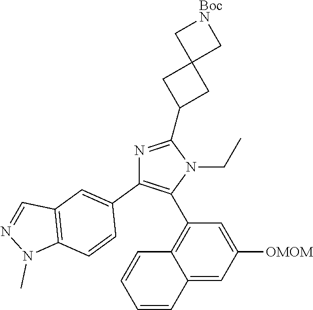 | | |
| 33a | tert-butyl 6-[1-ethyl-5-[3- | (180 mg, | UPLC-MS (ES+, |
| | (methoxymethoxy)-1-naphthyl]-4-(4- | 0.282 mmol, | Method 1): 1.68 |
| | morpholinophenyl)imidazol-2-yl]-2- | 78% yield) | min, m/z 639.7 |
| | azaspiro[3.3]heptane-2-carboxylate | | [M + H]⁺ |
| | 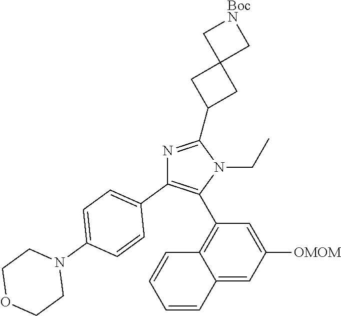 | | |

In a similar manner to Step 2 in Example 26; the following compounds in Table 8 were made.

TABLE 8

| Compounds | Structure | Yield | Data |
|---|---|---|---|
| 27b | 4-[2-(2-azaspiro[3.3]heptan-6-yl)-3- | (92 mg, | UPLC-MS (ES+, |
| | methyl-5-(4- | 0.19 mmol, | Method 1): 1.22 min, |
| | morpholinophenyl)imidazol-4- | 100% yield) | m/z 481.3 |
| | yl]naphthalen-2-ol | | [M + H]⁺; 241.1 |
| | 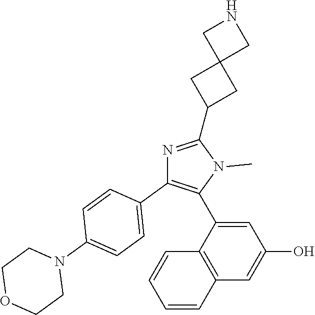 [SMILES: Cn1c(C2CC3(CNC3)C2)nc(-c2ccc(N3CCOCC3)cc2)c1-c1cc(O)cc2ccccc12] | | [M + 2H]²⁺ |
| 28b | 4-[2-(2-azaspiro[3.3]heptan-6-yl)-3- | (53 mg, | UPLC-MS (ES+, |
| | methyl-5-(2-methylindazol-5- | 0.118 mmol, | Method 1): 1.08 min, |
| | yl)imidazol-4-yl]naphthalen-2-ol | 100% yield) | m/z 450.3 [M + H]⁺; |
| | 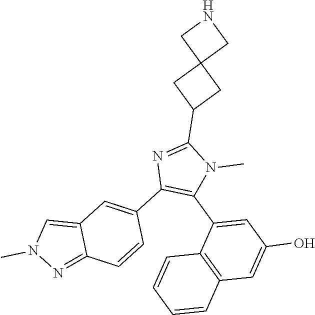 [SMILES: Cn1cc2cc(-c3nc(C4CC5(CNC5)C4)n(C)c3-c3cc(O)cc4ccccc34)ccc2n1] | | 225.7 [M + 2H]²⁺ |
| 29b | 2-[4-[2-(2-azaspiro[3.3]heptan-6-yl)- | (76 mg, | UPLC-MS (ES+, |
| | 5-(3-hydroxy-1-naphthyl)-1-methyl- | 0.141 mmol, | Method 3): 1.18 min, |
| | imidazol-4-yl]phenoxy]-1-morpholino- | 100% yield) | m/z 539.3 [M + H]⁺ |
| | ethanone | | |
| | 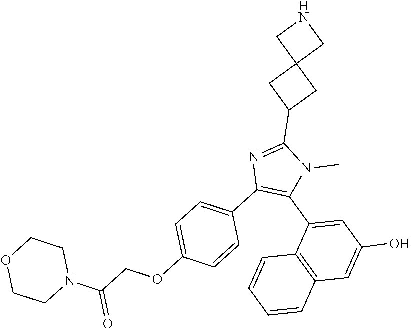 [SMILES: Cn1c(C2CC3(CNC3)C2)nc(-c2ccc(OCC(=O)N3CCOCC3)cc2)c1-c1cc(O)cc2ccccc12] | | |
| 30b | 4-[2-(2-azaspiro[3.3]heptan-6-yl)-3- | (30 mg, | UPLC-MS (ES⁺, |
| | methyl-5-[4- | 0.061 mmol, | Method 3): 1.06 min, |
| | (morpholinomethyl)phenyl]imidazol- | 77% yield) | m/z 495.4 [M + H]⁺ |
| | 4-yl]naphthalen-2-ol | | |
| | 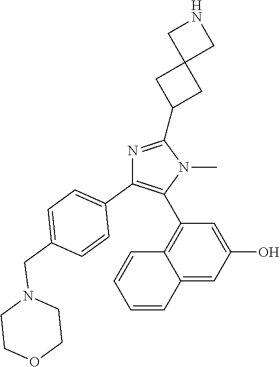 [SMILES: Cn1c(C2CC3(CNC3)C2)nc(-c2ccc(CN3CCOCC3)cc2)c1-c1cc(O)cc2ccccc12] | | |
| 31b | 4-[2-(2-azaspiro[3.3]heptan-6-yl)-3- | (45 mg, | UPLC-MS (ES⁺, |
| | methyl-5-[4-[(4-methylpiperazin-1- | 0.089 mmol, | Method 3): 1.04 min, |
| | yl)methyl]phenyl]imidazol-4- | 96% yield) | m/z 508.3 [M + H]⁺ |
| | yl]naphthalen-2-ol | | |
| | 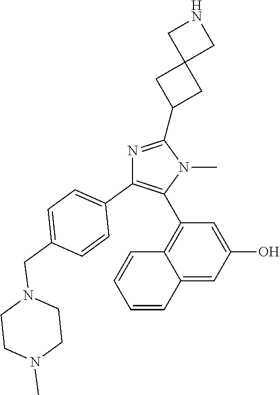 [SMILES: CN1CCN(Cc2ccc(-c3nc(C4CC5(CNC5)C4)n(C)c3-c3cc(O)cc4ccccc34)cc2)CC1] | | |
| 32b | 4-[2-(2-azaspiro[3.3]heptan-6-yl)-3- | (64 mg, | UPLC-MS (ES+, |
| | ethyl-5-(1-methylindazol-5- | 0.138 mmol, | Method 1): 1.11 min, |
| | yl)imidazol-4-yl]naphthalen-2-ol | 100% yield) | m/z 464.4 [M + H]⁺ |
| | 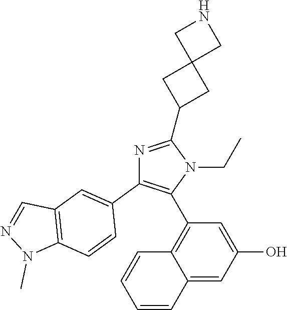 [SMILES: CCn1c(C2CC3(CNC3)C2)nc(-c2ccc3c(cnn3C)c2)c1-c1cc(O)cc2ccccc12] | | |
| 33b | 4-[2-(2-azaspiro[3.3]heptan-6-yl)-3- | (139 mg, | UPLC-MS (ES+, |
| | ethyl-5-(4- | 0.281 mmol, | Method 1): 1.15 min, |
| | morpholinophenyl)imidazol-4- | 100% yield) | m/z 495.4 [M + H]⁺ |
| | yl]naphthalen-2-ol | | |
| | 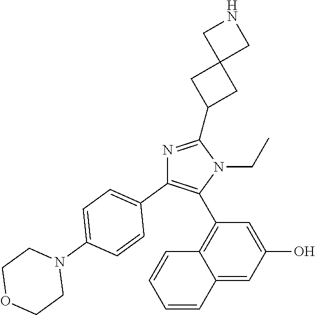 [SMILES: CCn1c(C2CC3(CNC3)C2)nc(-c2ccc(N3CCOCC3)cc2)c1-c1cc(O)cc2ccccc12] | | |

In a similar manner to Step 3 in Example 26; the following examples in Table 9 were made.

TABLE 9

| Ex- | | | |
|---|---|---|---|
| ample | Structure | Yield | Data |
| 27 | 1-[6-[5-(3-hydroxy-1-naphthyl)-1- | (25 mg, | UPLC-MS (ES+, Method |
| | methyl-4-(4- | 0.047 mmol, | 3): 2.71 min, m/z 492.5 |
| | morpholinophenyl)imidazol-2-yl]-2- | 25% yield) | [M + H]⁺ |
| | azaspiro[3.3]heptan-2-yl]prop-2-en- | | ¹H-NMR (400 MHz, DMSO- |
| | 1-one | | d₆) δ/ppm: 13.18 (1H, br s), |
| | 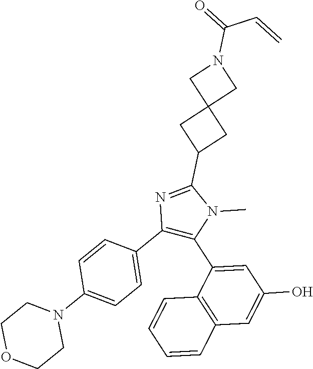 [SMILES: C=CC(=O)N1CC2(CC(c3nc(-c4ccc(N5CCOCC5)cc4)c(-c4cc(O)cc5ccccc45)n3C)C2)C1] | | 7.85 (1H, s), 7.64-7.54 (3H, m), 7.43-7.35 (2H, m), 6.95-6.91 (1H, m), 6.38- 6.28 (1H, m), 6.11 (1H, ddd, J = 17.2 Hz, 4.8 Hz, 2.4 Hz), 5.71-5.65 (1H, m), 4.40 (1H, s), 4.27 (1H, s), 4.11 (1H, s), 3.97 (1H, s), 3.83 (3H, d, J = 2.8 Hz), 3.71-3.62 (1H, m), 3.14 (3H, s), 2.80-2.66 (4H, m), 2.09 (3H, s). |
| 28 | 1-[6-[5-(3-hydroxy-1-naphthyl)-1- | (2 mg, | UPLC-MS (ES+, Method |
| | methyl-4-(2-methylindazol-5- | 0.004 mmol, | 3): 2.80 min, m/z 504.3 |
| | yl)imidazol-2-yl]-2- | 3% yield) | [M + H]⁺ |
| | azaspiro[3.3]heptan-2-yl]prop-2-en- | | ¹H-NMR (400 MHz, |
| | 1-one | | CD₃OD) δ/ppm: 7.99-7.97 |
| | 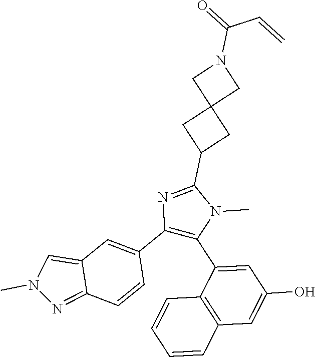 [SMILES: C=CC(=O)N1CC2(CC(c3nc(-c4ccc5nn(C)cc5c4)c(-c4cc(O)cc5ccccc45)n3C)C2)C1] | | (1H, m), 7.78-7.76 (1H, m), 7.65-7.60 (1H, m), 7.45- 7.20 (6H, m), 7.07 (1H, t, J = 2.0 Hz), 6.43-6.34 (1H, m), 6.30-6.24 (1H, m), 5.79-5.74 (1H, m), 4.50 (1H, s), 4.36 (1H, s), 4.27 (1H, s), 4.14-4.10 (4H, m), 3.80-3.70 (1H, m), 3.25- 3.23 (3H, m), 2.91-2.74 (4H, m), 1 × exchangeable OH not seen. |
| 29 | 1-[6-[5-(3-hydroxy-1-naphthyl)-1- | (30 mg, | UPLC-MS (ES+, Long |
| | methyl-4-[4-(2-morpholino-2-oxo- | 0.051 mmol, | acidic, Method 2): 2.93 min, |
| | ethoxy)phenyl]imidazol-2-yl]-2- | 36% yield) | m/z 593.3 [M + H]⁺ |
| | azaspiro[3.3]heptan-2-yl]prop-2-en- | | ¹H-NMR (400 MHz, DMSO- |
| | 1-one | | d₆) δ/ppm: 9.94 (1H, s), |
| | 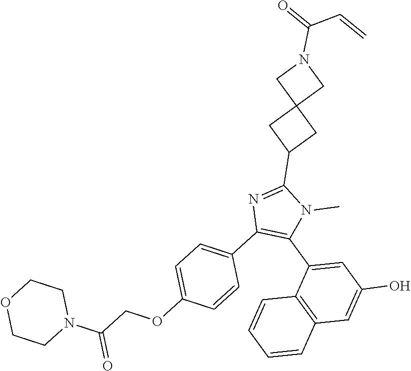 [SMILES: C=CC(=O)N1CC2(CC(c3nc(-c4ccc(OCC(=O)N5CCOCC5)cc4)c(-c4cc(O)cc5ccccc45)n3C)C2)C1] | | 7.81 (1H, d, J = 8.3 Hz), 7.50-7.40 (1H, m), 7.34- 7.22 (2H, m), 7.22-7.15 (3H, m), 7.06 (1H, d, J = 2.2 Hz), 6.67 (2H, d, J = 8.5 Hz), 6.33 (1H, ddd, J = 17.1 Hz, 10.2 Hz, 6.7 Hz), 6.15-6.04 (m, 1H), 5.74- 5.64 (1H, m), 4.68 (2H, s), 4.37 (1H, s), 4.25 (1H, s), 4.09 (1H, s), 3.96 (1H, s), 3.62 (1H, q, J = 7.7 Hz), 3.58-3.50 (4H, s), 3.43- 3.37 (5H, m), 3.07 (3H, s), 2.67 (4H, t, J = 5.6 Hz). |
| 30 | 1-[6-[5-(3-hydroxy-1-naphthyl)-1- | (18 mg, | UPLC-MS (ES⁺, Method 2): |
| | methyl-4-[4- | 0.033 mmol, | 2.25 min, m/z 549.5 [M + H]⁺ |
| | (morpholinomethyl)phenyl]imidazol- | 20% yield) | ¹H-NMR (400 MHz, DMSO- |
| | 2-yl]-2-azaspiro[3.3]heptan-2- | | d₆) δ/ppm: 9.95 (1H, s), |
| | yl]prop-2-en-1-one | | 7.81 (1H, d, J = 8.3 Hz), |
| | 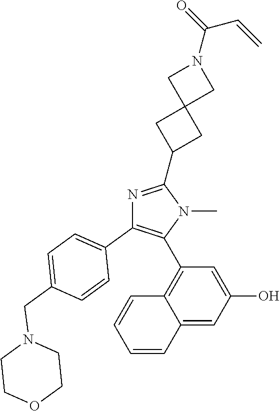 [SMILES: C=CC(=O)N1CC2(CC(c3nc(-c4ccc(CN5CCOCC5)cc4)c(-c4cc(O)cc5ccccc45)n3C)C2)C1] | | 7.43 (1H, ddd, J = 8.1 Hz, 6.2 Hz, 1.8 Hz), 7.29 (1H, d, J = 2.4 Hz), 7.27-7.18 (4H, m), 7.06 (1H, t, J = 2.0 Hz), 7.01 (2H, d, J = 8.1 Hz), 6.32 (1H, ddd, J = 17.0 Hz, 10.3 Hz, 6.6 Hz), 6.10 (1H, dt, J = 16.9 Hz, 2.9 Hz), 5.67 (1H, ddd, J = 10.3 Hz, 6.3 Hz, 2.3 Hz), 4.37 (1H, s), 4.24 (1H, s), 4.08 (1H, s), 3.95 (1H, s), 3.50 (4H, t, J = 4.6 Hz), 3.07 (3H, s), 2.71-2.65 (5H, m), 2.25 (4H, s), 1.38 (1H, d, J = 15.5 Hz), 1.19 (1H, d, J = 13.6 Hz). |
| 31 | 1-[6-[5-(3-hydroxy-1-naphthyl)-1- | (6 mg, | UPLC-MS (ES⁺, Method |
| | methyl-4-[4-[(4-methylpiperazin-1- | 0.011 mmol, | 2): 2.27 min, m/z 562.5 |
| | yl)methyl]phenyl]imidazol-2-yl]-2- | 7% yield) | [M + H]⁺ |
| | azaspiro[3.3]heptan-2-yl]prop-2-en- | | ¹H-NMR (400 MHz, DMSO- |
| | 1-one | | d₆) δ/ppm: 9.96 (1H, s), |
| | 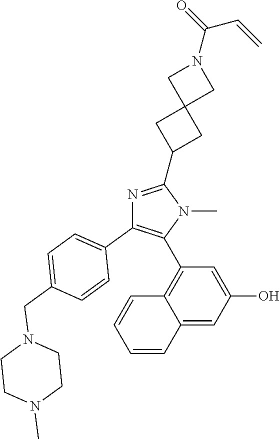 [SMILES: C=CC(=O)N1CC2(CC(c3nc(-c4ccc(CN5CCN(C)CC5)cc4)c(-c4cc(O)cc5ccccc45)n3C)C2)C1] | | 8.22 (1H, s), 7.81 (1H, d, J = 8.3 Hz), 7.43 (1H, ddd, J = 8.2 Hz, 6.3 Hz, 1.8 Hz), 7.29 (1H, d, J = 2.5 Hz), 7.24- 7.21 (3H, m), 7.10-7.05 (1H, m), 6.99 (2H, d, J = 8.1 Hz), 6.32 (1H, ddd, J = 16.8 Hz, 10.2 Hz, 6.5 Hz), 6.10 (1H, ddd, J = 17.0 Hz, 3.7 Hz, 2.3 Hz), 5.67 (1H, ddd, J = 10.2 Hz, 6.2 Hz, 2.3 Hz), 4.37 (1H, s), 4.24 (1H, s), 4.08 (1H, s), 3.95 (1H, s), 3.67-3.55 (1H, m), 3.07 (3H, s), 2.67 (4H, dt, J = 3.7 Hz, 1.9 Hz), 2.33 (2H, p, J = 1.8 Hz), 2.25 (8H, s), 2.10 (3H, s). |
| 32 | 1-[6-[1-ethyl-5-(3-hydroxy-1- | (15 mg, | UPLC-MS (ES+, Method |
| | naphthyl)-4-(1-methylindazol-5- | 0.029 mmol, | 2): 2.81 min, m/z 518.8 |
| | yl)imidazol-2-yl]-2- | 20.99% | [M + H]⁺ |
| | azaspiro[3.3]heptan-2-yl]prop-2-en- | yield) | ¹H-NMR (400 MHz, DMSO- |
| | 1-one | | d₆) δ/ppm: 9.97 (1H, |
| | 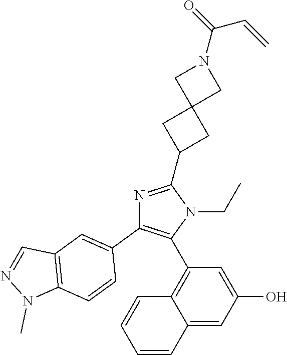 [SMILES: C=CC(=O)N1CC2(CC(c3nc(-c4ccc5c(cnn5C)c4)c(-c4cc(O)cc5ccccc45)n3CC)C2)C1] | | s), 7.84 (1H, s), 7.80 (1H, d, J = 8.0 Hz), 7.72-7.70 (1H, m), 7.43-7.38 (1H, m), 7.32-7.12 (6H, m), 6.33 (1H, ddd, J = 16.8 Hz, 10.4 Hz, 4.8 Hz), 6.14-6.07 (1H, m), 5.67 (1H, ddd, J = 10.4 Hz, 4.8 Hz, 2.4 Hz), 4.40 (1H, s), 4.27 (1H, s), 4.11 (1H, s), 3.99 (1H, s), 3.90 (3H, s), 3.71-3.59 (2H, m), 3.47-3.36 (1H, m), 2.78-2.62 (4H, m), 0.90 (3H, t, J = 7.2 Hz). |
| 33 | 1-[6-[1-ethyl-5-(3-hydroxy-1- | (15 mg, | UPLC-MS (ES Method |
| | naphthyl)-4-(4- | 0.027 mmol, | 2): 2.97 min, m/z 549.5 |
| | morpholinophenyl)imidazol-2-yl]-2- | 10% yield) | [M + H]⁺ |
| | azaspiro[3.3]heptan-2-yl]prop-2-en- | | ¹H-NMR (400 MHz, DMSO- |
| | 1-one | | d₆) δ/ppm: 9.96 (1H, |
| | 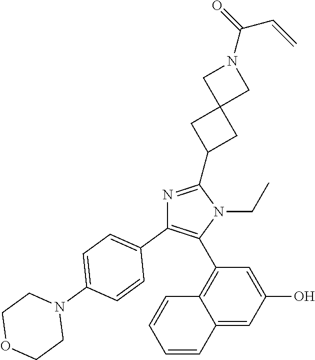 [SMILES: C=CC(=O)N1CC2(CC(c3nc(-c4ccc(N5CCOCC5)cc4)c(-c4cc(O)cc5ccccc45)n3CC)C2)C1] | | s), 7.79 (1H, d, J = 8.4 z), 7.40-7.45 (1H, m), 7.29-7.07 (6H, m), 6.66 (2H, d, J = 8.4 Hz), 6.32 (1H, ddd, J = 17.2 Hz, 6.4 Hz, 1.6 Hz), 6.09 (1H, ddd, J = 17.2 Hz, 3.6 Hz, 1.6 Hz), 5.69- 5.65 (1H, m), 4.38 (1H, s), 4.26 (1H, s), 4.09 (1H, s), 3.98 (1H, s), 3.68-3.56 (6H, m), 3.34-3.30 (1H, m), 2.99-2.94 (4H, m), 2.67- 2.57 (4H, m), 0.88 (3H, t, J = 7.2 Hz). |

### Example 34: 1-[6-[1-methyl-4-(1-methylindazol-5-yl)-5-(1-naphthyl)imidazol-2-yl]-2-azaspiro[3.3]heptan-2-yl]prop-2-en-1-one

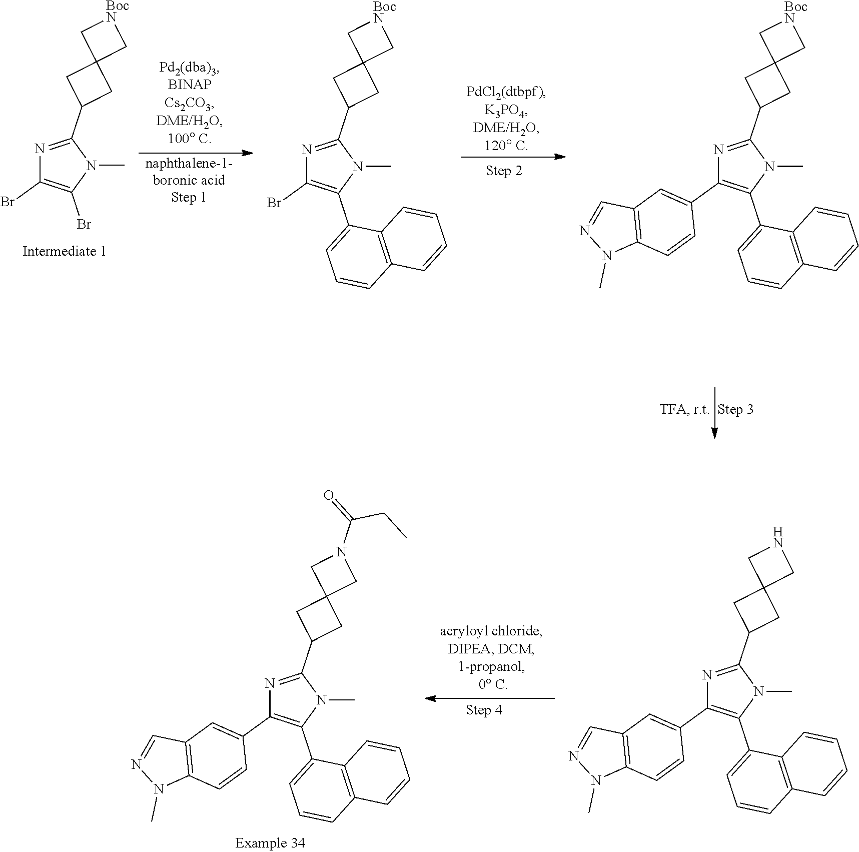 [SMILES: Cn1c(C2CC3(C2)CN(C(=O)OC(C)(C)C)C3)nc(Br)c1Br ; Cn1c(C2CC3(C2)CN(C(=O)OC(C)(C)C)C3)nc(Br)c1-c1cccc2ccccc12 ; Cn1c(C2CC3(C2)CN(C(=O)OC(C)(C)C)C3)nc(-c2ccc3c(cnn3C)c2)c1-c1cccc2ccccc12 ; CCC(=O)N1CC2(CC(c3nc(-c4ccc5c(cnn5C)c4)c(-c4cccc5ccccc45)n3C)C2)C1 ; Cn1c(C2CC3(CNC3)C2)nc(-c2ccc3c(cnn3C)c2)c1-c1cccc2ccccc12]

Step 1—tert-butyl 6-[4-bromo-1-methyl-5-(1-naphthyl)imidazol-2-yl]-2-azaspiro[3.3]heptane-2-carboxylate

(+/−)-BINAP (32.2 mg, 0.05 mmol), Intermediate 1 (150 mg, 0.34 mmol), Cs₂CO₃ (898 mg, 2.76 mmol), napthalene-1-boronic acid (71 mg, 0.41 mmol), Pd₂(dba)₃ (47 mg, 0.05 mmol), DME (3 mL) and water (1 mL) were combined in a sealable vial and stirred. The mixture was bubbled through with nitrogen for 1 minute and then the vial sealed and heated to 110° C. for 9 hours. It was then cooled to room temperature and solvent removed in vacuo. The residue was taken up in DCM (5 mL) and filtered through celite and the filter cake washed with DCM (5 mL). The filtrate was concentrated in vacuo and the residue purified by column chromatography using an eluent of 0-100% EtOAc in petroleum ether to give tert-butyl 6-[4-bromo-1-methyl-5-(1-naphthyl)imidazol-2-yl]-2-azaspiro[3.3]heptane-2-carboxylate (140 mg, 0.290 mmol, 84% yield) as a yellow solid.

UPLC-MS (ES+, Method 1): 2.04 min, m/z 482.1, 484.1 [M+H]⁺

### Step 2—tert-butyl 6-[1-methyl-4-(1-methylindazol-5-yl)-5-(1-naphthyl)imidazol-2-yl]-2-azaspiro[3.3]heptane-2-carboxylate

tert-butyl 6-[4-bromo-1-methyl-5-(1-naphthyl)imidazol-2-yl]-2-azaspiro[3.3]heptane-2-carboxylate (140 mg, 0.29 mmol), 1-methyl-5-(4,4,5,5-tetramethyl-1,3,2-dioxaborolan-2-yl)-1H-indazole (90 mg, 0.35 mmol), Pd(dppf)Cl₂·CH₂Cl₂ (19 mg, 0.03 mmol), K₃PO₄ (370 mg, 1.74 mmol), 1,4-dioxane (4.5 mL) and water (0.5 mL) were combined and stirred in a sealable vial. Nitrogen was bubbled through the reaction mixture for 1 minute and then the vial sealed and stirred at 110° C. for 1 hour. The reaction was then cooled to room temperature and solvent removed in vacuo. The residue was taken up in DCM (5 mL) and filtered through celite and the filter cake washed with DCM (5 mL). The filtrate was washed concentrated in vacuo and the residue purified by column chromatography using a gradient o 0-100% EtOAc in petroleum ether to give tert-butyl 6-[1-methyl-4-(1-methylindazol-5-yl)-5-(1-naphthyl)imidazol-2-yl]-2-azaspiro[3.3]heptane-2-carboxylate (52 mg, 0.097 mmol, 33% yield) as a yellow solid.

UPLC-MS (ES+, Method 1): 1.60 min, m/z 534.7 [M+H]⁺

### Step 3—5-[2-(2-azaspiro[3.3]heptan-6-yl)-1-methyl-5-(1-naphthyl)imidazol-4-yl]-1-methyl-indazole

tert-butyl 6-[1-methyl-4-(1-methylindazol-5-yl)-5-(1-naphthyl)imidazol-2-yl]-2-azaspiro[3.3]heptane-2-carboxylate (52 mg, 0.1 mmol) was stirred in TFA (2.0 mL, 26.12 mmol) at room temperature for 18 hours. After this time the solvent was removed in vacuo and the mixture azeotroped with MeOH 3 times to give 5-[2-(2-azaspiro[3.3]heptan-6-yl)-1-methyl-5-(1-naphthyl)imidazol-4-yl]-1-methyl-indazole (42 mg, 0.097 mmol, 99% yield) as an orange oil which was used in the next step without further purification.

UPLC-MS (ES+, Method 1): 1.28 min, m/z 434.3 [M+H]⁺; 217.7 [M+2H]²⁺

### Step 4—1-[6-[1-methyl-4-(1-methylindazol-5-yl)-5-(1-naphthyl)imidazol-2-yl]-2-azaspiro[3.3]heptan-2-yl]prop-2-en-1-one—Example 34

Acryloyl chloride (0.01 mL, 0.1 mmol) was added to a stirred solution of 5-[2-(2-azaspiro[3.3]heptan-6-yl)-1-methyl-5-(1-naphthyl)imidazol-4-yl]-1-methyl-indazole (42 mg, 0.1 mmol), DIPEA (0.05 mL, 0.29 mmol), 1-propanol (1 mL) and DCM (2 mL) with cooling in ice and the reaction stirred for 10 minutes. NH₃ in MeOH (2 mL) was then added and the mixture stirred for 30 minutes. Water (30 mL) and DCM (30 mL) were added and the mixture was passed through a phase separator and the organic layer collected and solvent removed in vacuo. The residue was purified by column chromatography using an eluent of 0-10% MeOH in DCM to give 1-[6-[1-methyl-4-(1-methylindazol-5-yl)-5-(1-naphthyl)imidazol-2-yl]-2-azaspiro[3.3]heptan-2-yl]prop-2-en-1-one (25 mg, 0.051 mmol, 53% yield) as a white solid.

UPLC-MS (ES+, Method 3): 3.14 min, m/z 488.5 [M+H]⁺

¹H-NMR (400 MHz, DMSO-d₆) δ/ppm: 8.12 (1H, d, J=8.0 Hz), 8.06 (1H, d, J=8.0 Hz), 7.83 (1H, s), 7.72-7.70 (1H, m), 7.69-7.64 (1H, m), 7.60-7.53 (2H, m), 7.47-7.45 (2H, m), 7.28 (1H, d, J=8.8 Hz), 7.23 (1H, dd, J=8.8 Hz, 1.2 Hz), 6.38-6.28 (1H, m), 6.15-6.08 (1H, m), 5.71-5.65 (1H, m), 4.40 (1H, s), 4.27 (1H, s), 4.11 (1H, s), 3.98 (1H, s), 3.90 (3H, s), 3.70-3.62 (1H, m), 3.11 (3H, s), 2.79-2.65 (4H, m).

### Example 35: 1-[6-[5-(8-chloro-3-hydroxy-1-naphthyl)-1-methyl-4-(1-methylindazol-5-yl)imidazol-2-yl]-2-azaspiro[3.3]heptan-2-yl]prop-2-en-1-one

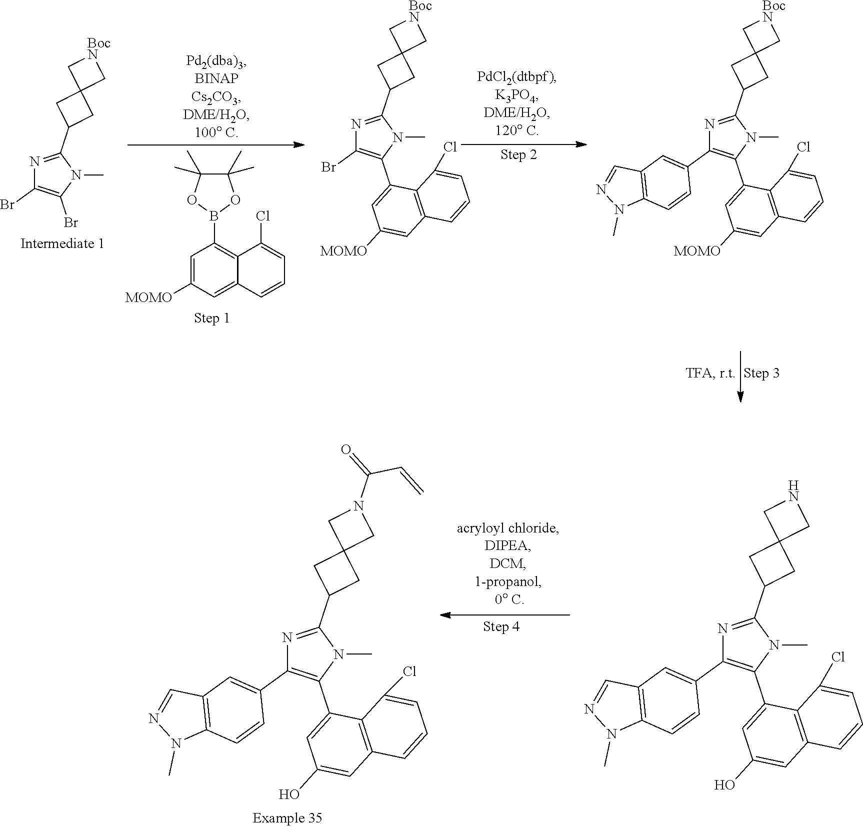

### Step 1: tert-butyl 6-[4-bromo-5-[8-chloro-3-(methoxymethoxy)-1-naphthyl]-1-methyl-imidazol-2-yl]-2-azaspiro[3.3]heptane-2-carboxylate

(+/−)-BINAP (43 mg, 0.07 mmol), 2-[8-chloro-3-(methoxymethoxy)-1-naphthyl]-4,4,5,5-tetramethyl-1,3,2-dioxaborolane (192 mg, 0.55 mmol) (WO2021/41671), Cs₂CO₃ (1.20 g, 3.68 mmol), Intermediate 1 (200 mg, 0.46 mmol), Pd₂(dba)₃ (63 mg, 0.07 mmol), DME (2 mL) and water (2 mL) were combined in a sealable vial and stirred. The mixture was bubbled through with nitrogen for 1 minute and then the vial sealed and heated to 110° C. for 6 hours. It was then cooled to room temperature and solvent removed in vacuo. The residue was taken up in DCM (5 mL) and filtered through celite and the filter cake washed with DCM (5 mL). The filtrate was concentrated in vacuo and the residue purified by column chromatography using an eluent of 0-100% EtOAc in petroleum ether to give tert-butyl 6-[4-bromo-5-[8-chloro-3-(methoxymethoxy)-1-naphthyl]-1-methyl-imidazol-2-yl]-2-azaspiro[3.3]heptane-2-carboxylate (115 mg, 0.199 mmol, 43% yield) as a yellow solid.

UPLC-MS (ES+, Method 1): 2.06 min, m/z 576.1, 578.1, 580.1 [M+H]⁺

### Step 2: tert-butyl 6-[5-[8-chloro-3-(methoxymethoxy)-1-naphthyl]-1-methyl-4-(1-methylindazol-5-yl)imidazol-2-yl]-2-azaspiro[3.3]heptane-2-carboxylate

tert-butyl 6-[4-bromo-5-[8-chloro-3-(methoxymethoxy)-1-naphthyl]-1-methyl-imidazol-2-yl]-2-azaspiro[3.3]heptane-2-carboxylate (115 mg, 0.2 mmol), 1-methyl-5-(4,4,5,5-tetramethyl-1,3,2-dioxaborolan-2-yl)-1H-indazole (62 mg, 0.24 mmol), PdCl₂(dtbpf) (13 mg, 0.02 mmol), K₃PO₄ (254 mg, 1.2 mmol), 1,4-dioxane (4.5 mL) and water (0.5 mL) were combined and stirred in a sealable vial. Nitrogen was bubbled through the reaction mixture for 1 minute and then the vial sealed and stirred at 100° C. for 3 hours. The reaction was then cooled to room temperature and solvent removed in vacuo. The residue was taken up in DCM (5 mL) and filtered through celite and the filter cake washed with DCM (5 mL). The filtrate was washed concentrated in vacuo and the residue purified by column chromatography using a gradient o 0-100% EtOAc in petroleum ether to give tert-butyl 6-[5-[8-chloro-3-(methoxymethoxy)-1-naphthyl]-1-methyl-4-(1-methylindazol-5-yl)imidazol-2-yl]-2-azaspiro[3.3]heptane-2-carboxylate (100 mg, 0.1592 mmol, 80% yield) as a yellow solid.

UPLC-MS (ES+, Method 1): 1.60 min, m/z 628.4, 630.4 [M+H]⁺

### Step 3: 4-[2-(2-azaspiro[3.3]heptan-6-yl)-3-methyl-5-(1-methylindazol-5-yl)imidazol-4-yl]-5-chloro-naphthalen-2-ol

tert-butyl 6-[5-[8-chloro-3-(methoxymethoxy)-1-naphthyl]-1-methyl-4-(1-methylindazol-5-yl)imidazol-2-yl]-2-azaspiro[3.3]heptane-2-carboxylate (100 mg, 0.16 mmol) was stirred in TFA (3.0 mL, 39.18 mmol) at room temperature for 5 hours. After this time the solvent was removed in vacuo and the mixture azeotroped with MeOH 3 times to give 4-[2-(2-azaspiro[3.3]heptan-6-yl)-3-methyl-5-(1-methylindazol-5-yl)imidazol-4-yl]-5-chloro-naphthalen-2-ol (77 mg, 0.159 mmol, 100% yield) as an orange oil which was used in the next step without further purification.

UPLC-MS (ES+, Method 3): 1.35 min, m/z 484.1, 486.1 [M+H]⁺

### Step 4: 1-[6-[5-(8-chloro-3-hydroxy-1-naphthyl)-1-methyl-4-(1-methylindazol-5-yl)imidazol-2-yl]-2-azaspiro[3.3]heptan-2-yl]prop-2-en-1-one—Example 35

Acryloyl chloride (0.01 mL, 0.16 mmol) was added to a stirred solution of 4-[2-(2-azaspiro[3.3]heptan-6-yl)-3-methyl-5-(1-methylindazol-5-yl)imidazol-4-yl]-5-chloro-naphthalen-2-ol (77 mg, 0.16 mmol), DIPEA (0.08 mL, 0.48 mmol), 1-propanol (1 mL) and DCM (3 mL) with cooling in ice and the reaction stirred for 3 hours. NH₃ in MeOH (2 mL) was then added and the mixture stirred for 30 minutes. Water (30 mL) and DCM (30 mL) were added and the mixture was passed through a phase separator and the organic layer collected and solvent removed in vacuo. The residue was purified by column chromatography using an eluent of 0-10% MeOH in DCM to give 1-[6-[5-(8-chloro-3-hydroxy-1-naphthyl)-1-methyl-4-(1-methylindazol-5-yl)imidazol-2-yl]-2-azaspiro[3.3]heptan-2-yl]prop-2-en-1-one (3 mg, 0.006 mmol, 3% yield) as a white solid.

UPLC-MS (ES+, Method 3): 2.96 min, m/z 538.3, 540.3 [M+H]⁺

¹H-NMR (400 MHz, CD₃OD) δ/ppm: 7.82 (1H, d, J=0.8 Hz), 7.78 (1H, dd, J=7.6 Hz, 2.0 Hz), 7.69 (1H, d, J=6.8 Hz), 7.43-7.25 (5H, m), 7.07 (1H, t, J=2.8 Hz), 6.43-6.33 (1H, m), 6.29-6.23 (1H, m), 5.75 (1H, ddd, J=10.4 Hz, 4.0 Hz, 2.0 Hz), 4.49 (1H, s), 4.37-4.30 (1H, m), 4.26 (1H, s), 4.15-4.08 (1H, m), 3.98 (3H, s), 3.77-3.68 (1H, m), 3.19 (3H, s), 2.91-2.71 (4H, m), 1× exchangeable proton not seen.

### Example 36—2-amino-4-[3-methyl-5-(1-methylindazol-5-yl)-2-(2-prop-2-enoyl-2-azaspiro[3.3]heptan-6-yl)imidazol-4-yl]benzothiophene-3-carbonitrile

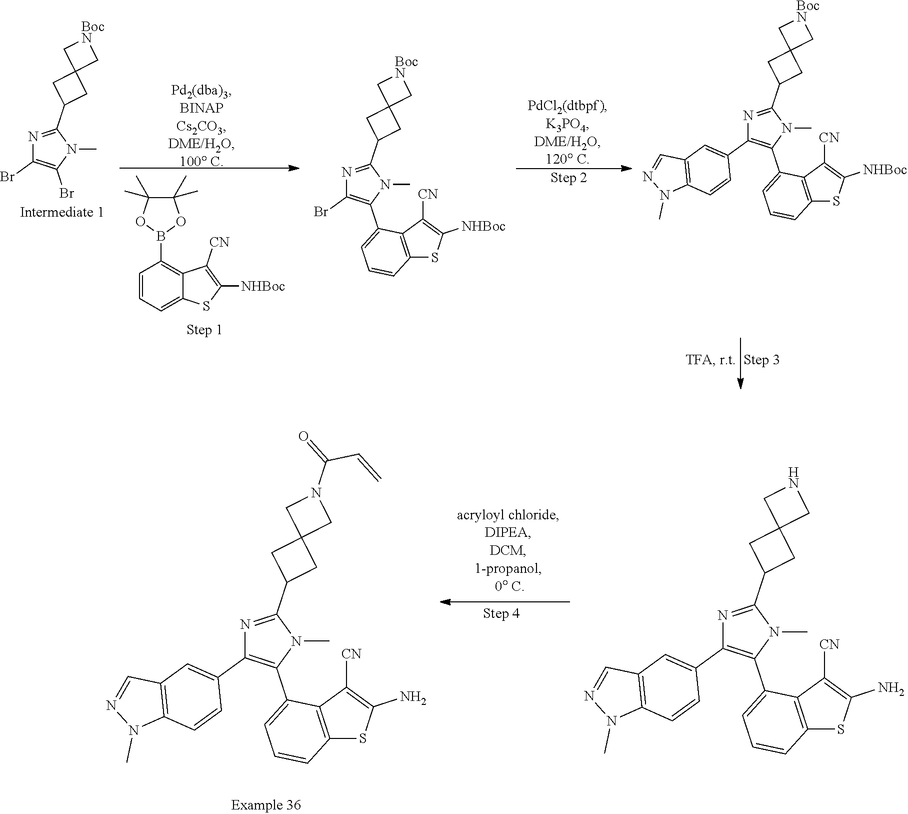 [SMILES: Cn1c(C2CC3(C2)CN(C(=O)OC(C)(C)C)C3)nc(Br)c1Br ; CC(C)(C)OC(=O)Nc1sc2cccc(B3OC(C)(C)C(C)(C)O3)c2c1C#N ; Cn1c(C2CC3(C2)CN(C(=O)OC(C)(C)C)C3)nc(Br)c1-c1cccc2sc(NC(=O)OC(C)(C)C)c(C#N)c12 ; Cn1c(C2CC3(C2)CN(C(=O)OC(C)(C)C)C3)nc(-c2ccc3c(cnn3C)c2)c1-c1cccc2sc(NC(=O)OC(C)(C)C)c(C#N)c12 ; C=CC(=O)N1CC2(CC(c3nc(-c4ccc5c(cnn5C)c4)c(-c4cccc5sc(N)c(C#N)c45)n3C)C2)C1 ; Cn1c(C2CC3(CNC3)C2)nc(-c2ccc3c(cnn3C)c2)c1-c1cccc2sc(N)c(C#N)c12]

Step 1—tert-butyl 6-[4-bromo-5-[2-(tert-butoxycarbonylamino)-3-cyano-benzothiophen-4-yl]-1-methyl-imidazol-2-yl]-2-azaspiro[3.3]heptane-2-carboxylate

Intermediate 1 (200 mg, 0.46 mmol), K₃PO₄ (146 mg, 0.69 mmol), tert-butyl N-[3-cyano-4-(4,4,5,5-tetramethyl-1,3,2-dioxaborolan-2-yl)benzothiophen-2-yl]carbamate (221 mg, 0.55 mmol) (WO2021/118877), Pd(dppf)Cl₂·CH₂Cl₂ (30 mg, 0.05 mmol) in 1,4-dioxane (3.72 mL) and water (0.87 mL) are stirred for 24 hours at 90° C. Upon completion of the reaction, reaction mixture was cooled down at room temperature, filtrate though a celite pad, evaporated and purified via automated flash column chromatography using a slow gradient of DCM/MeOH (60 CV from 0% to 100% DCM/MeOH 20%) to give tert-butyl 6-[4-bromo-5-[2-(tert-butoxycarbonylamino)-3-cyano-benzothiophen-4-yl]-1-methyl-imidazol-2-yl]-2-azaspiro[3.3]heptane-2-carboxylate (60 mg, 0.095 mmol, 21% yield) as a yellow powder.

UPLC-MS (ES+, Method 3): 2.15 min, m/z 630.2 [M+H]⁺

### Step 2—tert-butyl-6-[5-[2-(tert-butoxycarbonylamino)-3-cyano-benzothiophen-4-yl]-1-methyl-4-(1-methylindazol-5-yl)imidazol-2-yl]-2-azaspiro[3.3]heptane-2-carboxylate

1-Methyl-5-(4,4,5,5-tetramethyl-1,3,2-dioxaborolan-2-yl)-1H-indazole (37 mg, 0.14 mmol), K₃PO₄ (81 mg, 0.38 mmol), tert-butyl-6-[4-bromo-5-[2-(tert-butoxycarbonylamino)-3-cyano-benzothiophen-4-yl]-1-methyl-imidazol-2-yl]-2-azaspiro[3.3]heptane-2-carboxylate (60 mg, 0.1 mmol), PdCl₂(dtbpf) (2 mg, 0.002 mmol) in 1,4-dioxane (1.8 mL) and water (0.5 mL) were stirred at 110° C. for 1 hour. Upon formation of the product, observed via UPLCMS, crude was cooled to room temperature, filtered through a celite pad, filtered and evaporated to give a brown oil which was purified via automated flash column chromatography using DCM and MeOH (very slow gradient, 22 cv from 0 to 20% DCM/MeOH) to give tert-butyl-6-[5-[2-(tert-butoxycarbonylamino)-3-cyano-benzothiophen-4-yl]-1-methyl-4-(1-methylindazol-5-yl)imidazol-2-yl]-2-azaspiro[3.3]heptane-2-carboxylate (35 mg, 0.051 mmol, 54% yield) as a yellow oil.

UPLC-MS (ES+, Method 1): 1.81 min, m/z 680.3 [M+H]⁺

### Step 3—2-amino-4-[2-(2-azaspiro[3.3]heptan-6-yl)-3-methyl-5-(1-methylindazol-5-yl)imidazol-4-yl]benzothiophene-3-carbonitrile

tert-butyl-6-[5-[2-(tert-butoxycarbonylamino)-3-cyano-benzothiophen-4-yl]-1-methyl-4-(1-methylindazol-5-yl)imidazol-2-yl]-2-azaspiro[3.3]heptane-2-carboxylate (30 mg, 0.04 mmol) and TFA (0.45 mL, 5.85 mmol) were stirred at ˜10° C. (ice bath from previous reaction which was almost everything water) for 2 hours. Then, solvent was evaporated and crude 2-amino-4-[2-(2-azaspiro[3.3]heptan-6-yl)-3-methyl-5-(1-methylindazol-5-yl)imidazol-4-yl]benzothiophene-3-carbonitrile (30 mg, 0.062 mmol, 100% yield) as a yellow oil which was used directly in the next step

UPLC-MS (ES+, Method 3): 1.19 min, m/z 480.2 [M+H]⁺

### Step 4—2-amino-4-[3-methyl-5-(1-methylindazol-5-yl)-2-(2-prop-2-enoyl-2-azaspiro[3.3]heptan-6-yl)imidazol-4-yl]benzothiophene-3-carbonitrile—Example 36

2-amino-4-[2-(2-azaspiro[3.3]heptan-6-yl)-3-methyl-5-(1-methylindazol-5-yl)imidazol-4-yl]benzothiophene-3-carbonitrile (20 mg, 0.04 mmol), DIPEA (0.002 mL, 0.01 mmol) in DCM (0.6 mL) and 1-propanol (0.2 mL) were stirred at 0° C. A solution of acryloyl chloride (0.003 mL, 0.04 mmol) in DCM (0.2 mL) is added to the reaction mixture and stirred at 0° C. for 5 min. Then, upon completion of reaction, reaction was quenched by adding ammonia in MeOH 7N (10 mL) to the crude and reaction mixture was stirred at 0° C. for 30 min. Reaction mixture was then diluted with water (10 mL) and passed through a phase separator, solvent was evaporated and crude was purified using flash column chromatography using DCM/MeOH as eluent system to give 2-amino-4-[3-methyl-5-(1-methylindazol-5-yl)-2-(2-prop-2-enoyl-2-azaspiro[3.3]heptan-6-yl)imidazol-4-yl]benzothiophene-3-carbonitrile (14 mg, 0.025 mmol, 60% yield) as a white powder.

UPLC-MS (ES+, Method 2): 2.78 min, m/z 534.4 [M+H]⁺

¹H-NMR (400 MHz, DMSO-d₆) δ/ppm: 7.80-7.78 (1H, m), 7.75 (1H, d, J=8.3 Hz), 7.66 (1H, dd, J=7.7 Hz, 1.4 Hz), 7.59-7.51 (1H, m), 7.36-7.29 (2H, m), 7.17 (1H, d, J=8.8 Hz), 6.39-6.27 (1H, m), 6.26-6.16 (1H, m), 5.70-5.61 (1H, m), 5.20 (1H, s), 5.13 (1H, s), 4.37-4.29 (2H, m), 4.25 (1H, d, J=9.4 Hz), 4.20 (1H, d, J=3.4 Hz), 3.98 (3H, d, J=2.3 Hz), 3.53 (1H, q, J=7.6 Hz), 3.22 (3H, s), 2.81-2.65 (2H, m), 2× exchangeable NH not seen.

### Example 37—4-[5-(3-hydroxy-1-naphthyl)-1-methyl-2-(2-prop-2-enoyl-2-azaspiro[3.3]heptan-6-yl)imidazol-4-yl]-N-(2-morpholinoethyl)benzamide

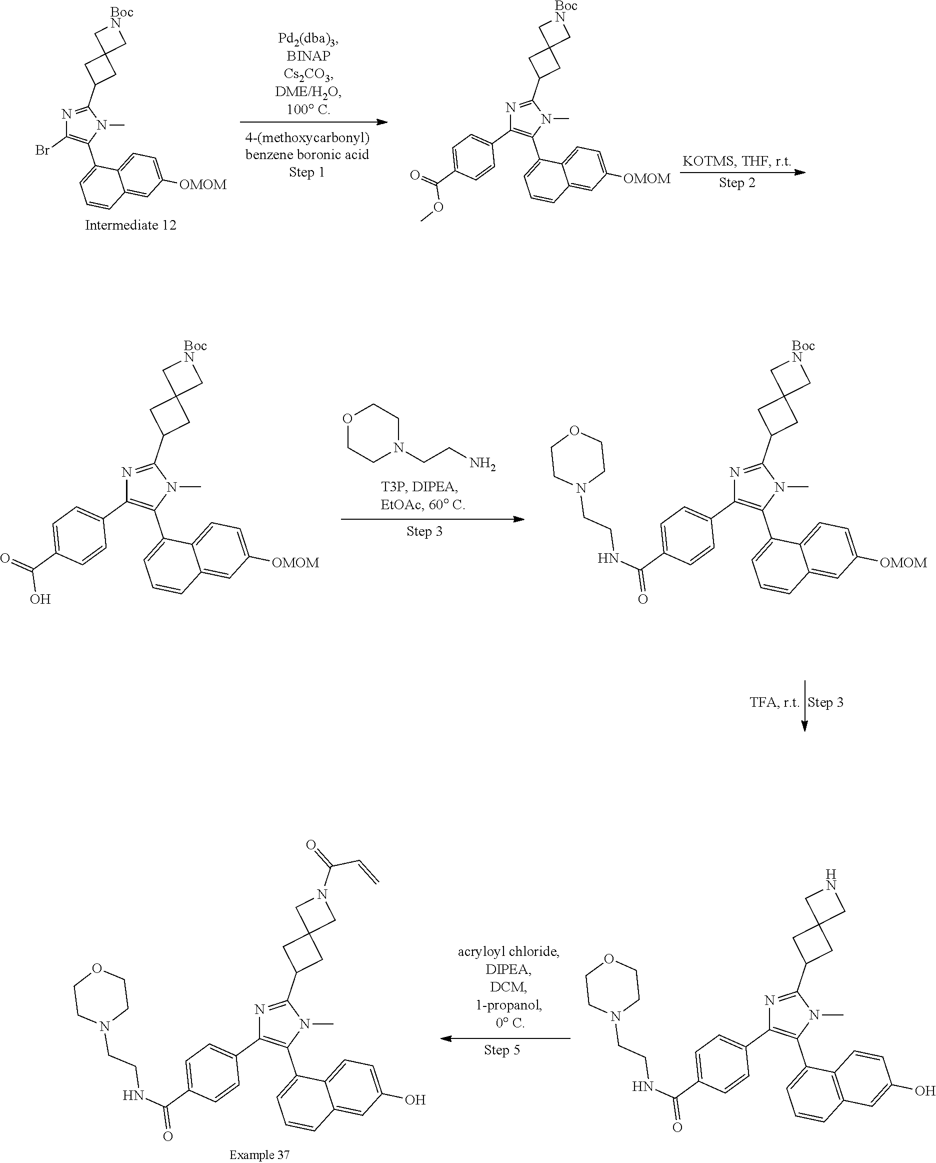

### Step 1—tert-butyl 6-[4-(4-methoxycarbonylphenyl)-5-[3-(methoxymethoxy)-1-naphthyl]-1-methyl-imidazol-2-yl]-2-azaspiro[3.3]heptane-2-carboxylate

Intermediate 5 (605 mg, 1.12 mmol), 4-(methoxycarbonyl)benzeneboronic acid (241 mg, 1.34 mmol), PdCl₂(dtbpf) (73 mg, 0.11 mmol), K₃PO₄ (1.42 g, 6.69 mmol), 1,4-dioxane (4.5 mL) and water (0.5 mL) were combined and stirred in a sealable vial. Nitrogen was bubbled through the reaction mixture for 1 minute and then the vial sealed and stirred at 110° C. for 1 hour. The reaction was then cooled to room temperature and solvent removed in vacuo. The residue was taken up in DCM (5 mL) and filtered through celite and the filter cake washed with DCM (5 mL). The filtrate was washed concentrated in vacuo and the residue purified by column chromatography using a gradient of 0-100% EtOAc in petroleum ether to give tert-butyl 6-[4-(4-methoxycarbonylphenyl)-5-[3-(methoxymethoxy)-1-naphthyl]-1-methyl-imidazol-2-yl]-2-azaspiro[3.3]heptane-2-carboxylate (235 mg, 0.393 mmol, 35% yield) as a yellow solid.

UPLC-MS (ES+, Method 1): 1.71 min, m/z 598.6 [M+H]⁺

### Step 2—4-[2-(2-tert-butoxycarbonyl-2-azaspiro[3.3]heptan-6-yl)-5-[3-(methoxymethoxy)-1-naphthyl]-1-methyl-imidazol-4-yl]benzoic acid

Potassium trimethylsilanolate (100 mg, 0.79 mmol) was added to a stirred solution of tert-butyl 6-[4-(4-methoxycarbonylphenyl)-5-[3-(methoxymethoxy)-1-naphthyl]-1-methyl-imidazol-2-yl]-2-azaspiro[3.3]heptane-2-carboxylate (235 mg, 0.39 mmol) and THE (5 mL) at room temperature under a nitrogen atmosphere. The reaction was stirred for 18 hours and then filtered and the filtrate concentrated in vacuo to give 4-[2-(2-tert-butoxycarbonyl-2-azaspiro[3.3]heptan-6-yl)-5-[3-(methoxymethoxy)-1-naphthyl]-1-methyl-imidazol-4-yl]benzoic acid (200 mg, 0.343 mmol, 87% yield) as a white solid which was used in the next step without further purification.

UPLC-MS (ES+, Method 1): 1.61 min, m/z 584.5 [M+H]⁺

### Step 3—tert-butyl 6-[5-[3-(methoxymethoxy)-1-naphthyl]-1-methyl-4-[4-(2-morpholinoethylcarbamoyl)phenyl]imidazol-2-yl]-2-azaspiro[3.3]heptane-2-carboxylate

T3P (0.25 mL, 0.51 mmol) (50% in EtOAc) was added to a stirred solution of 4-(2-Aminoethyl)morpholine (0.07 mL, 0.51 mmol), 4-[2-(2-tert-butoxycarbonyl-2-azaspiro[3.3]heptan-6-yl)-5-[3-(methoxymethoxy)-1-naphthyl]-1-methyl-imidazol-4-yl]benzoic acid (200 mg, 0.34 mmol), DIPEA (0.18 mL, 1.03 mmol) and EtOAc (5 mL) at r.t. under a nitrogen atmosphere. The reaction was heated to 60° C. for 18 hours and then cooled to room temperature and solvent removed in vacuo. The residue was partitioned between sat. aq. NaHCO₃ (50 mL) and DCM (50 mL). The mixture as passed through a phase separator and the organic layer concentrated in vacuo and the residue purified by column chromatography using an eluent of 0-10% MeOH in DCM to give tert-butyl 6-[5-[3-(methoxymethoxy)-1-naphthyl]-1-methyl-4-[4-(2-morpholinoethylcarbamoyl)phenyl]imidazol-2-yl]-2-azaspiro[3.3]heptane-2-carboxylate (75 mg, 0.108 mmol, 31% yield) as an orange oil.

UPLC-MS (ES+, Method 1), 1.45 min, m/z 696.5 [M+H]⁺; 348.9 [M+2H]²⁺

### Step 4—4-[2-(2-azaspiro[3.3]heptan-6-yl)-5-(3-hydroxy-1-naphthyl)-1-methyl-imidazol-4-yl]-N-(2-morpholinoethyl)benzamide

tert-butyl 6-[5-[3-(methoxymethoxy)-1-naphthyl]-1-methyl-4-[4-(2-morpholinoethylcarbamoyl)phenyl]imidazol-2-yl]-2-azaspiro[3.3]heptane-2-carboxylate (75 mg, 0.11 mmol) was stirred in TFA (3.0 mL, 39.18 mmol) at room temperature for 2 hours. After this time the solvent was removed in vacuo and the mixture azeotroped with MeOH 3 times to give 4-[2-(2-azaspiro[3.3]heptan-6-yl)-5-(3-hydroxy-1-naphthyl)-1-methyl-imidazol-4-yl]-N-(2-morpholinoethyl)benzamide (59 mg, 0.107 mmol, 99% yield) as an orange oil which was used in the next step without further purification.

UPLC-MS (ES+, Method 1): 1.08 min, m/z 552.3 [M+H]⁺; 276.7 [M+2H]²⁺

### Step 5—4-[5-(3-hydroxy-1-naphthyl)-1-methyl-2-(2-prop-2-enoyl-2-azaspiro[3.3]heptan-6-yl)imidazol-4-yl]-N-(2-morpholinoethyl)benzamide—Example 29

Acryloyl chloride (0.01 mL, 0.11 mmol) was added to a stirred solution of 4-[2-(2-azaspiro[3.3]heptan-6-yl)-5-(3-hydroxy-1-naphthyl)-1-methyl-imidazol-4-yl]-N-(2-morpholinoethyl)benzamide (59. mg, 0.11 mmol), DIPEA (0.04 mL, 0.21 mmol), 1-Propanol (1 mL) and DCM (3 mL) with cooling in ice and the reaction stirred for 10 minutes. NH3 in MeOH (2 mL) was then added and the mixture stirred for 30 minutes. Water (30 mL) and DCM (30 mL) were added and the mixture was passed through a phase separator and the organic layer collected and solvent removed in vacuo. The residue was purified by column chromatography using an eluent of 0-10% MeOH in DCM to give 4-[5-(3-hydroxy-1-naphthyl)-1-methyl-2-(2-prop-2-enoyl-2-azaspiro[3.3]heptan-6-yl)imidazol-4-yl]-N-(2-morpholinoethyl)benzamide (3 mg, 0.005 mmol, 4% yield) as a white solid.

UPLC-MS (ES+, Method 2): 2.50 min, m/z 606.4 [M+H]⁺

¹H-NMR (400 MHz, CD3OD) δ/ppm: 7.78 (1H, d, J=8.4 Hz), 7.57 (2H, d, J=8.8 Hz), 7.46-7.39 (3H, m), 7.34-7.30 (2H, m), 7.24-7.20 (1H, m), 7.07 (1H, d, J=8.4 Hz), 6.43-6.34 (1H, m), 6.25 (1H, dt, J=16.8 Hz, 2.0 Hz), 5.78-5.74 (1H, m), 4.50 (1H, s), 4.36 (1H, s), 4.26 (1H, s), 4.13 (1H, s), 3.79-3.66 (5H, m), 3.49 (2H, t, J=6.4 Hz), 3.24 (3H, s), 2.90-2.75 (4H, m), 2.58-2.49 (6H, m), 2× exchangeable protons not seen.

### Example 38—1-[6-[5-(5,6-dimethyl-1H-indazol-4-yl)-1-methyl-4-(1-methylindazol-5-yl)imidazol-2-yl]-2-azaspiro[3.3]heptan-2-yl]prop-2-en-1-one

### Step 1—tert-butyl 6-[4-bromo-5-(5,6-dimethyl-1-tetrahydropyran-2-yl-indazol-4-yl)-1-methyl-imidazol-2-yl]-2-azaspiro[3.3]heptane-2-carboxylate

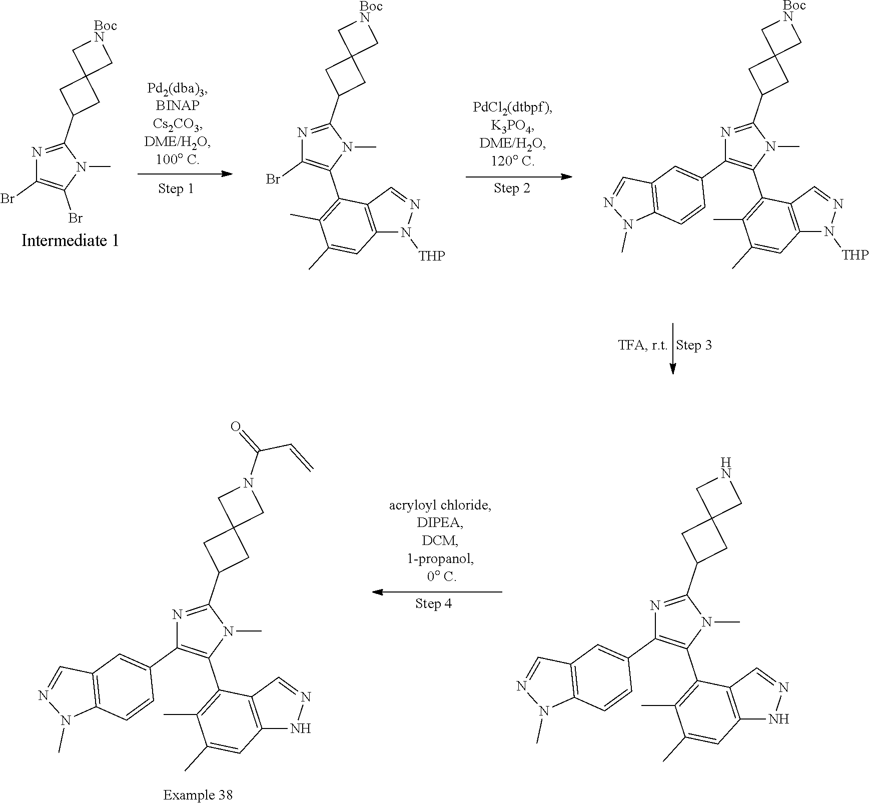

Intermediate 1 (250 mg, 0.57 mmol), Intermediate 16 (246 mg, 0.69 mmol), Cs₂CO₃ (749 mg, 2.3 mmol), Pd₂(dba)₃ (79 mg, 0.09 mmol), (+/−)-BINAP (54 mg, 0.09 mmol) in monoglyme (9.6 mL) and water (1 mL) are stirred at 100° C. for one hour in a sealed microwave vial under conventional heating conditions. The reaction was cooled down to room temperature and filtered through a celite pad. Solvent was evaporated and crude was purified using automated flash column chromatography using and eluent of 0-100% EtOAc in petroleum ether to give the tert-butyl 6-[4-bromo-5-(5,6-dimethyl-1-tetrahydropyran-2-yl-indazol-4-yl)-1-methyl-imidazol-2-yl]-2-azaspiro[3.3]heptane-2-carboxylate (140 mg, 0.24 mmol, 42% yield) as a yellow oil.

UPLC-MS (ES+, Method 3): 2.15 min, m/z 586.1 [M+H]⁺

### Step 2—tert-butyl 6-[5-(5,6-dimethyl-1-tetrahydropyran-2-yl-indazol-4-yl)-1-methyl-4-(1-methylindazol-5-yl)imidazol-2-yl]-2-azaspiro[3.3]heptane-2-carboxylate

1-Methyl-5-(4,4,5,5-tetramethyl-1,3,2-dioxaborolan-2-yl)-1H-indazole (99 mg, 0.38 mmol), potassium phosphate tribasic (203 mg, 0.96 mmol), tert-butyl 6-[4-bromo-5-(5,6-dimethyl-1-tetrahydropyran-2-yl-indazol-4-yl)-1-methyl-imidazol-2-yl]-2-azaspiro[3.3]heptane-2-carboxylate (140 mg, 0.24 mmol), Pd(dppf)Cl₂·CH₂Cl₂ (4 mg, 0.01 mmol) in 1,4-dioxane (5.33 mL) and water (0.66 mL) are stirred at 110° C. for 1 hour and then cooled down at room temperature and filtered through a celite pad. The filtrate was concentrated in vacuo and the residue purified by column chromatography using an eluent of 0-10% DCM in MeOH followed by reverse phase chromatography to give tert-butyl 6-[5-[5-(methoxymethoxy)-2-(trifluoromethyl)phenyl]-1-methyl-4-(1-methylindazol-5-yl)imidazol-2-yl]-2-azaspiro[3.3]heptane-2-carboxylate (30 mg, 0.049 mmol, 13% yield).

UPLC-MS (ES+, Method 3): 1.78 min, m/z 636.4 [M+H]⁺

### Step 3—4-[2-(2-azaspiro[3.3]heptan-6-yl)-3-methyl-5-(1-methylindazol-5-yl)imidazole-4-yl]-5,6-dimethyl-1H-indazole

tert-butyl 6-[5-(5,6-dimethyl-1-tetrahydropyran-2-yl-indazol-4-yl)-1-methyl-4-(1-methylindazol-5-yl)imidazol-2-yl]-2-azaspiro[3.3]heptane-2-carboxylate (50 mg, 0.08 mmol) and TFA (1.81 mL, 24 mmol) are stirred at 0° C. for 1 hour. Solvent was then removed in vacuo to give 4-[2-(2-azaspiro[3.3]heptan-6-yl)-3-methyl-5-(1-methylindazol-5-yl)imidazol-4-yl]-5,6-dimethyl-1H-indazole (35 mg, 0.07 mmol, 98% yield) as a yellow solid was used in the next step without further purification.

UPLC-MS (ES+, Method 3): 1.19 min, m/z 452.4 [M+H]⁺

### Step 4—1-[6-[5-(5,6-dimethyl-1H-indazol-4-yl)-1-methyl-4-(1-methylindazol-5-yl)121 midazole-2-yl]-2-azaspiro[3.3]heptan-2-yl]prop-2-en-1-one—Example 38

4-[2-(2-azaspiro[3.3]heptan-6-yl)-3-methyl-5-(1-methylindazol-5-yl)imidazol-4-yl]-5,6-dimethyl-1H-indazole (54 mg, 0.12 mmol), DIPEA (0.005 mL, 0.03 mmol) in DCM (3 mL) and 1-propanol (1 mL) are stirred at 0° C. A solution of acryloyl chloride (0.01 mL, 0.08 mmol) in DCM (0.95 mL) is added to the reaction mixture and stirred at 0° C. for 5 minutes. 7N NH₃ in MeOH (10 mL) was then added and reaction mixture was stirred at 0° C. for 30 minutes. The reaction mixture was then diluted with water (10 mL) and passed through a phase separator. Solvent was evaporated and crude was purified using flash column chromatography chromatography using and eluent of 0-10% MeOH in DCM on an amine based column Biotage (KD) column to give 1-[6-[5-(5,6-dimethyl-1H-indazol-4-yl)-1-methyl-4-(1-methylindazol-5-yl)imidazol-2-yl]-2-azaspiro[3.3]heptan-2-yl]prop-2-en-1-one (13 mg, 0.025 mmol, 21% yield) as a white powder.

UPLC-MS (ES+, Method 2): 2.64 min, m/z 506.4 [M+H]⁺

¹H-NMR (400 MHz, DMSO-d₆) δ/ppm: 12.98 (1H, s), 7.85 (1H, s), 7.75-7.70 (1H, m), 7.49 (1H, s), 7.41 (1H, s), 7.33-7.21 (2H, m), 6.32 (1H, dt, J=17.0 Hz, 9.7 Hz), 6.13-6.06 (1H, m), 5.67 (1H, ddd, J=10.0 Hz, 7.5 Hz, 2.3 Hz), 4.38 (1H, s), 4.24 (1H, s), 4.09 (1H, d, J=5.7 Hz), 3.95 (1H, s), 3.92 (3H, s), 3.70-3.60 (1H, m), 3.17 (1H, d, J=5.3 Hz), 3.09 (3H, s), 2.77-2.68 (1H, m), 2.33 (2H, q, J=1.9 Hz), 2.00 (3H, s).

### Example 39—1-[6-[5-[5-hydroxy-2-(trifluoromethyl)phenyl]-1-methyl-4-(1-methylindazol-5-yl)imidazol-2-yl]-2-azaspiro[3.3]heptan-2-yl]prop-2-en-1-one

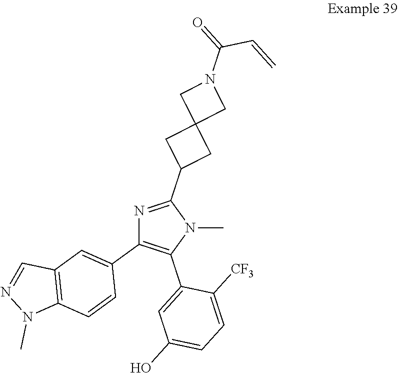 [SMILES: C=CC(=O)N1CC2(CC(c3nc(-c4ccc5c(cnn5C)c4)c(-c4cc(O)ccc4C(F)(F)F)n3C)C2)C1]

This Example was made according to the reaction Scheme for Example 38, substituting Intermediate 17 in place of Intermediate 16.

### Step 1—tert-butyl 6-[4-bromo-5-[5-(methoxymethoxy)-2-(trifluoromethyl)phenyl]-1-methyl-imidazol-2-yl]-2-azaspiro[3.3]heptane-2-carboxylate

tert-butyl 6-(4,5-dibromo-1-methyl-imidazol-2-yl)-2-azaspiro[3.3]heptane-2-carboxylate (250 mg, 0.57 mmol), 2-[5-(methoxymethoxy)-2-(trifluoromethyl)phenyl]-4,4,5,5-tetramethyl-1,3,2-dioxaborolane (229 mg, 0.69 mmol), Cs₂CO₃ (749 mg, 2.3 mmol), Pd₂(dba)₃ (79 mg, 0.09 mmol), (+/−)-BINAP (54 mg, 0.09 mmol) in monoglyme (13 mL) and water (1.3 mL) are stirred at 100° C. for one hour in a sealed microwave vial under conventional heating conditions. The reaction mixture was cooled down to r.t. and filtered through a celite pad. The filtrate was concentrated in vacuo and the residue purified by column chromatography using an eluent of 0-100% EtOAc in petroleum ether to give tert-butyl 6-[4-bromo-5-[5-(methoxymethoxy)-2-(trifluoromethyl)phenyl]-1-methyl-imidazol-2-yl]-2-azaspiro[3.3]heptane-2-carboxylate (210 mg, 0.375 mmol, 65% yield) as a yellow oil.

UPLC-MS (ES⁺, Method 3): 2.11 min, m/z 572.2 [M+H]⁺

### Step 2—tert-butyl 6-[5-[5-(methoxymethoxy)-2-(trifluoromethyl)phenyl]-1-methyl-4-(1-methylindazol-5-yl)122midazole-2-yl]-2-azaspiro[3.3]heptane-2-carboxylate

1-Methyl-5-(4,4,5,5-tetramethyl-1,3,2-dioxaborolan-2-yl)-1H-indazole (221 mg, 0.86 mmol), K₃PO₄ (485 mg, 2.28 mmol), tert-butyl 6-[4-bromo-5-[5-(methoxymethoxy)-2-(trifluoromethyl)phenyl]-1-methyl-imidazol-2-yl]-2-azaspiro[3.3]heptane-2-carboxylate (320 mg, 0.57 mmol), PdCl₂(dtbpf) (9 mg, 0.01 mmol) in 1,4-dioxane (12.71 mL) and water (1.56 mL) are stirred at 110° C. for 1 hour. The reaction was then cooled to r.t. and the mixture filtered through a celite pad. The filtrate was concentrated in vacuo and the residue purified by column chromatography using an eluent of 0-10% MeOH in DCM. Product containing fractions were collected and concentrated in vacuo and the residue re-purified by prep-HPLC to give tert-butyl 6-[5-[5-(methoxymethoxy)-2-(trifluoromethyl)phenyl]-1-methyl-4-(1-methylindazol-5-yl)imidazol-2-yl]-2-azaspiro[3.3]heptane-2-carboxylate (30 mg, 0.049 mmol, 13% yield).

UPLC-MS (ES⁺, Method 3): 1.71/1.75 min, m/z 612.3 [M+H]⁺

### Step 3—3-[2-(2-azaspiro[3.3]heptan-6-yl)-3-methyl-5-(1-methylindazol-5-yl)123midazole-4-yl]-4-(trifluoromethyl)phenol

tert-butyl 6-[5-[5-(methoxymethoxy)-2-(trifluoromethyl)phenyl]-1-methyl-4-(1-methylindazol-5-yl)imidazol-2-yl]-2-azaspiro[3.3]heptane-2-carboxylate (40 mg, 0.07 mmol) and TFA (1 mL, 13.06 mmol) are stirred at for 2 hours. Solvent was then evaporated to give 3-[2-(2-azaspiro[3.3]heptan-6-yl)-3-methyl-5-(1-methylindazol-5-yl)imidazol-4-yl]-4-(trifluoromethyl)phenol (30 mg, 0.06 mmol, 98% yield) as a yellow oil was used directly in the next step without purification.

UPLC-MS (ES⁺, Method 1): 1.10 min, m/z 468.3 [M+H]⁺′

### Step 4—1-[6-[5-[5-hydroxy-2-(trifluoromethyl)phenyl]-1-methyl-4-(1-methylindazol-5-yl)imidazol-2-yl]-2-azaspiro[3.3]heptan-2-yl]prop-2-en-1-one—Example 39

3-[2-(2-azaspiro[3.3]heptan-6-yl)-3-methyl-5-(1-methylindazol-5-yl)imidazol-4-yl]-4-(trifluoromethyl)phenol (30 mg, 0.06 mmol), DIPEA (0.003 mL, 0.02 mmol) in DCM (1.2 mL) and 1-propanol (0.4 mL) are stirred at 0° C. A solution of acryloyl chloride (0.01 mL, 0.06 mmol) in DCM (0.39 mL) is added to the reaction mixture and stirred at 0° C. for 5 minutes. 7N NH₃ in MeOH (10 mL) was then added and reaction mixture was stirred at 0° C. for 30 minutes. The reaction mixture was then diluted with water (10 mL) and passed through a phase separator. Solvent was evaporated and the residue was purified using flash column chromatography chromatography using and eluent of 0-10% MeOH in DCM on an amine based Biotage (KD) column. Product containing fractions were then combined and concentrated in vacuo and the residue re-purified by prep-HPLC to give 1-[6-[5-[5-hydroxy-2-(trifluoromethyl)phenyl]-1-methyl-4-(1-methylindazol-5-yl)imidazol-2-yl]-2-azaspiro[3.3]heptan-2-yl]prop-2-en-1-one (6 mg, 0.011 mmol, 18% yield) as a white powder.

UPLC-MS (ES⁺, Method 2): 2.81 min, m/z 522.3 [M+H]⁺

¹H NMR (400 MHz, DMSO-d₆) δ/ppm: 7.91 (1H, s,), 7.72 (2H, s), 7.42 (1H, d, J=8.9 Hz), 7.35 (1H, d, J=8.7 Hz), 7.06 (1H, s), 6.80 (1H, s), 6.35-6.20 (1H, m), 6.09 (1H, d, J=16.8 Hz), 5.66 (1H, t, J=8.9 Hz), 4.37 (1H, s), 4.21 (1H, s), 4.09 (3H, q, J=5.2 Hz), 3.96 (3H, s), 3.92 (1H, s), 3.17 (2H, s), 3.13 (3H, s).

### Example 40—give 4-[3-methyl-5-(1-methylindazol-5-yl)-2-(2-prop-2-enoyl-2-azaspiro[3.3]heptan-6-yl)imidazol-4-yl]-1H-indazole-6-carbonitrile

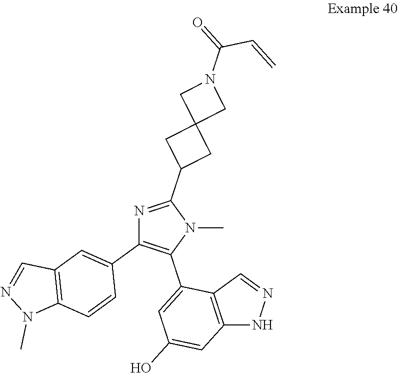 [SMILES: C=CC(=O)N1CC2(CC(c3nc(-c4ccc5c(cnn5C)c4)c(-c4cc(O)cc5[nH]ncc45)n3C)C2)C1]

This Example was made according to the reaction Scheme for Example 38, substituting Intermediate 18 in place of Intermediate 16.

### Step 1—tert-butyl 6-[4-bromo-5-(6-cyano-1-tetrahydropyran-2-yl-indazol-4-yl)-1-methyl-imidazol-2-yl]-2-azaspiro[3.3]heptane-2-carboxylate

(+/−)-BINAP (54 mg, 0.09 mmol), Intermediate 18 (243 mg, 0.69 mmol), Cs₂CO₃ (1.12 g, 3.45 mmol), Intermediate 1 (250 mg, 0.57 mmol), Pd₂(dba)₃ (79 mg, 0.09 mmol), monoglyme (4 mL) and water (1 mL) were combined in a sealable vial and stirred. The mixture was bubbled through with nitrogen for 1 minute and then the vial sealed and heated to 100° C. for 2 hours. It was then cooled to room temperature, combined and solvent removed in vacuo. The residue was taken up in DCM (5 mL) and filtered through celite and the filter cake washed with DCM (5 mL). The filtrate was concentrated in vacuo and the residue purified by column chromatography using an eluent of 0-100% EtOAc in petroleum ether to give tert-butyl 6-[4-bromo-5-(6-cyano-1-tetrahydropyran-2-yl-indazol-4-yl)-1-methyl-imidazol-2-yl]-2-azaspiro[3.3]heptane-2-carboxylate (250 mg, 0.430 mmol, 75% yield) as a yellow solid.

UPLC-MS (ES+, Method 1): 2.09 min, m/z 581.2, 583.2 [M+H]⁺

### Step 2—tert-butyl 6-[5-(6-cyano-1-tetrahydropyran-2-yl-indazol-4-yl)-1-methyl-4-(1-methylindazol-5-yl)imidazol-2-yl]-2-azaspiro[3.3]heptane-2-carboxylate

1-Methyl-5-(4,4,5,5-tetramethyl-1,3,2-dioxaborolan-2-yl)-1H-indazole (133.17 mg, 0.52 mmol), tert-butyl 6-[4-bromo-5-(6-cyano-1-tetrahydropyran-2-yl-indazol-4-yl)-1-methyl-imidazol-2-yl]-2-azaspiro[3.3]heptane-2-carboxylate (250 mg, 0.43 mmol), PdCl₂(dtbpf) (28 mg, 0.04 mmol), K₃PO₄ (456 mg, 2.15 mmol), 1,4-dioxane (4.5 mL) and water (0.5 mL) were combined and stirred in a sealable vial. Nitrogen was bubbled through the reaction mixture for 1 minute and then the vial sealed and stirred at 110° C. for for 2 hours. The reaction was then cooled to room temperature and solvent removed in vacuo. The residue was taken up in DCM (10 mL) and filtered through celite and the filter cake washed with DCM (10 mL). The filtrated was concentrated in vacuo and the residue purified by column chromatography using an eluent of 0-100% EtOAc in petroleum ether to give tert-butyl 6-[5-(6-cyano-1-tetrahydropyran-2-yl-indazol-4-yl)-1-methyl-4-(1-methylindazol-5-yl)125midazole-2-yl]-2-azaspiro[3.3]heptane-2-carboxylate (173 mg, 0.273 mmol, 63% yield) a yellow oil.

UPLC-MS (ES+, Method 3): 1.73 min, m/z 633.4 [M+H]⁺

### Step 3—4-[2-(2-azaspiro[3.3]heptan-6-yl)-3-methyl-5-(1-methylindazol-5-yl)imidazol-4-yl]-1H-indazole-6-carbonitrile

tert-butyl 6-[5-(6-cyano-1-tetrahydropyran-2-yl-indazol-4-yl)-1-methyl-4-(1-methylindazol-5-yl)imidazol-2-yl]-2-azaspiro[3.3]heptane-2-carboxylate (173 mg, 0.27 mmol) was stirred in TFA (3 mL, 39.18 mmol) at room temperature for 1 hour. After this time the solvent was removed in vacuo and the mixture azeotroped with MeOH 3 times to give 4-[2-(2-azaspiro[3.3]heptan-6-yl)-3-methyl-5-(1-methylindazol-5-yl)imidazol-4-yl]-1H-indazole-6-carbonitrile (122 mg, 0.272 mmol, 100% yield) as an orange oil which was used in the next step without further purification.

UPLC-MS (ES+, Method 3): 1.10 min, m/z 449.2 [M+H]⁺

### Step 4—4-[3-methyl-5-(1-methylindazol-5-yl)-2-(2-prop-2-enoyl-2-azaspiro[3.3]heptan-6-yl)imidazol-4-yl]-1H-indazole-6-carbonitrile—Example 40

Acryloyl chloride (0.02 mL, 0.27 mmol) was added to a stirred solution of 4-[2-(2-azaspiro[3.3]heptan-6-yl)-3-methyl-5-(1-methylindazol-5-yl)imidazol-4-yl]-1H-indazole-6-carbonitrile (122 mg, 0.27 mmol), DIPEA (0.14 mL, 0.82 mmol), 1-propanol (1 mL) and DCM (3 mL) with cooling in ice and the reaction stirred for 10 minutes. 7N NH₃ in MeOH (2 mL) was then added and the mixture stirred for 30 minutes. Water (30 mL) and DCM (30 mL) were added and the mixture was passed through a phase separator and the organic layer collected and solvent removed in vacuo. The residue was purified by column chromatography using an eluent of 0-10% MeOH in DCM to give 4-[3-methyl-5-(1-methylindazol-5-yl)-2-(2-prop-2-enoyl-2-azaspiro[3.3]heptan-6-yl)imidazol-4-yl]-1H-indazole-6-carbonitrile (12 mg, 0.024 mmol, 9% yield) as a white solid.

UPLC-MS (ES+, Method 2): 2.42 min, m/z 503.3 [M+H]⁺

¹H-NMR (400 MHz, DMSO-d₆) δ/ppm: 13.75 (1H, s), 8.26 (1H, s), 7.89 (1H, s), 7.69-7.65 (2H, m), 7.55-7.53 (1H, m), 7.35 (1H, d, J=8.4 Hz), 7.21 (1H, dd, J=8.4 Hz, 1.2 Hz), 6.38-6.27 (1H, m), 6.14-6.05 (1H, m), 5.70-5.63 (1H, m), 4.39 (1H, s), 4.23 (1H, s), 4.10 (1H, s), 3.97-3.92 (4H, m), 3.70-3.59 (1H, m), 3.32 (3H, s), 2.72-2.58 (4H, m).

### Example 41—1-[6-[5-(6-chloro-1H-indazol-4-yl)-1-methyl-4-(1-methylindazol-5-yl)imidazol-2-yl]-2-azaspiro[3.3]heptan-2-yl]prop-2-en-1-one

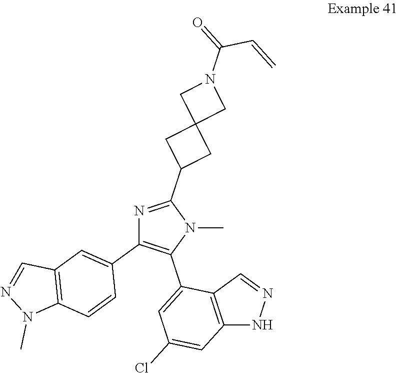 [SMILES: C=CC(=O)N1CC2(CC(c3nc(-c4ccc5c(cnn5C)c4)c(-c4cc(Cl)cc5[nH]ncc45)n3C)C2)C1]

### Example 41

This Example was made according to the reaction Scheme for Example 38, substituting Intermediate 19 in place of Intermediate 16.

### Step 1—tert-butyl 6-[4-bromo-5-(6-chloro-1-tetrahydropyran-2-yl-indazol-4-yl)-1-methyl-imidazol-2-yl]-2-azaspiro[3.3]heptane-2-carboxylate

(+/−)-BINAP (54 mg, 0.09 mmol), Intermediate 19 (250 mg, 0.69 mmol), Cs₂CO₃ (1123 mg, 3.45 mmol), Intermediate 1 (250 mg, 0.57 mmol), Pd₂(dba)₃ (79 mg, 0.09 mmol), monoglyme (4 mL) and water (1 mL) were combined in a sealable vial and stirred. The mixture was bubbled through with nitrogen for 1 minute and then the vial sealed and heated to 100° C. for 2 hours. It was then cooled to room temperature, combined and solvent removed in vacuo. The residue was taken up in DCM (5 mL) and filtered through celite and the filter cake washed with DCM (5 mL). The filtrate was concentrated in vacuo and the residue purified by column chromatography using an eluent of 0-100% EtOAc in petroleum ether to give tert-butyl 6-[4-bromo-5-(6-chloro-1-tetrahydropyran-2-yl-indazol-4-yl)-1-methyl-imidazol-2-yl]-2-azaspiro[3.3]heptane-2-carboxylate (254 mg, 0.430 mmol, 75% yield) as a yellow solid.

UPLC-MS (ES+, Method 1): 2.17 min, m/z 590.3, 592.3, 594.2 [M+H]⁺

### Step 2—tert-butyl 6-[5-(6-chloro-1-tetrahydropyran-2-yl-indazol-4-yl)-1-methyl-4-(1-methylindazol-5-yl)imidazol-2-yl]-2-azaspiro[3.3]heptane-2-carboxylate

1-Methyl-5-(4,4,5,5-tetramethyl-1,3,2-dioxaborolan-2-yl)-1H-indazole (133 mg, 0.52 mmol), tert-butyl 6-[4-bromo-5-(6-chloro-1-tetrahydropyran-2-yl-indazol-4-yl)-1-methyl-imidazol-2-yl]-2-azaspiro[3.3]heptane-2-carboxylate (254 mg, 0.43 mmol), PdCl₂(dtbpf), K₃PO₄ (457 mg, 2.15 mmol), 1,4-dioxane (4.5 mL) and water (0.5 mL) were combined and stirred in a sealable vial. Nitrogen was bubbled through the reaction mixture for 1 minute and then the vial sealed and stirred at 110° C. for 2 hours. The reaction was then cooled to room temperature and solvent removed in vacuo. The residue was taken up in DCM (10 mL) and filtered through celite and the filter cake washed with DCM (10 mL). The filtrated was concentrated in vacuo and the residue purified by column chromatography using an eluent of 0-100% EtOAc in petroleum ether to give tert-butyl 6-[5-(6-chloro-1-tetrahydropyran-2-yl-indazol-4-yl)-1-methyl-4-(1-methylindazol-5-yl)imidazol-2-yl]-2-azaspiro[3.3]heptane-2-carboxylate (55 mg, 0.086 mmol, 20% yield) a yellow oil.

UPLC-MS (ES+, Method 3): 1.81 min, m/z 642.3, 644.3 [M+H]⁺

### Step 3—4-[2-(2-azaspiro[3.3]heptan-6-yl)-3-methyl-5-(1-methylindazol-5-yl)imidazol-4-yl]-6-chloro-1H-indazole

tert-butyl 6-[5-(6-chloro-1-tetrahydropyran-2-yl-indazol-4-yl)-1-methyl-4-(1-methylindazol-5-yl)imidazol-2-yl]-2-azaspiro[3.3]heptane-2-carboxylate (55 mg, 0.09 mmol) was stirred in TFA (3. mL, 39.18 mmol) at room temperature for 1 hour. After this time the solvent was removed in vacuo and the mixture azeotroped with MeOH 3 times to give 4-[2-(2-azaspiro[3.3]heptan-6-yl)-3-methyl-5-(1-methylindazol-5-yl)imidazol-4-yl]-6-chloro-1H-indazole (39 mg, 0.085 mmol, 99% yield) as an orange oil which was used in the next step without further purification.

UPLC-MS (ES+, Method 3): 1.24 min, m/z 458.1, 460.1 [M+H]⁺

### Step 4—1-[6-[5-(6-chloro-1H-indazol-4-yl)-1-methyl-4-(1-methylindazol-5-yl)imidazol-2-yl]-2-azaspiro[3.3]heptan-2-yl]prop-2-en-1-one—Example 41

Acryloyl chloride (0.01 mL, 0.09 mmol) was added to a stirred solution of 4-[2-(2-azaspiro[3.3]heptan-6-yl)-3-methyl-5-(1-methylindazol-5-yl)imidazol-4-yl]-6-chloro-1H-indazole (39. mg, 0.09 mmol), DIPEA (0.04 mL, 0.26 mmol), 1-propanol (1 mL) and DCM (3 mL) with cooling in ice and the reaction stirred for 10 minutes. 7N NH₃ in MeOH (2 mL) was then added and the mixture stirred for 30 minutes. Water (30 mL) and DCM (30 mL) were added and the mixture was passed through a phase separator and the organic layer collected and solvent removed in vacuo. The residue was purified by column chromatography using an eluent of 0-10% MeOH in DCM to give 1-[6-[5-(6-chloro-1H-indazol-4-yl)-1-methyl-4-(1-methylindazol-5-yl)imidazol-2-yl]-2-azaspiro[3.3]heptan-2-yl]prop-2-en-1-one (3 mg, 0.006 mmol, 7% yield) as a white solid.

UPLC-MS (ES+, Method 2): 2.65 min, m/z 512.3, 514.2 [M+H]⁺

¹H-NMR (400 MHz, DMSO-d₆) δ/ppm: 13.32 (1H, s), 7.89 (1H, s), 7.73-7.70 (2H, m), 7.58-7.55 (1H, m), 7.38-7.35 (1H, m), 7.25-7.18 (2H, m), 6.37-6.26 (1H, m), 6.13-6.06 (1H, m), 5.70-5.62 (1H, m), 4.39 (1H, s), 4.23 (1H, s), 4.09 (1H, s), 3.96-3.91 (4H, m), 3.69-3.58 (1H, m), 3.31 (3H, s), 2.75-2.58 (4H, m).

### Compound Depletion Assay Method

Lyophilized human K-RAS (12 Cys) protein (His tag) and human K-RAS (12 Asp) protein (His tag) were purchased from Stratech and reconstituted at 30 μM in water. The protein concentration of K-RAS G12C and K-RAS G12D was adjusted to 20 μM in K-RAS Assay Buffer (20 mM HEPES pH 7.5, 150 mM NaCl, 1 mM MgCl₂). A 49 μl aliquot of protein solution was transferred to 1.5 ml eppendorfs and incubated at room temperature for 10 minutes. A 1 μl aliquot of 400 μM compound solution in DMSO was added to the protein solutions in the 1.5 ml eppendorfs to initiate the reaction, with a final assay compound concentration of 8 μM. Reactions were agitated at 25° C., 700 rpm. At each time point (0, 30, 180, 360 minutes), 10 μl of the reaction mixture was removed, added to 30 μl quench solution (0.1% formic acid, 0.1 μM labetalol and 0.1 μM propranolol in acetonitrile) to stop the reaction and stored at 4° C. Once all reactions were quenched, the samples were centrifuged at 16000 g for 10 minutes to pellet the protein and salts. A 10 μl aliquot of supernatant containing compound was diluted in 50:50 acetonitrile:H₂O in a 96 well plate. The plates were heat sealed and the samples analysed by LC-MS/MS to determine the concentration of compound remaining in the samples. To determine the reaction progress, the compound signal was normalized to the internal standards signal (labetalol and propranolol). The decrease in free compound concentration is shown as a percentage over time, with the percent engagement at time zero fixed at one hundred percent. Compound is assumed to be completely depleted when no longer detectable with a double blank sample as reference.

### Compound Depletion Assay Data

| | 30 min | 3 hr | 6 hr | |
|---|---|---|---|---|
| Example | Category | Category | Category | Protein |
| 1 | B | B | B | G12C |
| 2 | A | B | B | G12C |
| 3 | — | — | — | G12C |
| 4 | A | B | B | G12C |
| 5 | B | B | B | G12C |
| 6 | — | — | — | G12C |
| 7 | A | B | B | G12C |
| 8 | — | — | — | G12C |
| 9 | A | B | B | G12C |
| 10 | — | — | — | G12C |
| 11 | A | B | B | G12C |
| 12 | A | A | A | G12C |
| 13 | A | A | B | G12C |
| 14 | B | B | B | G12C |
| 15 | — | — | — | G12C |
| 16 | A | B | B | G12C |
| 17 | A | A | B | G12C |
| 18 | B | B | B | G12C |
| 19 | — | — | — | G12C |
| 20 | — | — | — | G12C |
| 21 | — | — | — | G12C |
| 22 | B | B | B | G12C |
| 23 | — | — | — | G12C |
| 24 | B | B | B | G12C |
| 25 | A | A | A | G12C |
| 26 | B | B | B | G12C |
| 27 | B | B | B | G12C |
| 28 | B | B | B | G12C |
| 29 | — | — | — | G12C |
| 30 | B | B | B | G12C |
| 31 | — | — | — | G12C |
| 32 | — | — | — | G12C |
| 33 | — | — | — | G12C |
| 34 | A | A | A | G12C |
| 35 | B | B | B | G12C |
| 36 | A | A | A | G12C |
| 37 | — | — | — | G12C |
| 38 | B | B | B | G12C |
| 39 | — | — | — | G12C |
| 40 | A | B | C | G12C |
| 41 | C | C | C | G12C |

Data categories:
A—≥70% compound remaining at timepoint
B—30%-70% compound remaining at timepoint
C—≤30% compound remaining at timepoint

### HTRF Nucleotide Exchange Assay Method

The capacity of compounds to bind KRAS G12C and KRAS wildtype was quantified using a HTRF nucleotide exchange assay. Recombinant human KRAS protein (5 nM; aa1-188 KRAS WT or KRAS containing the G12C amino acid substitution, an N-terminal 6×His-tag and leader sequence), and 2 nM Europium-labeled anti-6×His antibody were mixed in assay buffer (10 mM HEPES pH7.3, 75 mM NaCl, 5 mM MgCl₂, 0.05% BSA, 0.0025% NP-40 and 100 mM KF) with various concentrations of compound in a 384-well plate and a volume of 5 μL. After a 180 minute incubation at room temperature, 5 μl of 200 nM EDA-GTP-DY647P1 diluted in assay buffer was added to the plate. Following 30-minute incubation at room temperature, time-resolved fluorescence was measured on a PerkinElmer Envision plate reader. DMSO (0.3%) and unlabeled GDP (1 μM) or equivalent tool compound were used to generate the Max and Min assay signals, respectively. Data was analysed using a four-parameter logistic model to calculate IC₅₀ values, with at least two independent replicates were performed for each compound.

| | KRAS G12C |
|---|---|
| | HTRF NEA |
| Example | IC₅₀ |
| 1 | C |
| 2 | B |
| 3 | A |
| 4 | B |
| 5 | C |
| 6 | B |
| 7 | B |
| 8 | B |
| 9 | B |
| 10 | A |
| 11 | B |
| 12 | A |
| 13 | A |
| 14 | C |
| 15 | A |
| 16 | B |
| 17 | B |
| 18 | C |
| 19 | B |
| 20 | B |
| 21 | B |
| 22 | B |
| 23 | B |
| 24 | C |
| 25 | A |
| 26 | C |
| 27 | C |
| 28 | C |
| 29 | C |
| 30 | C |
| 31 | C |
| 32 | C |
| 33 | C |
| 34 | A |
| 35 | C |
| 36 | B |
| 37 | C |
| 38 | C |
| 39 | C |
| 40 | B |
| 41 | C |

Data categories:
A—≥10,000 nM
B—1001-9999 nM
C—≤1000 nM

### INCORPORATION BY REFERENCE

All references, articles, publications, patents, patent publications, and patent applications cited herein within the above text are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

What is claimed is:

1. A compound of Formula (I-1) or Formula (I-2): 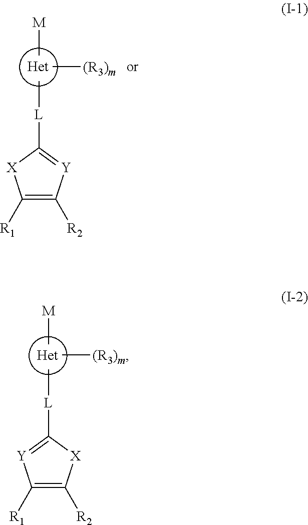
or a pharmaceutically acceptable salt, stereoisomer, or atropisomer thereof,
wherein:
X is N—R₄, O, or S;
Y is N or CH; 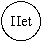
is a nitrogen-containing heterocyclyl;
L is a bond, alkylene, alkenylene, alkynylene, —C(O)—, or —S(O)₂—;
R₁ and R₂ are each independently aryl or heteroaryl;
R₃ is hydrogen, halogen, alkyl, hydroxy, alkoxy, —CN, —C(O)ORS, or —S(O)₂NR₅R₆; or
two R₃ groups attached to the same carbon atom form an oxo, cycloalkyl, or heterocyclyl; or two R₃ groups taken together with the carbon atoms to which they are attached form a cycloalkyl or heterocyclyl;
R₄ is alkyl, cycloalkyl, alkylenecycloalkyl, heterocyclyl, or alkyleneheterocyclyl;
R₅ and R₆ are each independently alkyl, cycloalkyl, heterocyclyl, aryl, heteroaryl, alkylenecycloalkyl, alkyleneheterocyclyl, alkylenearyl, or alkyleneheteroaryl;
M is 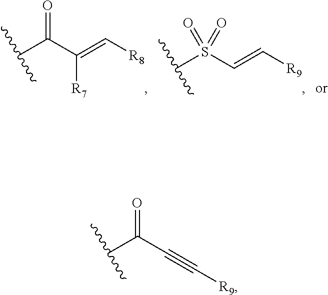
wherein
R₇ is hydrogen, halogen, C₁₋₃alkyl, —C(O)—C₁₋₃alkyl, or —CN;
R₈ is H, alkyl, —CF₃, —CHF₂, —CH₂F, —CH₂—Oalkyl, or —CH₂N(alkyl)₂; and
R₉ is hydrogen or alkyl; and
m is 0, 1, or 2.

2. The compound of claim 1, wherein 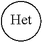
is a nitrogen-containing heterocyclyl comprising 1-3 heteroatoms.

3. The compound of claim 1 or 2, wherein 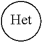
is a spirocyclic heterocyclyl comprising 1 or 2 heteroatoms atoms.

4. The compound of claim 1 or 2, wherein 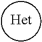
is: 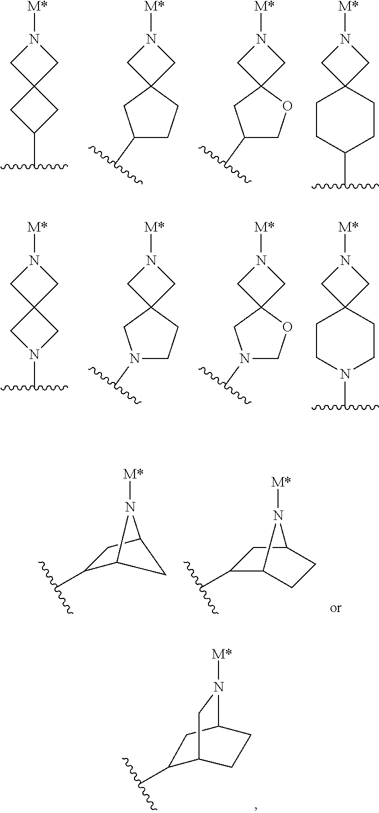
wherein M* represents the point of attachment to M and [symbol] represents the point of attachment to L.

5. The compound of any one of claims 1-3, wherein 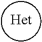
is: 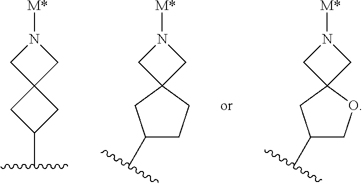
wherein M* represents the point of attachment to M and [symbol] represents the point of attachment to L.

6. The compound of any one of claims 1-5, wherein R₁ is a phenol, napthol, napthyl, or heteroaryl comprising 1, 2, or 3 heteroatoms selected from the group consisting of N, O, and S.

7. The compound of any one of claims 1-6, wherein R₁ is: 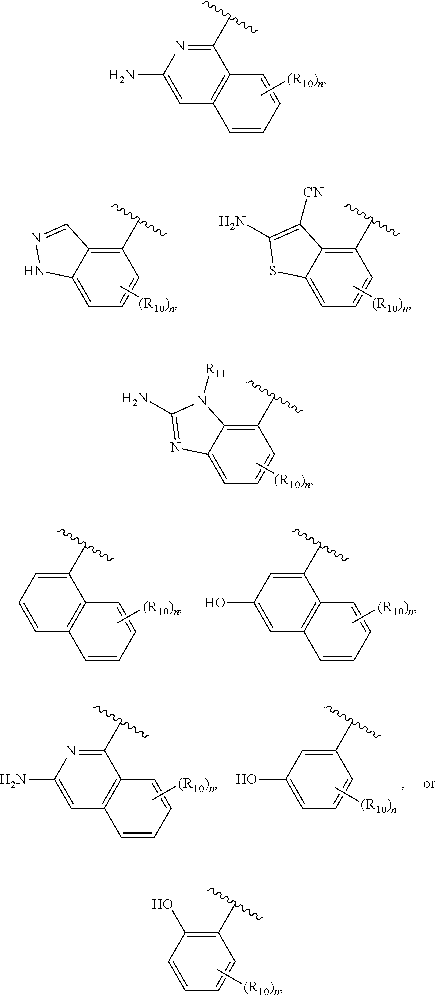
wherein:
R₁₀ is each independently halogen, alkyl, alkenyl, alkynyl, alkoxy, —CN, or cycloalkyl;
R₁₁ is H, alkyl, or cycloalkyl; and
n is 0, 1, 2, or 3.

8. The compound of any one of claims 1-7, wherein R₁ is: 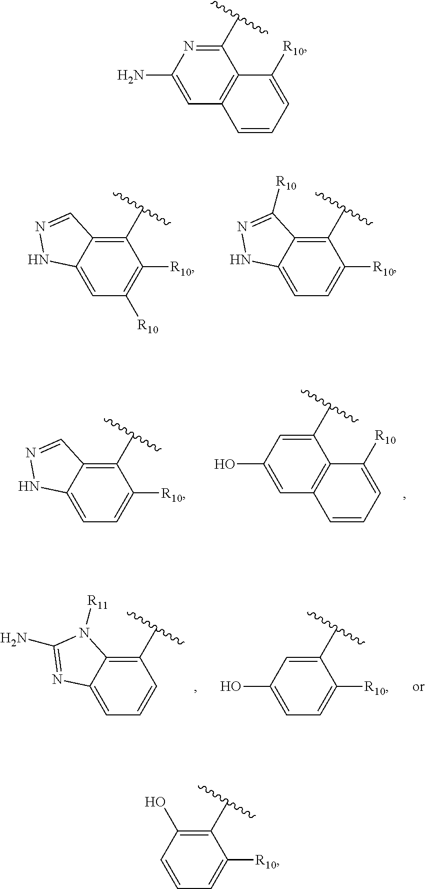
wherein:
R₁₀ is each independently halogen, alkyl, alkenyl, alkynyl, or alkoxy;
R₁₁ is H or alkyl; and
n is 0, 1, 2, or 3.

9. The compound of claim 7 or 8, wherein each R₁₀ is independently halogen, alkyl, or alkynyl.

10. The compound of any one of claims 7-9, wherein R₁₁ is methyl.

11. The compound of any one of claims 6-10, wherein n is 0, 1, or 2.

12. The compound of any one of claims 1-11, wherein R₁ is:

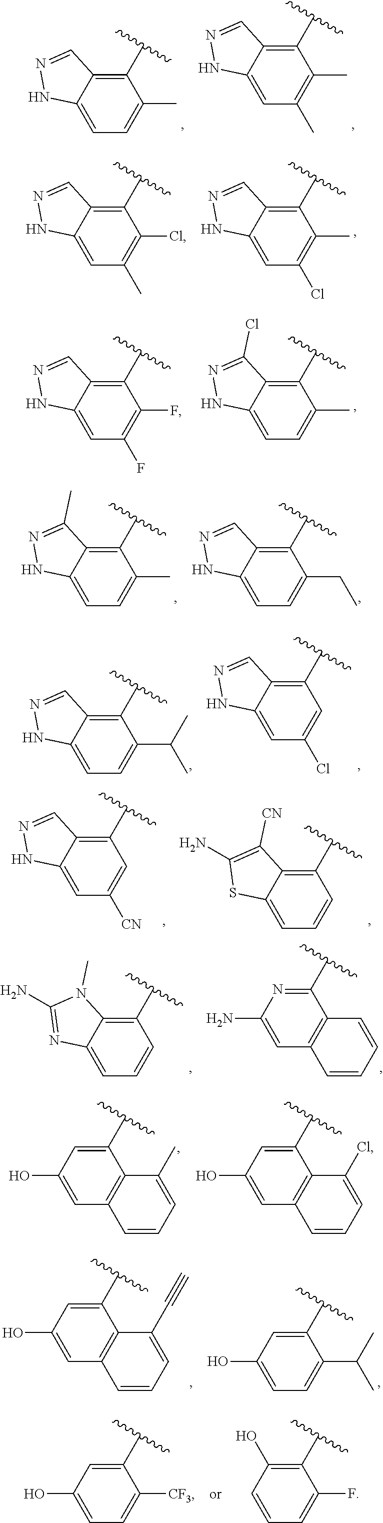

13. The compound of any one of claims 1-12, wherein R₂ is a substituted or unsubstituted phenyl or nitrogen-containing heteroaryl.

14. The compound of claim 13, wherein the nitrogen-containing heteroaryl is a fused bicyclic heteroaryl ring.

15. The compound of any one of claims 1-14, wherein R₂ is: 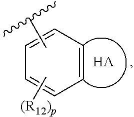
wherein: 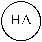
is a 5- or 6-membered nitrogen-containing heteroaryl ring;
R₁₂ is each independently halogen or alkyl; and
p is 0, 1, or 2.

16. The compound of any one of claims 1-15, wherein R₂ is: 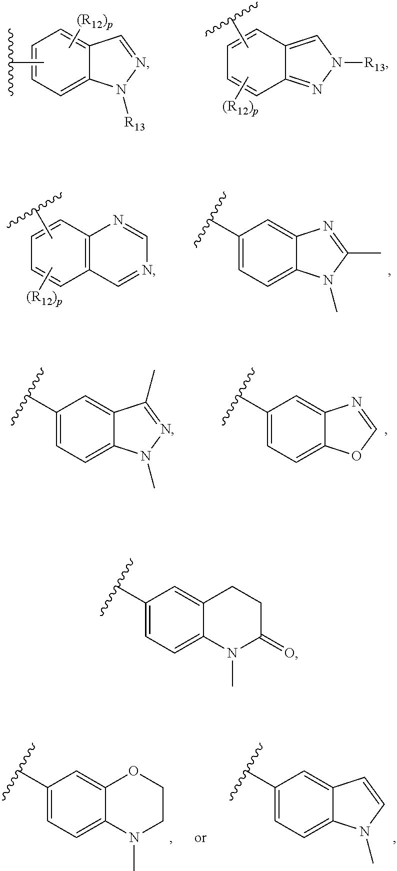
wherein:
R₁₂ is each independently halogen, alkyl, or cycloalkyl;
R₁₃ is alkyl or cycloalkyl; and
p is 0, 1, or 2.

17. The compound of claim 15 or 16, wherein R₁₂ is each independently F, Cl, or C₁₋₅alkyl.

18. The compound of any one of claims 15-17, wherein R₁₂ is each independently F, Cl, —CH₃, —CH₂CH₃, —CH(CH₃)₂, —CH₂CH(CH₃)₂, —C(CH₃)₃, or —CF₃.

19. The compound of any one of claims 16-18, wherein R₁₃ is C₁₋₅alkyl.

20. The compound of any one of claims 16-19, wherein R₁₃ is —CH₃, —CH₂CH₃, or —CH(CH₃)₂.

21. The compound of any one of claims 16-20, wherein p is 0 or 1.

22. The compound of any one of claims 1-14, wherein R₂ is: 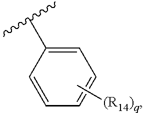
wherein:
R₁₄ is halogen, alkyl, alkoxy, —O—(C₂₋₄alkylene)-O-alkyl, —O—(C₁₋₃alkylene)-C(O)NR₁₅R₁₆, —CO₂alkyl, —C(O)NR₁₅R₁₆, —C(O)NH—(C₂₋₄alkylene)-NR₁₅R₁₆, —N(H)C(O)alkyl, —NH—(C₂₋₄alkylene)-NR₁₅R₁₆, cycloalkyl, heterocyclyl, —CH₂-heterocyclyl, aryl, or heteroaryl, or two R₁₄ groups taken together with the carbon atoms to which they are attached form a heterocyclyl or heteroaryl;
R₁₅ and R₁₆ are each independently H, alkyl, or fluoroalkyl, or an R₁₅ and R₁₆ taken together with the nitrogen atom to which they are attached form a heterocyclyl; and
q is 0, 1 or 2.

23. The compound of claim 22, wherein R₁₄ is heterocyclyl or —CH₂-heterocyclyl.

24. The compound of claim 22 or 23, wherein the heterocyclyl is morpholino or N-methylpiperazinyl, and q is 1.

25. The compound of any one of claims 1-14, wherein R₂ is:

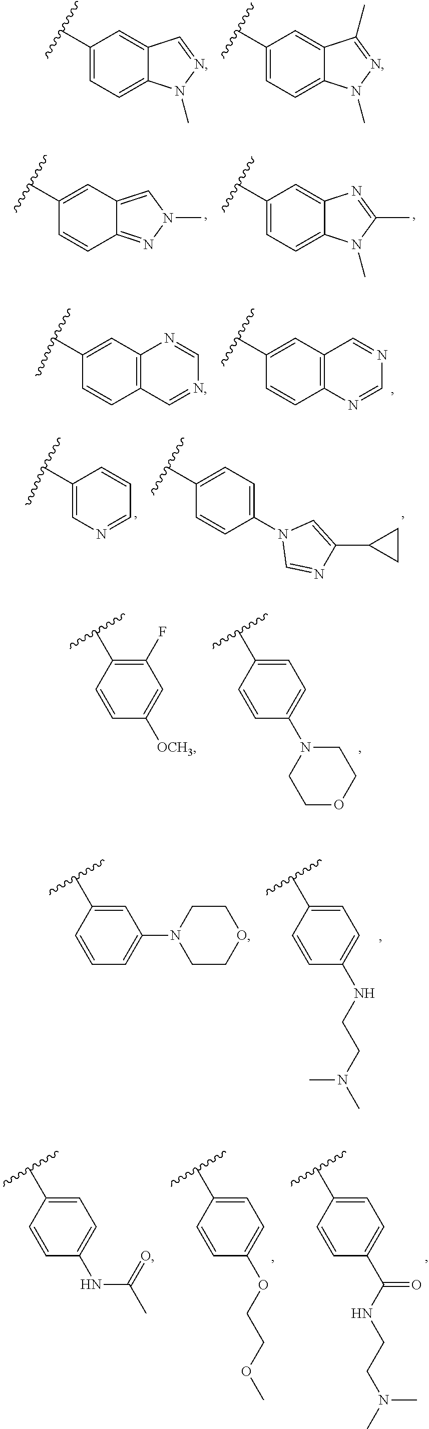

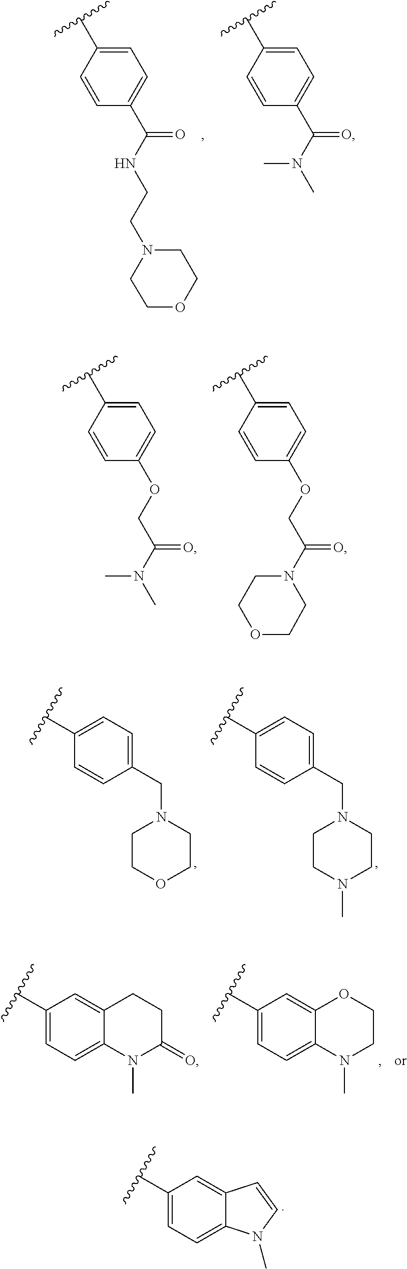

26. The compound of any one of claims 1-25, wherein X is N—R₄.

27. The compound of any one of claims 1-26, wherein R₄ is C₁₋₅alkyl or C₃₋₆cycloalkyl.

28. The compound of any one of claims 1-27, wherein R₄ is methyl, ethyl, isopropyl, cyclopropyl, —CH₂CF₃, or —CH₂CF₂.

29. The compound of any one of claims 1-28, wherein R₄ is methyl.

30. The compound of any one of claims 1-29, wherein Y is N.

31. The compound of any one of claims 1-30, wherein L is a bond.

32. The compound of any one of claims 1-31, wherein M is

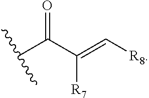

33. The compound of any one of claims 1-31, wherein M is

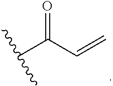 [SMILES: *C(=O)C=C]

34. The compound of any one of claims 1-33, wherein m is 0.

35. The compound of any one of claims 1-34, having the structure of Formula (I-1a1): 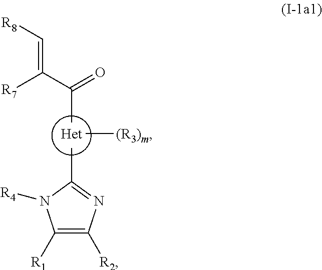
or a pharmaceutically acceptable salt, stereoisomer, or atropisomer thereof.

36. The compound of claim 35, wherein m is 0.

37. The compound of claim 35 or 36, wherein R₄ is C₁₋₅alkyl.

38. The compound of any one of claims 35-37, wherein R₄ is methyl.

39. The compound of any one of claims 1-32 and 34-38, wherein R₇ and R₈ are hydrogen.

40. The compound of any one of claims 1-39, having the structure of Formula (I-1 b2): 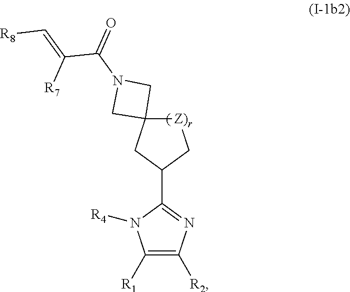
or a pharmaceutically acceptable salt, stereoisomer, or atropisomer thereof,
wherein:
Z is CH₂ or O; and
r is 0 or 1.

41. The compound of claim 40, wherein Z is CH₂.

42. The compound of claim 40 or 41, wherein r is 0.

43. The compound of any one of claims 40-42, wherein R₄ is C₁₋₅alkyl.

44. The compound of any one of claims 40-43, wherein R₄ is methyl.

45. The compound of any one of claims 40-44, wherein R₇ and R₈ are hydrogen.

46. The compound of any one of claims 1-45, having the structure of Formula (I-1c1): 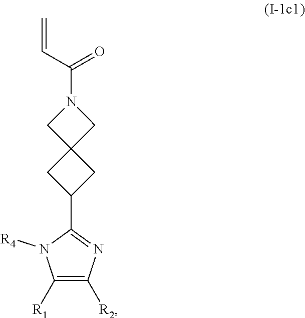
or a pharmaceutically acceptable salt, stereoisomer, or atropisomer thereof.

47. The compound of claim 46, wherein R₄ is C₁₋₅alkyl.

48. The compound of claim 46 or 47, wherein R₄ is methyl.

49. The compound of any one of claims 1-48, wherein R₁ is:

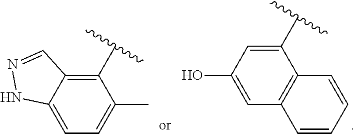 [SMILES: *c1c(C)ccc2[nH]ncc12 ; *c1cc(O)cc2ccccc12]

50. The compound of any one of claims 1-49, wherein R₂ is:

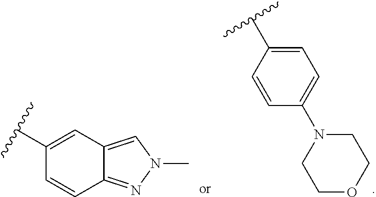 [SMILES: *c1ccc2nn(C)cc2c1 ; *c1ccc(N2CCOCC2)cc1]

51. The compound of any one of claims 1-50, wherein the compound is selected from the group consisting of:

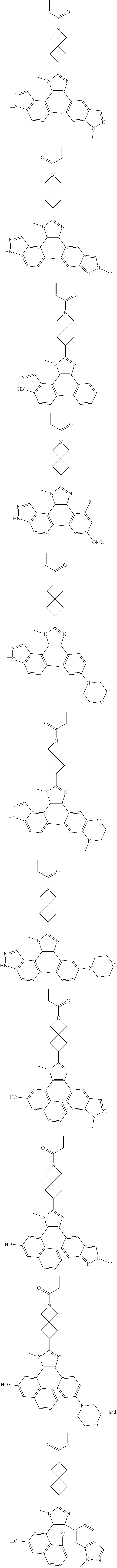

52. A composition comprising a compound of any one of claims 1-51 and a pharmaceutically acceptable excipient.

53. A method of treating a condition modulated by inhibition of KRAS proteins, comprising administering to a subject in need thereof a therapeutically effective amount of the compound of any one of claims 1-51 or a pharmaceutically acceptable salt thereof, or the composition of claim 52.

54. The method of claim 53, wherein the condition is associated with a KRAS mutation.

55. The method of claim 54, wherein the KRAS mutation is a G12C mutation.

56. The method of any one of claims 53-55, wherein the condition modulated by the inhibition of KRAS proteins is cancer.

57. A method of treating cancer, comprising administering to a subject in need thereof a therapeutically effective amount of the compound of any one of claims 1-51 or a pharmaceutically acceptable salt thereof, or the composition of claim 52.

58. The method of claim 56 or 57, wherein the cancer is selected from the group consisting of multiple myeloma, stomach cancer, bladder cancer, uterine cancer, esophageal squamous cell carcinoma, gastric cancer, glioblastomas, astrocytomas, retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, head and neck squamous cell carcinoma and cancer of the head and neck.

59. The compound of any one of claims 1-51 or the composition of claim 52 for use in the treatment of a condition modulated by inhibition of KRAS proteins.

60. The compound or composition for use of claim 59, wherein the condition is associated with a KRAS mutation.

61. The compound or composition for use of claim 60, wherein the KRAS mutation is a G12C mutation.

62. The compound or composition for use of any one of claims 59-61, wherein the condition modulated by the inhibition of KRAS proteins is cancer.

63. The compound of any one of claims 1-51 or the composition of claim 52 for use in the treatment of cancer.

64. The compound or composition for use of claim 62 or 63, wherein the cancer is selected from the group consisting of multiple myeloma, stomach cancer, bladder cancer, uterine cancer, esophageal squamous cell carcinoma, gastric cancer, glioblastomas, astrocytomas, retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, head and neck squamous cell carcinoma and cancer of the head and neck.

65. Use of the compound of any one of claims 1-51 or the composition of claim 52 in the manufacture of a medicament for the treatment of a condition modulated by inhibition of KRAS proteins.

66. The use of claim 65, wherein the condition is associated with a KRAS mutation.

67. The use of claim 66, wherein the KRAS mutation is a G12C mutation.

68. The use of any one of claims 65-67, wherein the condition modulated by the inhibition of KRAS proteins is cancer.

69. Use of the compound of any one of claims 1-51 or the composition of claim 52 in the manufacture of a medicament for the treatment of cancer.

70. The use of claim 68 or 69, wherein the cancer is selected from the group consisting of multiple myeloma, stomach cancer, bladder cancer, uterine cancer, esophageal squamous cell carcinoma, gastric cancer, glioblastomas, astrocytomas, retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, head and neck squamous cell carcinoma and cancer of the head and neck.
